(19) United States
(12) Patent Application Publication
(10) Pub. No.: US 2010/0041694 A1
(43) Pub. Date: Feb. 18, 2010

(54) AGRICULTURAL COMPOSITION FOR CONTROLLING OR PREVENTING PLANT DISEASES CAUSED BY PLANT PATHOGENIC MICROBES

(75) Inventors: Masanao Takaishi, Toyonaka-shi (JP); Takashi Komori, Nerima-ku (JP)

Correspondence Address: SUGHRUE MION, PLLC, 2100 PENNSYLVANIA AVENUE, N.W., SUITE 800, WASHINGTON, DC, 20037, US

(73) Assignee: Sumitomo Chemical Company, Limited, Chuo-ku, Tokyo (JP)

(21) Appl. No.: 12/532,096

(22) PCT Filed: Mar. 19, 2008
(86) PCT No.: PCT/JP2008/055808

§ 371 Date: Sep. 18, 2009

(30) Foreign Application Priority Data
Mar. 22, 2007 (JP) 2007-074372
Oct. 12, 2007 (JP) 2007-266279

(51) Int. Cl. A01N 43/40; C07D 215/00; A01N 43/42; C07D 277/62; A01N 43/78; C07D 471/02; A01P 1/00

(52) U.S. Cl. 514/300; 546/169; 514/311; 514/314; 548/152; 514/367; 546/122

(57) ABSTRACT

An agricultural composition comprising an amide compound represented by the following formula (I),

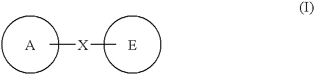

a salt thereof, or a hydrate thereof is effective for controlling or preventing plant diseases caused by plant pathogenic microbes when an effective amount of the agricultural composition is applied to useful crops.

### TECHNICAL FIELD

The present invention relates to an agricultural composition for controlling or preventing plant diseases caused by plant pathogenic microbes, comprising an amide compound, a salt thereof, or a hydrate thereof, and a method for controlling or preventing plant diseases caused by plant pathogenic microbes, which comprises applying an effective amount of the aforementioned agricultural composition to useful crops.

### BACKGROUND ART

A compound represented by formula (I) as shown below is known, and the production method thereof is also known (refer to Patent Document 1, for example). However, the ability of this compound to control or prevent plant pathogenic microbes except for Aspergillus is completely unknown.

A compound represented by the following formula (I), a salt thereof, or a hydrate thereof:

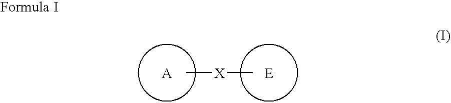

wherein A represents a 6-quinolyl group, a benzothiazol-6-yl group, or a [1.5]naphthylidin-2-yl group; X represents a group represented by formula —NH—C(═Y)—CH₂— or a group represented by formula —C(═Y)—NH—CH₂—; Y represents an oxygen atom, a sulfur atom, or NR^{Y} (wherein R^{Y} represents a C1-6 alkoxy group or a cyano group); and E represents a furyl group, a thienyl group, a pyrrolyl group, a tetrazolyl group, a thiazolyl group, a pyrazolyl group, or a phenyl group; wherein A may optionally have one to three substituents selected from the following substituent group a-1 and substituent group a-2, and E may optionally have one or two substituents selected from the following substituent group a-1 and substituent group a-2:
[Substituent Group a-1]
halogen atom, hydroxyl group, mercapto group, cyano group, carboxyl group, C1-6 alkyl group, C2-6 alkenyl group, C2-6 alkynyl group, C3-8 cycloalkyl group, C6-10 aryl group, 5- to 10-membered heterocyclic group, C3-8 cycloalkyl C1-6 alkyl group, C3-8 cycloalkylidene C1-6 alkyl group, C6-10 aryl C1-6 alkyl group, C6-10 aryl C2-6 alkenyl group, 5- to 10-membered heterocyclic C1-6 alkyl group, C1-6 alkoxy group, C2-6 alkenyloxy group, C2-6 alkynyloxy group, C3-8 cycloalkoxy group, C6-10 aryloxy group, C3-8 cycloalkyl C1-6 alkoxy group, C6-10 aryl C1-6 alkoxy group, 5- to 10-membered heterocyclic C1-6 alkoxy group, C1-6 alkylthio group, C2-6 alkenylthio group, C2-6 alkynylthio group, C3-8 cycloalkylthio group, C6-10 arylthio group, C3-8 cycloalkyl C1-6 alkylthio group, C6-10 aryl C1-6 alkylthio group, 5- to 10-membered heterocyclic C1-6 alkylthio group, mono-C1-6 alkylamino group, mono-C2-6 alkenylamino group, mono-C2-6 alkynylamino group, mono-C3-8 cycloalkylamino group, mono-C6-10 arylamino group, mono-C3-8 cycloalkyl C1-6 alkylamino group, mono-C6-10 aryl C1-6 alkylamino group, mono-5- to 10-membered heterocyclic C1-6 alkylamino group, di-C1-6 alkylamino group, N—C2-6 alkenyl-N—C1-6 alkylamino group, N—C2-6 alkynyl-N—C1-6 alkylamino group, N—C3-8 cycloalkyl-N—C1-6 alkylamino group, N—C6-10 aryl-N—C1-6 alkylamino group, N—C3-8 cycloalkyl C1-6 alkyl-N—C1-6 alkylamino group, N—C6-10 aryl C1-6 alkyl-N—C1-6 alkylamino group, N-5- to 10-membered heterocyclic C1-6 alkyl-N—C1-6 alkylamino group, C1-6 alkylcarbonyl group, C1-6 alkoxycarbonyl group, C1-6 alkylsulfonyl group, a group represented by formula —C(═N—Ra^{a1})R^{a2} (wherein R^{a1} represents a hydroxyl group or a C1-6 alkoxy group; and R^{a2} represents a hydrogen atom or a C1-6 alkyl group), C6-10 aryloxy C1-6 alkyl group, and 5- to 10-membered heterocyclic oxy C1-6 alkyl group; and
[Substituent Group a-2]
C1-6 alkyl group, C2-6 alkenyl group, C2-6 alkynyl group, C3-8 cycloalkyl group, C6-10 aryl group, 5- to 10-membered heterocyclic group, C3-8 cycloalkyl C1-6 alkyl group, C6-10 aryl C1-6 alkyl group, 5- to 10-membered heterocyclic C1-6 alkyl group, C1-6 alkoxy group, C2-6 alkenyloxy group, C2-6 alkynyloxy group, C3-8 cycloalkoxy group, C6-10 aryloxy group, C3-8 cycloalkyl C1-6 alkoxy group, C6-10 aryl C1-6 alkoxy group, 5- to 10-membered heterocyclic C1-6 alkoxy group, C1-6 alkylthio group, C2-6 alkenylthio group, C2-6 alkynylthio group, C3-8 cycloalkylthio group, C6-10 arylthio group, C3-8 cycloalkyl C1-6 alkylthio group, C6-10 aryl C1-6 alkylthio group, 5- to 10-membered heterocyclic C1-6 alkylthio group, mono-C1-6 alkylamino group, mono-C2-6 alkenylamino group, mono-C2-6 alkynylamino group, mono-C3-8 cycloalkylamino group, mono-C6-10 arylamino group, mono-C3-8 cycloalkyl C1-6 alkylamino group, mono-C6-10 aryl C1-6 alkylamino group, mono-5- to 10-membered heterocyclic C1-6 alkylamino group, di-C1-6 alkylamino group, N—C2-6 alkenyl-N—C1-6 alkylamino group, N—C2-6 alkynyl-N—C1-6 alkylamino group, N—C3-8 cycloalkyl-N—C1-6 alkylamino group, N—C6-10 aryl-N—C1-6 alkylamino group, N—C3-8 cycloalkyl C1-6 alkyl-N—C1-6 alkylamino group, N—C6-10 aryl C1-6 alkyl-N—C1-6 alkylamino group, N-5- to 10-membered heterocyclic C1-6 alkyl-N—C1-6 alkylamino group, C6-10 aryloxy C1-6 alkyl group, and 5- to 10-membered heterocyclic oxy C1-6 alkyl group;
wherein each group described in the substituent group a-2 has one to three substituents selected from the following substituent group b:

### [Substituent Group b]

halogen atom, hydroxyl group, mercapto group, cyano group, carboxyl group, amino group, carbamoyl group, nitro group, C1-6 alkyl group, C3-8 cycloalkyl group, C6-10 aryl group, 5- to 10-membered heterocyclic group, C1-6 alkoxy group, C6-10 aryloxy group, 5- to 10-membered heterocyclic oxy group, C1-6 alkoxycarbonyl group, C1-6 alkylsulfonyl group, trifluoromethyl group, trifluoromethoxy group, mono-C1-6 alkylamino group, di-C1-6 alkylamino group, mono-C6-10 arylamino group optionally having one amino group or one aminosulfonyl group, and N—C6-10 aryl C1-6 alkyl-N—C1-6 alkylamino group optionally having one amino group.
[Patent Document 1] International Patent Application WO2005/033079 pamphlet

### DISCLOSURE OF THE INVENTION

It is an object of the present invention to provide a novel use of the compound represented by formula (I) and the like.

As a result of intensive studies, the present inventors have found that the compound represented by formula (I) has an excellent effect of controlling or preventing plant diseases caused by plant pathogenic microbes, thereby completing the present invention.

That is to say, the present invention provides the following (1) to (3).

(1) An agricultural composition for controlling and preventing plant diseases caused by plant pathogenic microbes except for Aspergillus (hereinafter referred to as the agricultural composition of the present invention, at times), comprising a compound represented by formula (I), a salt thereof, or a hydrate thereof (hereinafter referred to as the present compound, at times):

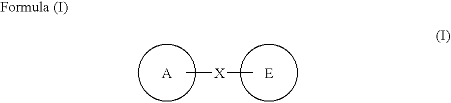

wherein A represents a 6-quinolyl group, a benzothiazol-6-yl group, or a [1.5]naphthylidin-2-yl group; X represents a group represented by formula —NH—C(═Y)—CH₂— or a group represented by formula —C(═Y)—NH—CH₂—;
Y represents an oxygen atom, a sulfur atom, or NR^{Y} (wherein R^{Y} represents a C1-6 alkoxy group or a cyano group); and E represents a furyl group, a thienyl group, a pyrrolyl group, a tetrazolyl group, a thiazolyl group, a pyrazolyl group, or a phenyl group;
wherein A may optionally have one to three substituents selected from the following substituent group a-1 and substituent group a-2, and E may optionally have one or two substituents selected from the following substituent group a-1 and substituent group a-2:
[Substituent Group a-1]
halogen atom, hydroxyl group, mercapto group, cyano group, carboxyl group, C1-6 alkyl group, C2-6 alkenyl group, C2-6 alkynyl group, C3-8 cycloalkyl group, C6-10 aryl group, 5- to 10-membered heterocyclic group, C3-8 cycloalkyl C1-6 alkyl group, C3-8 cycloalkylidene C1-6 alkyl group, C6-10 aryl C1-6 alkyl group, C6-10 aryl C2-6 alkenyl group, 5- to 10-membered heterocyclic C1-6 alkyl group, C1-6 alkoxy group, C2-6 alkenyloxy group, C2-6 alkynyloxy group, C3-8 cycloalkoxy group, C6-10 aryloxy group, C3-8 cycloalkyl C1-6 alkoxy group, C6-10 aryl C1-6 alkoxy group, 5- to 10-membered heterocyclic C1-6 alkoxy group, C1-6 alkylthio group, C2-6 alkenylthio group, C2-6 alkynylthio group, C3-8 cycloalkylthio group, C6-10 arylthio group, C3-8 cycloalkyl C1-6 alkylthio group, C6-10 aryl C1-6 alkylthio group, 5- to 10-membered heterocyclic C1-6 alkylthio group, mono-C1-6 alkylamino group, mono-C2-6 alkenylamino group, mono-C2-6 alkynylamino group, mono-C3-8 cycloalkylamino group, mono-C6-10 arylamino group, mono-C3-8 cycloalkyl C1-6 alkylamino group, mono-C6-10 aryl C1-6 alkylamino group, mono-5- to 10-membered heterocyclic C1-6 alkylamino group, di-C1-6 alkylamino group, N—C2-6 alkenyl-N—C1-6 alkylamino group, N—C2-6 alkynyl-N—C1-6 alkylamino group, N—C3-8 cycloalkyl-N—C1-6 alkylamino group, N—C6-10 aryl-N—C1-6 alkylamino group, N—C3-8 cycloalkyl C1-6 alkyl-N—C1-6 alkylamino group, N—C6-10 aryl C1-6 alkyl-N—C1-6 alkylamino group, N-5- to 10-membered heterocyclic C1-6 alkyl-N—C1-6 alkylamino group, C1-6 alkylcarbonyl group, C1-6 alkoxycarbonyl group, C1-6 alkylsulfonyl group, a group represented by formula —C(═N—R^{a1})R^{a2} (wherein R^{a1} represents a hydroxyl group or a C1-6 alkoxy group; and R^{a2} represents a hydrogen atom or a C1-6 alkyl group), C6-10 aryloxy C1-6 alkyl group, and 5- to 10-membered heterocyclic oxy C1-6 alkyl group; and
[Substituent Group a-2]
C1-6 alkyl group, C2-6 alkenyl group, C2-6 alkynyl group, C3-8 cycloalkyl group, C6-10 aryl group, 5- to 10-membered heterocyclic group, C3-8 cycloalkyl C1-6 alkyl group, C6-10 aryl C1-6 alkyl group, 5- to 10-membered heterocyclic C1-6 alkyl group, C1-6 alkoxy group, C2-6 alkenyloxy group, C2-6 alkynyloxy group, C3-8 cycloalkoxy group, C6-10 aryloxy group, C3-8 cycloalkyl C1-6 alkoxy group, C6-10 aryl C1-6 alkoxy group, 5- to 10-membered heterocyclic C1-6 alkoxy group, C1-6 alkylthio group, C2-6 alkenylthio group, C2-6 alkynylthio group, C3-8 cycloalkylthio group, C6-10 arylthio group, C3-8 cycloalkyl C1-6 alkylthio group, C6-10 aryl C1-6 alkylthio group, 5- to 10-membered heterocyclic C1-6 alkylthio group, mono-C1-6 alkylamino group, mono-C2-6 alkenylamino group, mono-C2-6 alkynylamino group, mono-C3-8 cycloalkylamino group, mono-C6-10 arylamino group, mono-C3-8 cycloalkyl C1-6 alkylamino group, mono-C6-10 aryl C1-6 alkylamino group, mono-5- to 10-membered heterocyclic C1-6 alkylamino group, di-C1-6 alkylamino group, N—C2-6 alkenyl-N—C1-6 alkylamino group, N—C2-6 alkynyl-N—C1-6 alkylamino group, N—C3-8 cycloalkyl-N—C1-6 alkylamino group, N—C6-10 aryl-N—C1-6 alkylamino group, N—C3-8 cycloalkyl C1-6 alkyl-N—C1-6 alkylamino group, N—C6-10 aryl C1-6 alkyl-N—C1-6 alkylamino group, N-5- to 10-membered heterocyclic C1-6 alkyl-N—C1-6 alkylamino group, C6-10 aryloxy C1-6 alkyl group, and 5- to 10-membered heterocyclic oxy C1-6 alkyl group;
wherein each group described in the substituent group a-2 has one to three substituents selected from the following substituent group b:

### [Substituent Group b]

halogen atom, hydroxyl group, mercapto group, cyano group, carboxyl group, amino group, carbamoyl group, nitro group, C1-6 alkyl group, C3-8 cycloalkyl group, C6-10 aryl group, 5- to 10-membered heterocyclic group, C1-6 alkoxy group, C6-10 aryloxy group, 5- to 10-membered heterocyclic oxy group, C1-6 alkoxycarbonyl group, C1-6 alkylsulfonyl group, trifluoromethyl group, trifluoromethoxy group, mono-C1-6 alkylamino group, di-C1-6 alkylamino group, mono-C6-10 arylamino group optionally having one amino group or one aminosulfonyl group, and N—C6-10 aryl C1-6 alkyl-N—C1-6 alkylamino group optionally having one amino group.
(2) A method for controlling or preventing plant diseases caused by plant pathogenic microbes except for Aspergillus, which comprises applying an effective amount of the agricultural composition of the present invention to useful crops.
(3) Use of the compound represented by formula (I), a salt thereof, or a hydrate thereof, of the present invention, in the manufacture of an agricultural composition.

According to the present invention, plant diseases caused by plant pathogenic microbes except for Aspergillus can be controlled or prevented.

### BEST MODE FOR CARRYING OUT THE INVENTION

A in formula (I) representing the present compound indicates a 6-quinolyl group optionally having one to three substituents selected from the following substituent group a-1 and substituent group a-2, a benzothiazol-6-yl group optionally having one to three substituents selected from the following substituent group a-1 and substituent group a-2, or a [1.5]naphthylidin-2-yl group optionally having one to three substituents selected from the following substituent group a-1 and substituent group a-2:

[Substituent Group a-1]
halogen atom, hydroxyl group, mercapto group, cyano group, carboxyl group, C1-6 alkyl group, C2-6 alkenyl group, C2-6 alkynyl group, C3-8 cycloalkyl group, C6-10 aryl group, 5- to 10-membered heterocyclic group, C3-8 cycloalkyl C1-6 alkyl group, C3-8 cycloalkylidene C1-6 alkyl group, C6-10 aryl C1-6 alkyl group, C6-10 aryl C2-6 alkenyl group, 5- to 10-membered heterocyclic C1-6 alkyl group, C1-6 alkoxy group, C2-6 alkenyloxy group, C2-6 alkynyloxy group, C3-8 cycloalkoxy group, C6-10 aryloxy group, C3-8 cycloalkyl C1-6 alkoxy group, C6-10 aryl C1-6 alkoxy group, 5- to 10-membered heterocyclic C1-6 alkoxy group, C1-6 alkylthio group, C2-6 alkenylthio group, C2-6 alkynylthio group, C3-8 cycloalkylthio group, C6-10 arylthio group, C3-8 cycloalkyl C1-6 alkylthio group, C6-10 aryl C1-6 alkylthio group, 5- to 10-membered heterocyclic C1-6 alkylthio group, mono-C1-6 alkylamino group, mono-C2-6 alkenylamino group, mono-C2-6 alkynylamino group, mono-C3-8 cycloalkylamino group, mono-C6-10 arylamino group, mono-C3-8 cycloalkyl C1-6 alkylamino group, mono-C6-10 aryl C1-6 alkylamino group, mono-5- to 10-membered heterocyclic C1-6 alkylamino group, di-C1-6 alkylamino group, N—C2-6 alkenyl-N—C1-6 alkylamino group, N—C2-6 alkynyl-N—C1-6 alkylamino group, N—C3-8 cycloalkyl-N—C1-6 alkylamino group, N—C6-10 aryl-N—C1-6 alkylamino group, N—C3-8 cycloalkyl C1-6 alkyl-N—C1-6 alkylamino group, N—C6-10 aryl C1-6 alkyl-N—C1-6 alkylamino group, N-5- to 10-membered heterocyclic C1-6 alkyl-N—C1-6 alkylamino group, C1-6 alkylcarbonyl group, C1-6 alkoxycarbonyl group, C1-6 alkylsulfonyl group, a group represented by formula —C(═N—R^{a1})R^{a2} (wherein R^{a1} represents a hydroxyl group or a C1-6 alkoxy group; and R^{a2} represents a hydrogen atom or a C1-6 alkyl group), C6-10 aryloxy C1-6 alkyl group, and 5- to 10-membered heterocyclic oxy C1-6 alkyl group; and
[Substituent Group a-2]
C1-6 alkyl group, C2-6 alkenyl group, C2-6 alkynyl group, C3-8 cycloalkyl group, C6-10 aryl group, 5- to 10-membered heterocyclic group, C3-8 cycloalkyl C1-6 alkyl group, C6-10 aryl C1-6 alkyl group, 5- to 10-membered heterocyclic C1-6 alkyl group, C1-6 alkoxy group, C2-6 alkenyloxy group, C2-6 alkynyloxy group, C3-8 cycloalkoxy group, C6-10 aryloxy group, C3-8 cycloalkyl C1-6 alkoxy group, C6-10 aryl C1-6 alkoxy group, 5- to 10-membered heterocyclic C1-6 alkoxy group, C1-6 alkylthio group, C2-6 alkenylthio group, C2-6 alkynylthio group, C3-8 cycloalkylthio group, C6-10 arylthio group, C3-8 cycloalkyl C1-6 alkylthio group, C6-10 aryl C1-6 alkylthio group, 5- to 10-membered heterocyclic C1-6 alkylthio group, mono-C1-6 alkylamino group, mono-C2-6 alkenylamino group, mono-C2-6 alkynylamino group, mono-C3-8 cycloalkylamino group, mono-C6-10 arylamino group, mono-C3-8 cycloalkyl C1-6 alkylamino group, mono-C6-10 aryl C1-6 alkylamino group, mono-5- to 10-membered heterocyclic C1-6 alkylamino group, di-C1-6 alkylamino group, N—C2-6 alkenyl-N—C1-6 alkylamino group, N—C2-6 alkynyl-N—C1-6 alkylamino group, N—C3-8 cycloalkyl-N—C1-6 alkylamino group, N—C6-10 aryl-N—C1-6 alkylamino group, N—C3-8 cycloalkyl C1-6 alkyl-N—C1-6 alkylamino group, N—C6-10 aryl C1-6 alkyl-N—C1-6 alkylamino group, N-5- to 10-membered heterocyclic C1-6 alkyl-N—C1-6 alkylamino group, C6-10 aryloxy C1-6 alkyl group, and 5- to 10-membered heterocyclic oxy C1-6 alkyl group;
wherein each group described in the substituent group a-2 has one to three substituents selected from the following substituent group b:

### [Substituent Group b]

halogen atom, hydroxyl group, mercapto group, cyano group, carboxyl group, amino group, carbamoyl group, nitro group, C1-6 alkyl group, C3-8 cycloalkyl group, C6-10 aryl group, 5- to 10-membered heterocyclic group, C1-6 alkoxy group, C6-10 aryloxy group, 5- to 10-membered heterocyclic oxy group, C1-6 alkoxycarbonyl group, C1-6 alkylsulfonyl group, trifluoromethyl group, trifluoromethoxy group, mono-C1-6 alkylamino group, di-C1-6 alkylamino group, mono-C6-10 arylamino group optionally having one amino group or one aminosulfonyl group, and N—C6-10 aryl C1-6 alkyl-N—C1-6 alkylamino group optionally having one amino group.

In addition, examples of such A may include a 6-quinolyl group optionally having one to three substituents selected from the following substituent group c-1 and substituent group c-2, a benzothiazol-6-yl group optionally having one to three substituents selected from the following substituent group c-1 and substituent group c-2, and a [1.5]naphthylidin-2-yl group optionally having one to three substituents selected from the following substituent group c-1 and substituent group c-2:

[Substituent Group c-1]
halogen atom, C1-6 alkyl group, C2-6 alkenyl group, C2-6 alkynyl group, C3-8 cycloalkyl group, C6-10 aryl group, 5- to 10-membered heterocyclic group, C3-8 cycloalkyl C1-6 alkyl group, C6-10 aryl C1-6 alkyl group, C6-10 aryl C2-6 alkenyl group, 5- to 10-membered heterocyclic C1-6 alkyl group, C1-6 alkoxy group, C2-6 alkenyloxy group, C2-6 alkynyloxy group, C3-8 cycloalkyl C1-6 alkoxy group, C6-10 aryl C1-6 alkoxy group, 5- to 10-membered heterocyclic C1-6 alkoxy group, mono-C2-6 alkylamino group, mono-C3-8 alkenylamino group, mono-C6-10 alkynylamino group, mono-C3-8 cycloalkylamino group, mono-C1-6 arylamino group, mono-C6-10 cycloalkyl C1-6 alkylamino group, mono-C1-6 aryl C1-6 alkylamino group, mono-5- to 10-membered heterocyclic C1-6 alkylamino group, C2-6 alkylcarbonyl group, and a group represented by formula —C(═N—OH)R^{a2} (wherein R^{a2} has the same meanings as defined above); and
[Substituent Group c-2]
C1-6 alkyl group, C2-6 alkenyl group, C2-6 alkynyl group, C3-8 cycloalkyl group, C6-10 aryl group, 5- to 10-membered heterocyclic group, C3-8 cycloalkyl C1-6 alkyl group, C6-10 aryl C1-6 alkyl group, 5- to 10-membered heterocyclic C1-6 alkyl group, C1-6 alkoxy group, C2-6 alkenyloxy group, C2-6 alkynyloxy group, C3-8 cycloalkyl C1-6 alkoxy group, C6-10 aryl C1-6 alkoxy group, 5- to 10-membered heterocyclic C1-6 alkoxy group, mono-C1-6 alkylamino group, mono-C2-6 alkenylamino group, mono-C2-6 alkynylamino group, mono-C3-8 cycloalkylamino group, mono-C6-10 arylamino group, mono-C3-8 cycloalkyl C1-6 alkylamino group, mono-C6-10 aryl C1-6 alkylamino group, and mono-5- to 10-membered heterocyclic C1-6 alkylamino group (wherein each group described in the substituent group c-2 has one to three substituents selected from the following substituent group d):

### [Substituent Group d]

halogen atom, hydroxyl group, carboxyl group, amino group, carbamoyl group, C1-6 alkoxy group, mono-C1-6 alkylamino group, di-C1-6 alkylamino group, mono-C6-10 arylamino group optionally having one amino group or one aminosulfonyl group, N—C6-10 aryl C1-6 alkyl-N—C1-6 alkylamino group optionally having one amino group, cyano group, C6-10 aryl group, 5- to 10-membered heterocyclic group, and C1-6 alkoxycarbonyl group.

Moreover, examples of such A may preferably include a 6-quinolyl group, a benzothiazol-6-yl group, and a [1.5]naphthylidin-2-yl group.

X in formula (I) representing the present compound indicates a group represented by formula —NH—C(═Y)—CH₂— and a group represented by formula —C(═Y)—NH—CH₂—. The aforementioned Y represents an oxygen atom, a sulfur atom, or NR^{Y} (wherein R^{Y} represents a C1-6 alkoxy group or a cyano group). X is preferably a group represented by the formula —NH—C(═Y)—CH₂—, and Y is preferably an oxygen atom.

E in formula (I) representing the present compound indicates a furyl group optionally having one or two substituents selected from the following substituent group a-1 and substituent group a-2, a thienyl group optionally having one or two substituents selected from the following substituent group a-1 and substituent group a-2, a pyrrolyl group optionally having one or two substituents selected from the following substituent group a-1 and substituent group a-2, or a phenyl group optionally having one or two substituents selected from the following substituent group a-1 and substituent group a-2:

[Substituent Group a-1]
halogen atom, hydroxyl group, mercapto group, cyano group, carboxyl group, C1-6 alkyl group, C2-6 alkenyl group, C2-6 alkynyl group, C3-8 cycloalkyl group, C6-10 aryl group, 5- to 10-membered heterocyclic group, C3-8 cycloalkyl C1-6 alkyl group, C3-8 cycloalkylidene C1-6 alkyl group, C6-10 aryl C1-6 alkyl group, C6-10 aryl C2-6 alkenyl group, 5- to 10-membered heterocyclic C1-6 alkyl group, C1-6 alkoxy group, C2-6 alkenyloxy group, C2-6 alkynyloxy group, C3-8 cycloalkoxy group, C6-10 aryloxy group, C3-8 cycloalkyl C1-6 alkoxy group, C6-10 aryl C1-6 alkoxy group, 5- to 10-membered heterocyclic C1-6 alkoxy group, C1-6 alkylthio group, C2-6 alkenylthio group, C2-6 alkynylthio group, C3-8 cycloalkylthio group, C6-10 arylthio group, C3-8 cycloalkyl C1-6 alkylthio group, C6-10 aryl C1-6 alkylthio group, 5- to 10-membered heterocyclic C1-6 alkylthio group, mono-C1-6 alkylamino group, mono-C2-6 alkenylamino group, mono-C2-6 alkynylamino group, mono-C3-8 cycloalkylamino group, mono-C6-10 arylamino group, mono-C3-8 cycloalkyl C1-6 alkylamino group, mono-C6-10 aryl C1-6 alkylamino group, mono-5- to 10-membered heterocyclic C1-6 alkylamino group, di-C1-6 alkylamino group, N—C2-6 alkenyl-N—C1-6 alkylamino group, N—C2-6 alkynyl-N—C1-6 alkylamino group, N—C3-8 cycloalkyl-N—C1-6 alkylamino group, N—C6-10 aryl-N—C1-6 alkylamino group, N—C3-8 cycloalkyl C1-6 alkyl-N—C1-6 alkylamino group, N—C6-10 aryl C1-6 alkyl-N—C1-6 alkylamino group, N-5- to 10-membered heterocyclic C1-6 alkyl-N—C1-6 alkylamino group, C1-6 alkylcarbonyl group, C1-6 alkoxycarbonyl group, C1-6 alkylsulfonyl group, a group represented by formula —C(═N—R^{a1})R^{a2} (wherein R^{a1} represents a hydroxyl group or a C1-6 alkoxy group; and R^{a2} represents a hydrogen atom or a C1-6 alkyl group), C6-10 aryloxy C1-6 alkyl group, and 5- to 10-membered heterocyclic oxy C1-6 alkyl group; and
[Substituent Group a-2]
C1-6 alkyl group, C2-6 alkenyl group, C2-6 alkynyl group, C3-8 cycloalkyl group, C6-10 aryl group, 5- to 10-membered heterocyclic group, C3-8 cycloalkyl C1-6 alkyl group, C6-10 aryl C1-6 alkyl group, 5- to 10-membered heterocyclic C1-6 alkyl group, C1-6 alkoxy group, C2-6 alkenyloxy group, C2-6 alkynyloxy group, C3-8 cycloalkoxy group, C6-10 aryloxy group, C3-8 cycloalkyl C1-6 alkoxy group, C6-10 aryl C1-6 alkoxy group, 5- to 10-membered heterocyclic C1-6 alkoxy group, C1-6 alkylthio group, C2-6 alkenylthio group, C2-6 alkynylthio group, C3-8 cycloalkylthio group, C6-10 arylthio group, C3-8 cycloalkyl C1-6 alkylthio group, C6-10 aryl C1-6 alkylthio group, 5- to 10-membered heterocyclic C1-6 alkylthio group, mono-C1-6 alkylamino group, mono-C2-6 alkenylamino group, mono-C2-6 alkynylamino group, mono-C3-8 cycloalkylamino group, mono-C6-10 arylamino group, mono-C3-8 cycloalkyl C1-6 alkylamino group, mono-C6-10 aryl C1-6 alkylamino group, mono-5- to 10-membered heterocyclic C1-6 alkylamino group, di-C1-6 alkylamino group, N—C2-6 alkenyl-N—C1-6 alkylamino group, N—C2-6 alkynyl-N—C1-6 alkylamino group, N—C3-8 cycloalkyl-N—C1-6 alkylamino group, N—C6-10 aryl-N—C1-6 alkylamino group, N—C3-8 cycloalkyl C1-6 alkyl-N—C1-6 alkylamino group, N—C6-10 aryl C1-6 alkyl-N—C1-6 alkylamino group, N-5- to 10-membered heterocyclic C1-6 alkyl-N—C1-6 alkylamino group, C6-10 aryloxy C1-6 alkyl group, and 5- to 10-membered heterocyclic oxy C1-6 alkyl group; wherein each group described in the substituent group a-2 has one to three substituents selected from the following substituent group b:

### [Substituent Group b]

halogen atom, hydroxyl group, mercapto group, cyano group, carboxyl group, amino group, carbamoyl group, nitro group, C1-6 alkyl group, C3-8 cycloalkyl group, C6-10 aryl group, 5- to 10-membered heterocyclic group, C1-6 alkoxy group, C6-10 aryloxy group, 5- to 10-membered heterocyclic oxy group, C1-6 alkoxycarbonyl group, C1-6 alkylsulfonyl group, trifluoromethyl group, trifluoromethoxy group, mono-C1-6 alkylamino group, di-C1-6 alkylamino group, mono-C6-10 arylamino group optionally having one amino group or one aminosulfonyl group, and N—C6-10 aryl C1-6 alkyl-N—C1-6 alkylamino group optionally having one amino group.

Preferably, such E may be a furyl group optionally having one or two substituents selected from the following substituent group e-1 and substituent group e-2, a thienyl group optionally having one or two substituents selected from the following substituent group e-1 and substituent group e-2, a pyrrolyl group optionally having one or two substituents selected from the following substituent group e-1 and substituent group e-2, or a phenyl group optionally having one or two substituents selected from the following substituent group e-1 and substituent group e-2:

[Substituent Group e-1]
halogen atom, C1-6 alkyl group, C2-6 alkenyl group, C2-6 alkynyl group, C6-10 aryl group, C3-8 cycloalkyl C1-6 alkyl group, C3-8 cycloalkylidene C1-6 alkyl group, C6-10 aryl C1-6 alkyl group, C6-10 aryl C2-6 alkenyl group, 5- to 10-membered heterocyclic C1-6 alkyl group, C1-6 alkoxy group, C2-6 alkenyloxy group, C2-6 alkynyloxy group, C6-10 aryloxy group, C3-8 cycloalkyl C1-6 alkoxy group, C6-10 aryl C1-6 alkoxy group, 5- to 10-membered heterocyclic C1-6 alkoxy group, C6-10 arylthio group, C6-10 aryl C1-6 alkylthio group, mono-C6-10 arylamino group, mono-C6-10 aryl C1-6 alkylamino group, N—C6-10 aryl-N—C1-6 alkylamino group, N—C6-10 aryl C1-6 alkyl-N—C1-6 alkylamino group, C6-10 aryloxy C1-6 alkyl group, and 5- to 10-membered heterocyclic oxy C1-6 alkyl group; and
[Substituent Group e-2]
C1-6 alkyl group, C2-6 alkenyl group, C2-6 alkynyl group, C6-10 aryl group, C3-8 cycloalkyl C1-6 alkyl group, C6-10 aryl C1-6 alkyl group, 5- to 10-membered heterocyclic C1-6 alkyl group, C1-6 alkoxy group, C2-6 alkenyloxy group, C2-6 alkynyloxy group, C6-10 aryloxy group, C3-8 cycloalkyl C1-6 alkoxy group, C6-10 aryl C1-6 alkoxy group, 5- to 10-membered heterocyclic C1-6 alkoxy group, C6-10 arylthio group, C6-10 aryl C1-6 alkylthio group, mono-C6-10 arylamino group, mono-C6-10 aryl C1-6 alkylamino group, N—C6-10 aryl-N—C1-6 alkylamino group, N—C6-10 aryl C1-6 alkyl-N—C1-6 alkylamino group, C6-10 aryloxy C1-6 alkyl group, and 5- to 10-membered heterocyclic oxy C1-6 alkyl group;
wherein each group described in the substituent group e-2 has one to three substituents selected from the following substituent group f:

### [Substituent Group f]

halogen atom, hydroxyl group, cyano group, amino group, nitro group, C3-8 cycloalkyl group, C1-6 alkoxy group, C6-10 aryloxy group, 5- to 10-membered heterocyclic oxy group, C1-6 alkoxycarbonyl group, C1-6 alkylsulfonyl group, mono-C6-10 arylamino group, trifluoromethyl group, trifluoromethoxy group, and C1-6 alkyl group.

More preferably, such E may be groups, wherein one or two hydrogen atoms of any one of a 2-furyl group, a 2-thienyl group, a pyrrolyl group, and a phenyl group may be substituted with [a fluorine atom, a chlorine atom, a C1-6 alkyl group (optionally substituted with one to three halogen atoms, C3-5 cycloalkyl groups, C1-6 alkoxy groups, phenoxy groups, 5- or 6-membered heterocyclic oxy groups, or C1-6 alkyl groups), a C2-6 alkenyl group (optionally substituted with one to three halogen atoms, C3-5 cycloalkyl groups, C1-6 alkoxy groups, phenoxy groups, 5- or 6-membered heterocyclic oxy groups, or C1-6 alkyl groups), a C2-6 alkynyl group (optionally substituted with one to three halogen atoms, C3-5 cycloalkyl groups, C1-6 alkoxy groups, phenoxy groups, 5- or 6-membered heterocyclic oxy groups, or C1-6 alkyl groups), a phenyl group (optionally substituted with one to three halogen atoms, C3-5 cycloalkyl groups, C1-6 alkoxy groups, or C1-6 alkyl groups), a C3-5 cycloalkyl C1-6 alkyl group (optionally substituted with one to three halogen atoms or C1-6 alkyl groups), a C3-5 cycloalkylidene C1-6 alkyl group (optionally substituted with one to three halogen atoms or C1-6 alkyl groups), a phenyl C1-6 alkyl group (optionally substituted with one to three halogen atoms, C3-5 cycloalkyl groups, C1-6 alkoxy groups, phenoxy groups, 5- or 6-membered heterocyclic oxy groups, or C1-6 alkyl groups), a phenyl C2-6 alkenyl group (optionally substituted with one to three halogen atoms, C3-5 cycloalkyl groups, C1-6 alkoxy groups, phenoxy groups, 5- or 6-membered heterocyclic oxy groups, or C1-6 alkyl groups), a 5- or 6-membered heterocyclic C1-6 alkyl group (optionally substituted with one to three halogen atoms, C3-5 cycloalkyl groups, C1-6 alkoxy groups, phenoxy groups, 5- or 6-membered heterocyclic oxy groups, or C1-6 alkyl groups), a C1-6 alkoxy group (optionally substituted with one to three halogen atoms or C1-6 alkyl groups), a C3-6 alkenyloxy group (optionally substituted with one to three halogen atoms or C1-6 alkyl groups), a C3-6 alkynyloxy group (optionally substituted with one to three halogen atoms or C1-6 alkyl groups), a phenoxy group (optionally substituted with one to three halogen atoms, C3-5 cycloalkyl groups, C1-6 alkoxy groups, phenoxy groups, 5- or 6-membered heterocyclic oxy groups, or C1-6 alkyl groups), a C3-6 cycloalkyl C1-6 alkoxy group optionally substituted with one to three halogen atoms, a phenyl C1-6 alkoxy group (optionally substituted with one to three halogen atoms, C3-5 cycloalkyl groups, C1-6 alkoxy groups, phenoxy groups, 5- or 6-membered heterocyclic oxy groups, or C1-6 alkyl groups), a 5- or 6-membered heterocyclic C1-6 alkoxy group (optionally substituted with one to three halogen atoms, C3-5 cycloalkyl groups, C1-6 alkoxy groups, phenoxy groups, 5- or 6-membered heterocyclic oxy groups, or C1-6 alkyl groups), a C6-10 aryloxy C1-6 alkyl group (optionally substituted with one to three halogen atoms, C3-5 cycloalkyl groups, C1-6 alkoxy groups, phenoxy groups, or 5- or 6-membered heterocyclic oxy groups), or a 5- or 6-membered heterocyclic oxy C1-6 alkyl group (optionally substituted with one to three halogen atoms, C3-5 cycloalkyl groups, or C1-6 alkoxy groups)].

Particularly preferably, such E may be groups, wherein one or two hydrogen atoms of any one of a 2-furyl group, a 2-thienyl group, and a phenyl group may be substituted with [a fluorine atom, a chlorine atom, a C1-6 alkyl group (optionally substituted with one to three halogen atoms, C3-5 cycloalkyl groups, C1-6 alkoxy groups, phenoxy groups, 5- or 6-membered heterocyclic oxy groups, or C1-6 alkyl groups), a C2-6 alkenyl group (optionally substituted with one to three halogen atoms, C3-5 cycloalkyl groups, C1-6 alkoxy groups, phenoxy groups, 5- or 6-membered heterocyclic oxy groups, or C1-6 alkyl groups), a C2-6 alkynyl group (optionally substituted with one to three halogen atoms, C3-5 cycloalkyl groups, C1-6 alkoxy groups, phenoxy groups, 5- or 6-membered heterocyclic oxy groups, or C1-6 alkyl groups), a phenyl group (optionally substituted with one to three halogen atoms, C3-5 cycloalkyl groups, C1-6 alkoxy groups, or C1-6 alkyl groups), a C3-5 cycloalkyl C1-6 alkyl group (optionally substituted with one to three halogen atoms or C1-6 alkyl groups), a C3-5 cycloalkylidene C1-6 alkyl group (optionally substituted with one to three halogen atoms or C1-6 alkyl groups), a phenyl C1-6 alkyl group (optionally substituted with one to three halogen atoms, C3-5 cycloalkyl groups, C1-6 alkoxy groups, phenoxy groups, 5- or 6-membered heterocyclic oxy groups, or C1-6 alkyl groups), a phenyl C2-6 alkenyl group (optionally substituted with one to three halogen atoms, C3-5 cycloalkyl groups, C1-6 alkoxy groups, phenoxy groups, 5- or 6-membered heterocyclic oxy groups, or C1-6 alkyl groups), a 5- or 6-membered heterocyclic C1-6 alkyl group (optionally substituted with one to three halogen atoms, C3-5 cycloalkyl groups, C1-6 alkoxy groups, phenoxy groups, 5- or 6-membered heterocyclic oxy groups, or C1-6 alkyl groups), a C1-6 alkoxy group (optionally substituted with one to three halogen atoms or C1-6 alkyl groups), a C3-6 alkenyloxy group (optionally substituted with one to three halogen atoms or C1-6 alkyl groups), a C3-6 alkynyloxy group (optionally substituted with one to three halogen atoms or C1-6 alkyl groups), a phenoxy group (optionally substituted with one to three halogen atoms, C3-5 cycloalkyl groups, C1-6 alkoxy groups, phenoxy groups, 5- or 6-membered heterocyclic oxy groups, or C1-6 alkyl groups), a C3-6 cycloalkyl C1-6 alkoxy group optionally substituted with one to three halogen atoms, a phenyl C1-6 alkoxy group (optionally substituted with one to three halogen atoms, C3-5 cycloalkyl groups, C1-6 alkoxy groups, phenoxy groups, 5- or 6-membered heterocyclic oxy groups, or C1-6 alkyl groups), a 5- or 6-membered heterocyclic C1-6 alkoxy group (optionally substituted with one to three halogen atoms, C3-5 cycloalkyl groups, C1-6 alkoxy groups, phenoxy groups, 5- or 6-membered heterocyclic oxy groups, or C1-6 alkyl groups), a C6-10 aryloxy C1-6 alkyl group (optionally substituted with one to three halogen atoms, C3-5 cycloalkyl groups, C1-6 alkoxy groups, phenoxy groups, or 5- or 6-membered heterocyclic oxy groups), and a 5- or 6-membered heterocyclic oxy C1-6 alkyl group (optionally substituted with one to three halogen atoms, C3-5 cycloalkyl groups, or C1-6 alkoxy groups)]; or a 3-pyrrolyl group whose N (nitrogen atom) is substituted with [a C1-6 alkyl group (optionally substituted with one to three halogen atoms, C3-5 cycloalkyl groups, C1-6 alkoxy groups, phenoxy groups, 5- or 6-membered heterocyclic oxy groups, or C1-6 alkyl groups), a C2-6 alkenyl group (optionally substituted with one to three halogen atoms, C3-5 cycloalkyl groups, C1-6 alkoxy groups, phenoxy groups, 5- or 6-membered heterocyclic oxy groups, or C1-6 alkyl groups), a C2-6 alkynyl group (optionally substituted with one to three halogen atoms, C3-5 cycloalkyl groups, C1-6 alkoxy groups, phenoxy groups, 5- or 6-membered heterocyclic oxy groups, or C1-6 alkyl groups), a phenyl group (optionally substituted with one to three halogen atoms, C3-5 cycloalkyl groups, C1-6 alkoxy groups, or C1-6 alkyl groups), a C3-5 cycloalkyl C1-6 alkyl group (optionally substituted with one to three halogen atoms or C1-6 alkyl groups), a C3-5 cycloalkylidene C1-6 alkyl group (optionally substituted with one to three halogen atoms or C1-6 alkyl groups), a phenyl C1-6 alkyl group (optionally substituted with one to three halogen atoms, C3-5 cycloalkyl groups, C1-6 alkoxy groups, phenoxy groups, 5- or 6-membered heterocyclic oxy groups, or C1-6 alkyl groups), a 5- or 6-membered heterocyclic C1-6 alkyl group (optionally substituted with one to three halogen atoms, C3-5 cycloalkyl groups, C1-6 alkoxy groups, phenoxy groups, 5- or 6-membered heterocyclic oxy groups, or C1-6 alkyl groups), a C1-6 alkoxy group (optionally substituted with one to three halogen atoms or C1-6 alkyl groups), a C3-6 alkenyloxy group (optionally substituted with one to three halogen atoms or C1-6 alkyl groups), a C3-6 alkynyloxy group (optionally substituted with one to three halogen atoms or C1-6 alkyl groups), a phenoxy group (optionally substituted with one to three halogen atoms, C3-5 cycloalkyl groups, C1-6 alkoxy groups, phenoxy groups, 5- or 6-membered heterocyclic oxy groups, or C1-6 alkyl groups), a C3-6 cycloalkyl C1-6 alkoxy group optionally substituted with one to three halogen atoms, a phenyl C1-6 alkoxy group (optionally substituted with one to three halogen atoms, C3-5 cycloalkyl groups, C1-6 alkoxy groups, phenoxy groups, 5- or 6-membered heterocyclic oxy groups, or C1-6 alkyl groups), a 5- or 6-membered heterocyclic C1-6 alkoxy group (optionally substituted with one to three halogen atoms, C3-5 cycloalkyl groups, C1-6 alkoxy groups, phenoxy groups, 5- or 6-membered heterocyclic oxy groups, or C1-6 alkyl groups), a C6-10 aryloxy C1-6 alkyl group (optionally substituted with one to three halogen atoms, C3-5 cycloalkyl groups, C1-6 alkoxy groups, phenoxy groups, or 5- or 6-membered heterocyclic oxy groups), or a 5- or 6-membered heterocyclic oxy C1-6 alkyl group (optionally substituted with one to three halogen atoms, C3-5 cycloalkyl groups, or C1-6 alkoxy groups)].

In the present specification, examples of a halogen atom may include a fluorine atom, a chlorine atom, and a bromine atom. Examples of a C1-6 alkyl group may include a methyl group, an ethyl group, an n-propyl group, an n-butyl group, an iso-butyl group, an n-pentyl group, an iso-pentyl group, an n-hexyl group, a 4-methylpentyl group, and a 3-methylpentyl group. Examples of a C2-6 alkenyl group may include a vinyl group, an allyl group, a 2-butenyl group, a 3-butenyl group, a 2-pentenyl group, a 3-pentenyl group, a prenyl group, a 3-methyl-3-butenyl group, a 2-hexenyl group, a 3-hexenyl group, a 4-hexenyl group, a 4-methyl-4-pentenyl group, and a 4-methyl-3-pentenyl group. Examples of a C2-6 alkynyl group may include an ethynyl group, a propargyl group, a 2-butynyl group, a 3-butynyl group, a 2-pentynyl group, a 3-pentynyl group, a 4-pentynyl group, a 2-hexynyl group, a 3-hexynyl group, a 4-hexynyl group, and a 5-hexynyl group. Examples of a C3-8 cycloalkyl group may include a cyclopropyl group, a cyclobutyl group, a cyclopentyl group, a cyclohexyl group, a cycloheptyl group, and a cyclooctyl group. Examples of a C6-10 aryl group may include a phenyl group, an indenyl group, and a naphthyl group. Examples of a 5- to 10-membered heterocyclic group may include a furyl group, a thienyl group, a pyridyl group, a benzofuranyl group, a benzothienyl group, a chromenyl group, an isocromenyl group, a thiocromenyl group, and an isothiocromenyl group. Examples of a C3-8 cycloalkyl C1-6 alkyl group may include a cyclopropylmethyl group, a cyclopropylethyl group, a cyclopropylpropyl group, a cyclopropylbutyl group, a cyclopropylpentyl group, a cyclopropylhexyl group, a cyclobutylmethyl group, a cyclobutylethyl group, a cyclobutylpropyl group, a cyclobutylbutyl group, a cyclobutylpentyl group, a cyclopentylethyl group, a cyclopentylpropyl group, a cyclopentylbutyl group, a cyclohexylethyl group, and a cyclohexylpropyl group. Examples of a C3-8 cycloalkylidene C1-6 alkyl group may include a cyclopropylidenemethyl group, a cyclopropylideneethyl group, a cyclopropylidenepropyl group, a cyclopropylidenebutyl group, a cyclopropylidenepentyl group, a cyclopropylidenehexyl group, a cyclobutylidenemethyl group, a cyclobutylideneethyl group, a cyclobutylidenepropyl group, a cyclobutylidenebutyl group, a cyclobutylidenepentyl group, a cyclopentylideneethyl group, a cyclopentylidenepropyl group, a cyclopentylidenebutyl group, a cyclohexylideneethyl group, and a cyclohexylidenepropyl group. Examples of a C6-10 aryl C1-6 alkyl group may include a benzyl group, a phenethyl group, a phenylpropyl group, a phenylbutyl group, a phenylpentyl group, a 2-methyl-4-phenylbutyl group, a 2-methyl-5-phenylpentyl group, a 3-methyl-5-phenylpentyl group, and a (2-naphthyl)ethyl group. Examples of a 5- to 10-membered heterocyclic C1-6 alkyl group may include a furylmethyl group, a furylethyl group, a furylpropyl group, a furylbutyl group, a furylpentyl group, a furylhexyl group, a thienylethyl group, a thienylmethyl group, a thienylpropyl group, a thienylbutyl group, a thienylpentyl group, and a thienylhexyl group. Examples of a C1-6 alkoxy group may include a methoxy group, an ethoxy group, an n-propyloxy group, an iso-propoxy group, an n-butoxy group, an iso-butoxy group, a sec-butoxy group, an n-pentyloxy group, an iso-pentyloxy group, a sec-pentyloxy group, an n-hexyloxy group, an iso-hexyloxy group, a sec-hexyloxy group, and a 2,3-dimethylbutoxy group. Examples of a C2-6 alkenyloxy group may include a vinyloxy group, an allyloxy group, a 1-methyl-2-propenyloxy group a 2-butenyloxy group, a 3-butenyloxy group, a 2-pentenyloxy group, a 3-pentenyloxy group, a prenyloxy group, a 3-methyl-3-butenyloxy group, a 2-hexenyloxy group, a 3-hexenyloxy group, a 4-hexenyloxy group, a 4-methyl-4-pentenyloxy group, and a 4-methyl-3-pentenyloxy group. Examples of a C2-6 alkynyloxy group may include a propargyloxy group, a 2-butynyloxy group, a 3-butynyloxy group, a 2-pentynyloxy group, a 3-pentynyloxy group, a 4-pentynyloxy group, a 2-hexynyloxy group, a 3-hexynyloxy group, a 4-hexynyloxy group, and a 5-hexynyloxy group. Examples of a C3-8 cycloalkoxy group may include a cyclopropyloxy group, a cyclobutyloxy group, a cyclopentyloxy group, a cyclohexyloxy group, a cycloheptyloxy group, and a cyclooctyloxy group. Examples of a C6-10 aryloxy group may include a phenoxy group and a naphthoxy group. Examples of a C3-8 cycloalkyl C1-6 alkoxy group may include a cyclopropylmethoxy group, a cyclopropylethoxy group, a cyclopropylpropoxy group, a cyclopropylbutoxy group, a cyclopropylpentyloxy group, a cyclopropylhexyloxy group, a cyclobutylmethoxy group, a cyclobutylethoxy group, a cyclobutylpropoxy group, a cyclobutylbutoxy group, a cyclobutylpentyloxy group, a cyclopentylethoxy group, a cyclopentylpropoxy group, a cyclopentylbutoxy group, a cyclohexylethoxy group, and a cyclohexylpropoxy group. Examples of a C6-10 aryl C1-6 alkoxy group may include a benzyloxy group, a phenethyloxy group, a phenylpropyloxy group, a phenylbutyloxy group, a phenylpentyloxy group, a 2-methyl-4-phenylbutyloxy group, a 2-methyl-5-phenylpentyloxy group, a 3-methyl-5-phenylpentyloxy group and a (2-naphthyl)ethyloxy group. Examples of a 5- to 10-membered heterocyclic C1-6 alkoxy group may include a furylmethoxy group, a furylethoxy group, a furylpropyloxy group, a furylbutyloxy group, a furylpentyloxy group, a furylhexyloxy group, a thienylmethoxy group, a thienylethoxy group, a thienylpropyloxy group, a thienylbutyloxy group, a thienylpentyloxy group, and a thienylhexyloxy group. Examples of a C1-6 alkylthio group may include a methylthio group, an ethylthio group, an n-propylthio group, an n-butylthio group, an iso-butylthio group, an n-penylthio group, an iso-pentylthio group, an n-hexylthio group, a 4-methylpentylthio group, and a 3-methylpentylthio group. Examples of a C2-6 alkenylthio group may include a vinylthio group, an allylthio group, a 1-methyl-2-propenylthio group, a 2-butenylthio group, a 3-butenylthio group, a 2-pentenylthio group, a 3-pentenylthio group, a prenylthio group, a 3-methyl-3-butenylthio group, a 2-hexenylthio group, a 3-hexenylthio group, a 4-hexenylthio group, a 4-methyl-4-pentenylthio group, and a 4-methyl-3-pentenylthio group. Examples of a C2-6 alkynylthio group may include a propargylthio group, a 2-butynylthio group, a 3-butynylthio group, a 2-pentynylthio group, a 3-pentynylthio group, a 4-pentynylthio group, a 2-hexynylthio group, a 3-hexynylthio group, a 4-hexynylthio group, and a 5-hexynylthio group. Examples of a C3-8 cycloalkylthio group may include a cyclopropylthio group, a cyclobutylthio group, a cyclopentylthio group, a cyclohexylthio group, a cycloheptylthio group, and a cyclooctylthio group. Examples of a C6-10 arylthio group may include phenylthio and naphthylthio. Examples of a C3-8 cycloalkyl C1-6 alkylthio group may include a cyclopropylmethylthio group, a cyclopropylethylthio group, a cyclopropylpropylthio group, a cyclopropylbutylthio group, a cyclopropylpentylthio group, a cyclopropylhexylthio group, a cyclobutylmethylthio group, a cyclobutylethylthio group, a cyclobutylpropylthio group, a cyclobutylbutylthio group, a cyclobutylpentylthio group, a cyclopentylethylthio group, a cyclopentylpropylthio group, a cyclopentylbutylthio group, a cyclohexylethylthio group, and a cyclohexylpropylthio group. Examples of a C6-10 aryl C1-6 alkylthio group may include a benzylthio group, a phenethylthio group, a phenylpropylthio group, a phenylbutylthio group, a phenylpentylthio group, a 2-methyl-4-phenylbutylthio group, a 2-methyl-5-phenylpentylthio group, and a 3-methyl-5-phenylpentylthio group. Examples of a 5- to 10-membered heterocyclic C1-6 alkylthio group may include a furylmethylthio group, a furylethylthio group, a furylpropylthio group, a furylbutylthio group, a furylpentylthio group, a furylhexylthio group, a thienylethylthio group, a thienylmethylthio group, a thienylpropylthio group, a thienylbutylthio group, a thienylpentylthio group, and a thienylhexylthio group. Examples of a mono-C1-6 alkylamino group may include a methylamino group, an ethylamino group, an n-propylamino group, an n-butylamino group, an iso-butylamino group, an n-pentylamino group, an iso-pentylamino group, an n-hexylamino group, a 4-methylpentylamino group, and a 3-methylpentylamino group. Examples of a mono-C2-6 alkenylamino group may include an allylamino group, a 2-butenylamino group, a 3-butenylamino group, a 2-pentenylamino group, a 3-pentenylamino group, a prenylamino group, a 3-methyl-3-butenylamino group, a 2-hexenylamino group, a 3-hexenylamino group, a 4-hexenylamino group, a 4-methyl-4-pentenylamino group, and a 4-methyl-3-pentenylamino group. Examples of a mono-C2-6 alkynylamino group may include a propargylamino group, a 2-butynylamino group, a 3-butynyl group amino, a 2-pentynylamino group, a 3-pentynylamino group, a 4-pentynylamino group, a 2-hexynylamino group, a 3-hexynylamino group, a 4-hexynylamino group, and a 5-hexynylamino group. Examples of a mono-C3-8 cycloalkylamino group may include a cyclopropylamino group, a cyclobutylamino group, a cyclopentylamino group, a cyclohexylamino group, a cycloheptylamino group, and a cyclooctylamino group. Examples of a mono-C6-10 arylamino group may include an alanyl group and a naphthylamino group. Examples of a mono-C3-8 cycloalkyl C1-6 alkylamino group may include a cyclopropylmethylamino group, a cyclopropylethylamino group, a cyclopropylpropylamino group, a cyclopropylbutylamino group, a cyclopropylpentylamino group, a cyclopropylhexylamino group, a cyclobutylmethylamino group, a cyclobutylethylamino group, a cyclobutylpropylamino group, a cyclobutylbutylamino group, a cyclobutylpentylamino group, a cyclopentylethylamino group, a cyclopentylpropylamino group, a cyclopentylbutylamino group, a cyclohexylethylamino group, and a cyclohexylpropylamino group. Examples of a mono-C6-10 aryl C1-6 alkylamino group may include a benzylamino group, a phenethylamino group, a phenylpropylamino group, a phenylbutylamino group, a phenylpentylamino group, a 2-methyl-4-phenylbutylamino group, a 2-methyl-5-phenylpentylamino group, a 3-methyl-5-phenylpentylamino group, and a (2-naphthyl)ethylamino group. Examples of a mono-5- to 10-membered heterocyclic C1-6 alkylamino group may include a furylmethylamino group, a furylethylamino group, a furylpropylamino group, a furylbutylamino group, a furylpentylamino group, a furylhexylamide group, a thienylethylamino group, a thienylmethylamino group, a thienylpropylamino group, a thienylbutylamino group, a thienylpentylamino group, and a thienylhexylamino group. Examples of a di-C1-6 alkylamino group may include a dimethylamino group, a methylethylamino group, a diethylamino group, a methylpropylamino group, and a methylbutylamino group. Examples of an N—C2-6 alkenyl-N—C1-6 alkylamino group may include an N-allyl-N-methylamino group, an N-(2-butynyl)-N-methylamino group, and an N-(3-butenyl)-N-methylamino group. Examples of an N—C2-6 alkynyl-N—C1-6 alkylamino group may include an N-methyl-N-propargylamino group, an N-(2-butenyl)-N-methylamino group, and an N-(3-butynyl)-N-methylamino group. Examples of an N—C3-8 cycloalkyl-N—C1-6 alkylamino group may include an N-cyclopropyl-N-methylamino group, an N-cyclobutyl-N-methylamino group, and an N-cyclopentyl-N-methylamino group. An example of an N—C6-10 aryl-N—C1-6 alkylamino group is an N-methylanilino group. Examples of an N—C3-8 cycloalkyl C1-6 alkyl-N—C1-6 alkylamino group may include an N-cyclopropylmethyl-N-methylamino group, an N-cyclopropylethyl-N-methylamino group, an N-cyclopropylpropyl-N-methylamino group, an N-cyclobutylmethyl-N-methylamino group, an N-cyclobutylethyl-N-methylamino group, and an N-cyclobutylpropyl-N-methylamino group. Examples of an N—C6-10 aryl C1-6 alkyl-N—C1-6 alkylamino group may include an N-benzyl-N-methylamino group, an N-phenethyl-N-methylamino group, an N-phenylpropyl-N-methylamino group, an N-phenylbutyl-N-methylamino group, and an N-phenylpentyl-N-methylamino group. Examples of an N-5- to 10-membered heterocyclic C1-6 alkyl-N—C1-6 alkylamino group, C1-6 alkylcarbonyl group may include an N-furylmethyl-N-methylamino group, an N-furylethyl-N-methylamino group, an N-furylpropyl-N-methylamino group, an N-furylbutyl-N-methylamino group, an N-furylpentyl-N-methylamino group, an N-thienyl-N-methylamino group, an N-thienylethyl-N-methylamino group, an N-thienylpropyl-N-methylamino group, an N-thienylbutyl-N-methylamino group, and an N-thienylpentyl-N-methylamino group. Examples of a C1-6 alkoxycarbonyl group may include a methoxycarbonyl group, an ethoxycarbonyl group, an n-propyloxycarbonyl group, an iso-propoxycarbonyl group, an n-butoxycarbonyl group, an iso-butoxycarbonyl group, a sec-butoxycarbonyl group, an n-pentyloxycarbonyl group, an iso-pentyloxycarbonyl group, a sec-pentyloxycarbonyl group, an n-hexyloxycarbonyl group, an iso-hexyloxycarbonyl group, and a sec-hexyloxycarbonyl group. Examples of a C1-6 alkylsulfonyl group may include a methanesulfonyl group, an ethanesulfonyl group, a propanesulfonyl group, a butanesulfonyl group, a pentanesulfonyl group, and a hexanesulfonyl group. Examples of a group represented by formula —C(═N—R^{a1})R^{a2} may include a hydroxyiminomethyl group, a hydroxyiminoethyl group, a methoxyiminomethyl group, an ethoxyiminomethyl group, and a methoxyiminoethyl group. Examples of a C6-10 aryloxy C1-6 alkyl group may include a benzyloxymethyl group, a benzyloxyethyl group, a benzyloxypropyl group, a benzyloxybutyl group, a benzyloxypentyl group, and a (2-naphthyl)oxymethyl group. Examples of a 5- to 10-membered heterocyclic oxy C1-6 alkyl group may include a furyloxymethyl group, a furyloxyethyl group, a furyloxypropyl group, a furyloxybutyl group, a furyloxypentyl group, a thienyloxymethyl group, a thienyloxyethyl group, a thienyloxypropyl group, a thienyloxybutyl group, a thienyloxypentyl group, a pyridyloxymethyl group, a pyridyloxyethyl group, a benzofuranyloxymethyl group, and a benzothienyloxymethyl group. Examples of a C6-10 aryl C2-6 alkenyl group may include a styryl group and a phenylpropenyl group.

More specific examples of such E may include a phenyl group, a 2-furyl group, a 3-furyl group, a 2-thienyl group, a 3-thienyl group, a 5-phenylfuran-2-yl group, a 5-phenoxyfuran-2-yl group, a 5-(4-fluorophenoxy)furan-2-yl group, a 5-(3-fluorophenoxy)furan-2-yl group, a 5-(4-methylphenoxy)furan-2-yl group, a 5-(4-chlorophenoxy)furan-2-yl group, a 5-(3-chlorophenoxy)furan-2-yl group, a 5-(3-methylphenoxy)furan-2-yl group, a 5-(4-methoxyphenoxy)furan-2-yl group, a 5-(3-methoxyphenoxy)furan-2-yl group, a 5-benzylfuran-2-yl group, a 5-(4-fluorophenyl)methylfuran-2-yl group, a 5-(3-fluorophenyl)methylfuran-2-yl group, a 5-(4-methylphenyl)methylfuran-2-yl group, a 5-(4-chlorophenyl)methylfuran-2-yl group, a 5-(3-chlorophenyl)methylfuran-2-yl group, a 5-(3-methylphenyl)methylfuran-2-yl group, a 5-(4-methoxyphenyl)methylfuran-2-yl group, a 5-(3-methoxyphenyl)methylfuran-2-yl group, a 5-phenoxythiophen-2-yl group, a 5-(4-fluorophenoxy)thiophen-2-yl group, a 5-(3-fluorophenoxy)thiophen-2-yl group, a 5-(2-fluorophenoxy)thiophen-2-yl group, a 5-(4-methylphenoxy)thiophen-2-yl group, a 5-(4-chlorophenoxy)thiophen-2-yl group, a 5-(3-chlorophenoxy)thiophen-2-yl group, a 5-(3-methylphenoxy)thiophen-2-yl group, a 5-(4-methoxyphenoxy)thiophen-2-yl group, a 5-(3-methoxyphenoxy)thiophen-2-yl group, a 5-(3-cyanophenoxy)thiophen-2-yl group, a 5-benzyloxythiophen-2-yl group, a 5-benzylthiophen-2-yl group, a 5-(4-fluorophenyl)methylthiophen-2-yl group, a 5-(3-fluorophenyl)methylthiophen-2-yl group, a 5-(4-methylphenyl)methylthiophen-2-yl group, a 5-(4-chlorophenyl)methylthiophen-2-yl group, a 5-(3-chlorophenyl)methylthiophen-2-yl group, a 5-(3-methylphenyl)methylthiophen-2-yl group, a 5-(4-methoxyphenyl)methylthiophen-2-yl group, a 5-(3-methoxyphenyl)methylthiophen-2-yl group, a 5-(2-thienyl)methylthiophen-2-yl group, a 5-(2-pyridyl)methylthiophen-2-yl group, a 5-(2-benzofuranyl)methylthiophen-2-yl group, a 5-phenoxythiophen-3-yl group, a 5-(4-fluorophenoxy)thiophen-3-yl group, a 5-(3-fluorophenoxy)thiophen-3-yl group, a 5-(4-methylphenoxy)thiophen-3-yl group, a 5-(4-chlorophenoxy)thiophen-3-yl group, a 5-(3-chlorophenoxy)thiophen-3-yl group, a 5-(3-methylphenoxy)thiophen-3-yl group, a 5-(4-methoxyphenoxy)thiophen-3-yl group, a 5-(3-methoxyphenoxy)thiophen-3-yl group, a 3-isobutylphenyl group, a 3-(2-isobutenyl)phenyl group, a 3-isoprenyloxyphenyl group, a 3-cyclopentylmethylidenephenyl group, a 3-biphenyl group, 3-benzylphenyl group, a 3-phenylaminophenyl group, a 3-phenylthiophenyl group, a 3-phenoxyphenyl group, a 3-(3-fluorophenoxy)phenyl group, a 3-(4-fluorophenoxy)phenyl group, a 3-(3-cyanophenoxy)phenyl group, a 3-(3-methoxyphenoxy)phenyl group, a 3-(3-fluorophenoxy)phenyl group, a 3-benzylaminophenyl group, a 3-(N-benzyl-N-methylamino)phenyl group, a 3-benzyloxyphenyl group, a 3-(3-methylbutoxy)phenyl group, a 3-cyclopropylmethoxyphenyl group, a 4-benzylphenyl group, a 4-phenoxyphenyl group, a 4-phenylthiophenyl group, a 4-benzyloxyphenyl group, a 4-(3-fluorobenzyloxy)phenyl group, a 4-(3-chlorobenzyloxy)phenyl group, a 4-(3-methylbenzyloxy)phenyl group, a 4-(3-methoxybenzyloxy)phenyl group, a 4-(2-pyridylmethyloxy)phenyl group, a 4-(2-furylmethoxy)phenyl group, a 4-(3-furylmethoxy)phenyl group, a 4-(2-thienylmethoxy)phenyl group, a 4-(3-thienylmethoxy)phenyl group, a 4-(5-benzo[1,3]dioxolylmethoxy)phenyl group, a 4-(6-fluoropyridin-2-yloxy)methylphenyl group, a 4-phenoxymethylphenyl group, a 4-phenethylphenyl group, a 4-benzylaminophenyl group, a 4-(N-benzyl-N-methylamino)phenyl group, a 4-phenylaminomethylphenyl group, a (Z)-4-styrylphenyl group, a 1-benzylpyrrol-3-yl group, a 1-phenethylpyrrol-3-yl group, and a 1-(5-benzo[1,3]dioxolyl)pyrrol-3-yl group.

A preferred example of the present compound is a compound wherein, in formula (I), X represents a group represented by formula [—C(═O)—NH—CH₂—], A represents a 6-quinolyl group, a [1.5]naphthylidin-2-yl group, or a benzothiazol-6-yl group, and E represents a 2-furyl group, a 2-thienyl group, a 3-pyrrolyl group, or a phenyl group (wherein E may optionally have one or two substituents selected from the substituent group g-1 or substituent group g-2).

More specific examples of the present compound are as shown in Table-1.

| No. | A | X | E |
|---|---|---|---|
| 1 | 6-quinolyl | —C(═O)NHCH2- | phenyl |
| 2 | 6-quinolyl | —C(═O)NHCH2- | 4-benzyloxyphenyl |
| 3 | 6-quinolyl | —C(═O)NHCH2- | 4-phenoxyphenyl |
| 4 | 6-quinolyl | —C(═O)NHCH2- | 3-biphenyl |
| 5 | 6-quinolyl | —C(═O)NHCH2- | 3-phenoxyphenyl |
| 6 | 6-quinolyl | —C(═O)NHCH2- | 3-benzyloxyphenyl |
| 7 | 6-quinolyl | —C(═O)NHCH2- | 3-hydroxyphenyl |
| 8 | 6-quinolyl | —C(═O)NHCH2- | 4-hydroxyphenyl |
| 9 | 6-quinolyl | —C(═O)NHCH2- | 4-benzyloxy-2-fluorophenyl |
| 10 | 6-quinolyl | —C(═O)NHCH2- | 4-(3-fluorobenzyloxy)phenyl |
| 11 | 6-quinolyl | —C(═O)NHCH2- | 3-(4-fluorophenyl)oxyphenyl |
| 12 | 6-quinolyl | —C(═O)NHCH2- | 3-(3-fluorophenyl)oxyphenyl |
| 13 | 6-quinolyl | —C(═O)NHCH2- | 4-(1,3-dioxolidin-2-yl)phenyl |
| 14 | 6-quinolyl | —C(═O)NHCH2- | 4-(2-phenyl)ethyl)phenyl |
| 15 | 6-benzothiazolyl | —NHC(═O)CH2- | phenyl |
| 16 | 6-quinolyl | —C(═O)NHCH2- | 3-cyclopropylmethoxyphenyl |
| 17 | 6-quinolyl | —C(═O)NHCH2- | 4-(2-pyridyl)methoxyphenyl |
| 18 | 6-quinolyl | —C(═O)NHCH2- | 3-(3-methylbutyloxy)phenyl |
| 19 | 6-quinolyl | —C(═O)NHCH2- | 3-anilinophenyl |
| 20 | 6-quinolyl | —C(═O)NHCH2- | 3-Bromophenyl |
| 21 | 6-quinolyl | —C(═O)NHCH2- | 4-benzylphenyl |
| 22 | 6-quinolyl | —C(═O)NHCH2- | 3-benzylphenyl |
| 23 | 6-quinolyl | —C(═O)NHCH2- | 3-(2-methyl-1-propenyl)phenyl |
| 24 | 6-quinolyl | —C(═O)NHCH2- | 3-(2-methylpropyl)phenyl |
| 25 | 6-quinolyl | —NHC(═O)CH2- | 4-phenoxyphenyl |
| 26 | 6-quinolyl | —NHC(═O)CH2- | 3-methoxyphenyl |
| 27 | 6-quinolyl | —C(═O)NHCH2- | 2-phenoxythiazol-5-yl |
| 28 | 6-quinolyl | —NHC(═O)CH2- | 3-phenoxyphenyl |
| 29 | 6-quinolyl | —C(═O)NHCH2- | 2-(5-phenoxy)thienyl |
| 30 | 6-quinolyl | —C(═O)NHCH2- | 2-(5-phenoxy)furyl |
| 31 | 6-benzothiazolyl | —C(═O)NHCH2- | 4-benzyloxyphenyl |
| 32 | 6-benzothiazolyl | —C(═O)NHCH2- | 3-phenoxyphenyl |
| 33 | 6-benzothiazolyl | —C(═O)NHCH2- | phenyl |
| 34 | 6-benzothiazolyl | —C(═O)NHCH2- | 4-(3-fluorobenzyloxy)phenyl |
| 35 | 6-benzothiazolyl | —C(═O)NHCH2- | 2-[5-(3-fluorophenoxy)]thienyl |
| 36 | 6-benzothiazolyl | —C(═O)NHCH2- | 2-(5-phenoxy)thienyl |
| 37 | 6-benzothiazolyl | —NHC(═O)CH2- | 3-phenoxyphenyl |
| 38 | [1.5]-naphtyridin-2-yl | —C(═O)NHCH2- | 3-phenoxyphenyl |
| 39 | [1.5]-naphtyridin-2-yl | —C(═O)NHCH2- | 4-benzyloxyphenyl |
| 40 | 6-quinolyl | —C(═O)NHCH2- | 4-methoxy-3-methylphenyl |
| 41 | 6-quinolyl | —C(═O)NHCH2- | 3-chlorophenyl |
| 42 | 6-quinolyl | —C(═O)NHCH2- | 2-thienyl |
| 43 | 6-quinolyl | —C(═O)NHCH2- | 2-furyl |
| 44 | 6-quinolyl | —NHC(═O)CH2- | 4-benzyloxyphenyl |
| 45 | 6-quinolyl | —NHC(═O)CH2- | phenyl |
| 46 | 6-quinolyl | —C(═O)NHCH2- | 3-thienyl |
| 47 | 6-quinolyl | —C(═O)NHCH2- | 3-fluorophenyl |
| 48 | 6-quinolyl | —C(═O)NHCH2- | 4-fluorophenyl |
| 49 | 6-quinolyl | —C(═O)NHCH2- | 3-trifluoromethylphenyl |
| 50 | 6-quinolyl | —C(═S)NHCH2- | 2-(5-phenoxy)thienyl |
| 51 | 6-quinolyl | —C(═O)NHCH2- | 2,3-dimethoxyphenyl |
| 52 | 6-quinolyl | —NHC(═O)CH2- | 2-fluorophenyl |
| 53 | 6-quinolyl | —C(═O)NHCH2- | 2-methylphenyl |
| 54 | [1.5]-naphtyridin-2-yl | —C(═O)NHCH2- | 2-(5-phenoxy)thienyl |
| 55 | 6-quinolyl | —C(═O)NHCH2- | 3-trifluoromethoxyphenyl |
| 56 | 6-quinolyl | —C(═O)NHCH2- | 4-difluoromethoxyphenyl |
| 57 | 6-quinolyl | —C(═O)NHCH2- | 4-ethoxyphenyl |
| 59 | 6-quinolyl | —C(═O)NHCH2- | 2,4-difluorophenyl |
| 60 | 6-quinolyl | —C(═O)NHCH2- | 2,5-difluorophenyl |
| 61 | 6-quinolyl | —C(═O)NHCH2- | 3,5-difluorophenyl |
| 62 | 6-quinolyl | —C(═O)NHCH2- | 2,6-difluorophenyl |
| 63 | 6-quinolyl | —C(═O)NHCH2- | 3-methoxymethylphenyl |
| 64 | [1.5]-naphtyridin-2-yl | —C(═O)NHCH2- | 2-fluoro-3-cyclopropylmethoxyphenyl |
| 65 | 6-quinolyl | —C(═O)NHCH2- | 2,3-difluorophenyl |
| 66 | 6-quinolyl | —C(═O)NHCH2- | 5-(2-benzyloxy)thiazolyl |
| 67 | 6-quinolyl | —C(═O)NHCH2- | 4-(2,4,6-trifluorobenzyloxy)phenyl |
| 68 | 6-quinolyl | —C(═O)NHCH2- | 3-benzyloxy-2-fluorophenyl |
| 69 | 6-quinolyl | —C(═O)NHCH2- | 4-(2,4-difluorobenzyloxy)phenyl |
| 70 | 6-quinolyl | —C(═O)NHCH2- | 4-methoxymethylphenyl |
| 71 | 6-quinolyl | —C(═O)NHCH2- | 3-ethoxymethylphenyl |
| 72 | 6-quinolyl | —C(═O)NHCH2- | 4-(phenoxymethyl)phenyl |
| 73 | 6-quinolyl | —C(═O)NHCH2- | 4-iso-propylphenyl |
| 74 | 6-quinolyl | —C(═O)NHCH2- | 3-difluoromethoxyphenyl |
| 75 | 6-quinolyl | —C(═O)NHCH2- | 4-methylthiophenyl |
| 76 | 6-benzothiazolyl | —C(═O)NHCH2- | 3-(3-methyl-2-butenyloxy)phenyl |
| 77 | 6-quinolyl | —C(═O)NHCH2- | 3-fluoro-4-phenoxylphenyl |
| 78 | 6-quinolyl | —C(═O)NHCH2- | 3-(2-fluorophenoxy)phenyl |
| 79 | 6-quinolyl | —C(═O)NHCH2- | 4-(3-fluorophenoxy)phenyl |
| 80 | 6-quinolyl | —C(═O)NHCH2- | 2-fluoro-4-phenoxyphenyl |
| 81 | 6-quinolyl | —C(═O)NHCH2- | 2-[5-(3-fluorobenzyl)] furyl |
| 82 | 6-quinolyl | —C(═O)NHCH2- | 3-(2-phenylethyl)phenyl |
| 83 | 7-fluoro-6-quinolyl | —C(═O)NHCH2- | phenyl |
| 84 | 7-fluoro-6-quinolyl | —C(═O)NHCH2- | 2-fluorophenyl |
| 85 | 7-fluoro-6-quinolyl | —C(═O)NHCH2- | 3-phenoxyphenyl |
| 86 | 7-fluoro-6-quinolyl | —C(═O)NHCH2- | 4-benzyloxyphenyl |
| 87 | 6-quinolyl | —C(═O)NHCH2- | 5-(1-butyl)tetrazolyl |
| 88 | 6-quinolyl | —C(═O)NHCH2- | 3-(4-methoxyphenoxy)phenyl |
| 89 | 6-quinolyl | —C(═O)NHCH2- | 4-(2,4-difluorophenoxymethyl)phenyl |
| 90 | 6-quinolyl | —C(═O)NHCH2- | 3-(3-methyl-2-butenyloxy)phenyl |
| 91 | 6-quinolyl | —C(═O)NHCH2- | 2-fluoro-3-(3-methyl-2-butenyloxy)phenyl |
| 92 | 2-methyl-6-quinolyl | —C(═O)NHCH2- | 3-phenoxyphenyl |
| 93 | 8-fluoro-6-quinolyl | —C(═O)NHCH2- | phenyl |
| 94 | 8-fluoro-6-quinolyl | —C(═O)NHCH2- | 2-fluorophenyl |
| 95 | 8-fluoro-6-quinolyl | —C(═O)NHCH2- | 3-phenoxyphenyl |
| 96 | 6-quinolyl | —C(═O)NHCH2- | 4-(3,4-difluorobenzyloxy)phenyl |
| 97 | 6-quinolyl | —C(═O)NHCH2- | 3-(2-fluorobenzyloxy)phenyl |
| 98 | 6-quinolyl | —C(═O)NHCH2- | 3-(4-fluorobenzyloxy)phenyl |
| 99 | 6-quinolyl | —C(═O)NHCH2- | 3-(3-phenylpropyloxy)phenyl |
| 100 | 6-quinolyl | —C(═O)NHCH2- | 4-(4-pyridylmethoxy)phenyl |
| 101 | 6-quinolyl | —C(═O)NHCH2- | 5-benzyloxy-2-fluorophenyl |
| 102 | 6-quinolyl | —C(═O)NHCH2- | 3-methoxyethylphenyl |
| 103 | 6-quinolyl | —C(═O)NHCH2- | 4-(2-phenylethoxy)phenyl |
| 104 | 6-quinolyl | —C(═O)NHCH2- | 2-fluoro-5-methoxyphenyl |
| 105 | 6-quinolyl | —C(═O)NHCH2- | 2-fluoro-4-methylphenyl |
| 106 | 6-quinolyl | —C(═O)NHCH2- | 3-fluoro-4-methylphenyl |
| 107 | 6-quinolyl | —C(═O)NHCH2- | 3-methyl-4-methoxyphenyl |
| 108 | 6-quinolyl | —C(═O)NHCH2- | 3-methylthiophenyl |
| 109 | 6-quinolyl | —C(═O)NHCH2- | 3-iso-propyloxyphenyl |
| 110 | 6-quinolyl | —C(═O)NHCH2- | 3-(2-methylpropyloxy)phenyl |
| 111 | 6-quinolyl | —C(═O)NHCH2- | 4-n-propyloxyphenyl |
| 112 | 6-quinolyl | —C(═O)NHCH2- | 4-n-butyloxyphenyl |
| 113 | 6-quinolyl | —C(═O)NHCH2- | 4-n-pentyloxyphenyl |
| 114 | 6-quinolyl | —C(═O)NHCH2- | 4-n-hexyloxyphenyl |
| 115 | 6-quinolyl | —C(═O)NHCH2- | 2-fluoro-4-phenoxymethylphenyl |
| 116 | [1.5]-naphtyridin-2-yl | —C(═O)NHCH2- | 2-fluoro-3-(3-methylbutyloxy)phenyl |
| 117 | 6-quinolyl | —C(═O)NHCH2- | 3-bromo-2-fluorophenyl |
| 118 | 6-quinolyl | —C(═O)NHCH2- | 3-cyclopropylphenyl |
| 119 | 6-quinolyl | —C(═O)NHCH2- | 4-chloro-2-fluorophenyl |
| 120 | 6-quinolyl | —C(═O)NHCH2- | 2-fluoro-4-pentyloxyphenyl |
| 121 | [1.5]-naphtyridin-2-yl | —C(═O)NHCH2- | 3-benzylphenyl |
| 122 | 6-quinolyl | —C(═O)NHCH2- | 3-(3-ethoxycarbonylpropyloxy)phenyl |
| 123 | 6-quinolyl | —C(═O)NHCH2- | 3-cyclobutylmethoxyphenyl |
| 124 | 6-quinolyl | —C(═O)NHCH2- | 5-ethoxy-2-fluorophenyl |
| 125 | 6-quinolyl | —C(═O)NHCH2- | 2-fluoro-5-n-propyloxyphenyl |
| 126 | 6-quinolyl | —C(═O)NHCH2- | 5-n-butyloxy-2-fluorophenyl |
| 127 | 6-quinolyl | —C(═O)NHCH2- | 2-fluoro-5-n-hexyloxyphenyl |
| 128 | 6-quinolyl | —C(═O)NHCH2- | 2-fluoro-5-phenoxyphenyl |
| 129 | 6-quinolyl | —C(═O)NHCH2- | 2-fluoro-3-phenoxylphenyl |
| 130 | 6-quinolyl | —C(═O)NHCH2- | 3-(4-methylpentyloxy)phenyl |
| 131 | 6-quinolyl | —C(═O)NHCH2- | 2-fluoro-3-hydroxyphenyl |
| 132 | [1.5]-naphtyridin-2-yl | —C(═O)NHCH2- | 4-benzylphenyl |
| 133 | [1.5]-naphtyridin-2-yl | —C(═O)NHCH2- | 4-(2-phenylethyl)phenyl |
| 134 | [1.5]-naphtyridin-2-yl | —C(═O)NHCH2- | 3-fluorophenyl |
| 135 | 6-quinolyl | —C(═O)NHCH2- | 2-fluor-3-cyclopropylmethoxyphenyl |
| 136 | 6-quinolyl | —C(═O)NHCH2- | 2-fluoro-3-(3-phenylpropyloxy)phenyl |
| 137 | 6-quinolyl | —C(═O)NHCH2- | 4-(4-fluorobenzyloxy)-2-fluorophenyl |
| 138 | [1.5]-naphtyridin-2-yl | —C(═O)NHCH2- | 4-fluoro phenyl |
| 139 | 6-quinolyl | —C(═O)NHCH2- | 4-(4-methylphenoxy)phenyl |
| 140 | 6-quinolyl | —C(═O)NHCH2- | 3-(3-methylphenoxy)phenyl |
| 141 | 6-quinolyl | —C(═O)NHCH2- | 3-(4-phenylbutyloxy)phenyl |
| 142 | 6-quinolyl | —C(═O)NHCH2- | 4-fluoro-3-methoxyphenyl |
| 143 | 6-quinolyl | —C(═O)NHCH2- | 2-chloro-5-thienyl |
| 144 | 6-quinolyl | —C(═O)NHCH2- | 3-(3-chroro-2-propenyloxy)phenyl |
| 145 | 6-quinolyl | —C(═O)NHCH2- | 5-methyl-2-thienyl |
| 146 | 6-quinolyl | —C(═O)NHCH2- | 3-(3,3-dichroro-2-propenyloxy)phenyl |
| 147 | 6-quinolyl | —C(═O)NHCH2- | 3-(2-chroro-2-propenyloxy)phenyl |
| 148 | 6-quinolyl | —C(═O)NHCH2- | 5-methoxy-2-thienyl |
| 149 | 6-quinolyl | —C(═O)NHCH2- | 3-(2-tetrahydrofuryl)methoxyphenyl |
| 150 | 6-quinolyl | —C(═O)NHCH2- | 3-(3-cyanopropyloxy)phenyl |
| 151 | 6-quinolyl | —C(═O)NHCH2- | 3,5-dimethoxyphenyl |
| 152 | [1.5]-naphtyridin-2-yl | —C(═O)NHCH2- | 2,5-difluorophenyl |
| 153 | 6-benzothiazolyl | —C(═O)NHCH2- | 2-fluorophenyl |
| 154 | 6-quinolyl | —C(═O)NHCH2- | 2-benzyl-5-tetrazolyl |
| 155 | 6-quinolyl | —C(═O)NHCH2- | 2-fluoro-3-(2-methylpropyloxy)phenyl |
| 156 | 6-quinolyl | —C(═O)NHCH2- | 3-Iodophenyl |
| 157 | 6-quinolyl | —C(═O)NHCH2- | 2-n-butyloxy-5-thiazolyl |
| 158 | 6-quinolyl | —C(═O)NHCH2- | 2-fluoro-3-(4-phenylbutoxy)phenyl |
| 159 | 6-quinolyl | —C(═O)NHCH2- | 3-ethynylphenyl |
| 160 | 6-quinolyl | —C(═O)NHCH2- | 2-fluoro-3-methylphenyl |
| 161 | 6-quinolyl | —C(═O)NHCH2- | 3-[2-(2-methoxyethoxy)ethoxy]phenyl |
| 162 | 6-quinolyl | —C(═O)NHCH2- | 2-fluoro-3-[2-(2- |
| | | | methoxyethoxy)ethoxy]phenyl |
| 163 | 6-quinolyl | —C(═O)NHCH2- | 3-ethynyl-2-fluorophenyl |
| 164 | 6-quinolyl | —C(═O)NHCH2- | 3-(3-methoxyphenoxy)phenyl |
| 165 | 6-quinolyl | —C(═O)NHCH2- | 2-fluoro-3-(6-methoxyhexyloxy)phenyl |
| 166 | 6-quinolyl | —C(═S)NHCH2- | 2-fluoro-3-methoxyphenyl |
| 167 | 6-quinolyl | —C(═O)NHCH2- | 2-fluoro-3-(3,3-dichloro-2- |
| | | | propenyloxy)phenyl |
| 168 | 6-quinolyl | —C(═O)NHCH2- | 2-fluoro-3-(3-chloro-2-propenyloxy)phenyl |
| 169 | 6-quinolyl | —C(═O)NHCH2- | 2-fluoro-3-(2-chloro-2-propenyloxy)phenyl |
| 170 | 6-quinolyl | —C(═O)NHCH2- | 4-methoxyphenyl |
| 171 | 6-quinolyl | —C(═O)NHCH2- | 1-(3-fluorobenzyl)-3-prrolyl |
| 172 | 6-quinolyl | —C(═O)NHCH2- | 4-(4-fluorophenoxy)phenyl |
| 173 | 6-quinolyl | —C(═O)NHCH2- | 4-fluoro-3-phenoxyphenyl |
| 174 | 6-quinolyl | —C(═O)NHCH2- | 4-(4-fluorobenzyloxy)phenyl |
| 175 | 6-benzothiazolyl | —C(═O)NHCH2- | 2-fluorophenyl |
| 176 | 2-methyl-6-benzothiazolyl | —C(═O)NHCH2- | phenyl |
| 177 | 2-methyl-6-benzothiazolyl | —C(═O)NHCH2- | 2-fluorophenyl |
| 178 | 6-benzothiazolyl | —C(═O)NHCH2- | 3-(2-fluorophenoxy)phenyl |
| 179 | 6-benzothiazolyl | —C(═O)NHCH2- | 3-(3-fluorophenoxy)phenyl |
| 180 | 6-benzothiazolyl | —C(═O)NHCH2- | 3-(4-fluorophenoxy)phenyl |
| 181 | 6-benzothiazolyl | —C(═O)NHCH2- | 3-n-pentylphenyl |
| 182 | 6-benzothiazolyl | —C(═O)NHCH2- | 4-n-pentylphenyl |
| 183 | 6-benzothiazolyl | —C(═O)NHCH2- | 4-fluoro-3-phenoxyphenyl |
| 184 | 6-benzothiazolyl | —C(═O)NHCH2- | 2-[5-(3-cyanophenoxy)]thienyl |
| 185 | 6-benzothiazolyl | —C(═O)NHCH2- | 3-methoxyphenyl |
| 186 | 6-benzothiazolyl | —C(═O)NHCH2- | 2-[5-(3-fluorobenzyl)]furyl |
| 187 | 6-benzothiazolyl | —C(═O)NHCH2- | 2-methylphenyl |
| 188 | 6-benzothiazolyl | —C(═O)NHCH2- | 3-methylphenyl |
| 189 | 6-benzothiazolyl | —C(═O)NHCH2- | 4-methylphenyl |
| 190 | 6-benzothiazolyl | —C(═O)NHCH2- | 3-hydroxyphenyl |
| 191 | 6-benzothiazolyl | —C(═O)NHCH2- | 3-ethoxylphenyl |
| 192 | 6-benzothiazolyl | —C(═O)NHCH2- | 3-n-propyloxyphenyl |
| 193 | 6-benzothiazolyl | —C(═O)NHCH2- | 3-n-butyloxyphenyl |
| 194 | 6-benzothiazolyl | —C(═O)NHCH2- | 3-n-pentyloxyphenyl |
| 195 | 6-benzothiazolyl | —C(═O)NHCH2- | 3-n-hexyloxyphenyl |
| 196 | 6-benzothiazolyl | —C(═O)NHCH2- | 3-n-heptyloxyphenyl |
| 197 | 6-benzothiazolyl | —C(═O)NHCH2- | 3-fluorophenyl |
| 198 | 6-benzothiazolyl | —C(═O)NHCH2- | 4-fluorophenyl |
| 199 | 6-benzothiazolyl | —NHC(═O)CH2- | 4-phenoxyphenyl |
| 200 | 6-quinolyl | —C(═O)NHCH2- | 3-(ethoxyiminomethyl)phenyl |
| 201 | 6-benzothiazolyl | —NHC(═O)CH2- | 3-benzyloxyphenyl |
| 202 | 6-quinolyl | —C(═S)NHCH2- | 2-fluoro-3-(3-pent-4-ynyloxy)phenyl |
| 203 | 6-benzothiazolyl | —C(═O)NHCH2- | 2-[5-(3-chlorophenoxy)]thienyl |
| 204 | 6-quinolyl | —C(═S)NHCH2- | 4-benzyloxyphenyl |
| 205 | [1.5]-naphtyridin-2-yl | —C(═S)NHCH2- | 2-fluoro-3-(3-pent-4-ynyloxy)phenyl |
| 206 | 6-quinolyl | —NHC(═O)CH2- | 2-fluoro-3-methoxyphenyl |
| 207 | [1.5]-naphtyridin-2-yl | —C(═O)NHCH2- | phenyl |
| 208 | 6-benzothiazolyl | —C(═O)NHCH2- | 3-(2-methylpropyl)phenyl |
| 209 | [1.5]-naphtyridin-2-yl | —C(═O)NHCH2- | 4-phenoxyphenyl |
| 210 | 6-benzothiazolyl | —C(═O)NHCH2- | 3-(2-methylphenoxy)phenyl |
| 211 | 6-benzothiazolyl | —C(═O)NHCH2- | 2-fluoro-3-hydroxyphenyl |
| 212 | 6-benzothiazolyl | —C(═O)NHCH2- | 3-(3-methylphenoxy)phenyl |
| 213 | 6-benzothiazolyl | —C(═O)NHCH2- | 3-(4-methylphenoxy)phenyl |
| 214 | 6-benzothiazolyl | —C(═O)NHCH2- | 2-fluoro-3-(3-methylbutyloxy)phenyl |
| 215 | 6-benzothiazolyl | —C(═O)NHCH2- | 2-fluoro-3-n-propylphenyl |
| 216 | 6-benzothiazolyl | —C(═O)NHCH2- | 2-fluoro-3-n-butylphenyl |
| 217 | 6-benzothiazolyl | —C(═O)NHCH2- | 2-fluoro-3-n-pentylphenyl |
| 218 | 6-benzothiazolyl | —C(═O)NHCH2- | 2-fluoro-3--n-hexylphenyl |
| 219 | 6-benzothiazolyl | —C(═O)NHCH2- | 3-(3-methylbutyloxy)phenyl |
| 220 | 6-benzothiazolyl | —C(═O)NHCH2- | 2-fluoro-3-(3-methyl-2-butenyloxy)phenyl |
| 221 | 6-benzothiazolyl | —C(═O)NHCH2- | 2-fluoro-3-(2-methyl-1-propyloxy)phenyl |
| 222 | 6-benzothiazolyl | —NHC(═O)CH2- | 4-methoxyphenyl |
| 223 | 6-benzothiazolyl | —NHC(═O)CH2- | 3-methylphenyl |
| 224 | 6-benzothiazolyl | —NHC(═O)CH2- | 4-methylphenyl |
| 225 | 6-benzothiazolyl | —NHC(═O)CH2- | 2-fluorophenyl |
| 226 | 6-benzothiazolyl | —NHC(═O)CH2- | 3-fluorophenyl |
| 227 | 6-benzothiazolyl | —NHC(═O)CH2- | 4-fluorophenyl |
| 228 | [1.5]-naphtyridin-2-yl | —C(═S)NHCH2- | 2-fluoro-3-methoxyphenyl |
| 229 | 6-benzothiazolyl | —NHC(═O)CH2- | 3-(3-methyl-2-butenyloxy)phenyl |
| 230 | 6-benzothiazolyl | —NHC(═O)CH2- | 3-(3-methylbutyloxy)phenyl |
| 231 | 6-quinolyl | —NHC(═S)CH2- | 3-phenoxyphenyl |

The present compound can be produced by a production method described in the pamphlet of International Patent Application No. WO2005/033079 (Patent Document 1), production methods described in the after-mentioned production examples, etc.

In the case of the agricultural composition of the present invention, in general, the present compound is mixed with a liquid carrier, a solid carrier, a gas carrier, a surfactant, etc. Thereafter, formulation auxiliary agents such as a binder or a thickener are added thereto, as necessary, so that the mixture can be formulated into a wettable powder, a granulated wettable powder, a flowable agent, a granule, a dry flowable agent, an emulsion, an aqueous liquid agent, an oil agent, a smoking agent, an aerosol, a microcapsule, etc., and the thus produced formulations can be then used. The present compound is comprised in such a formulation at a weight ratio generally between 0.1% and 99%, and preferably between 0.2% and 90%.

Examples of a liquid carrier used in formulation include water, alcohols (e.g. methanol, ethanol, 1-propanol, 2-propanol, ethylene glycol, etc.), ketones (e.g. acetone, methyl ethyl ketone, etc.), ethers (e.g. dioxane, tetrahydrofuran, ethylene glycol monomethyl ether, diethylene glycol monomethyl ether, propylene glycol monomethyl ether, etc.), aliphatic hydrocarbons (e.g. hexane, octane, cyclohexane, coal oil, fuel oil, machine oil, etc.), aromatic hydrocarbons (e.g. benzene, toluene, xylene, solvent naphtha, methylnaphthalene, etc.), halogenated hydrocarbons (e.g. dichloromethane, chloroform, carbon tetrachloride, etc.), acid amides (e.g. dimethylformamide, dimethylacetamide, N-methylpyrrolidone, etc.), esters (e.g. ethyl acetate, butyl acetate, fatty acid glycerin ester, etc.), and nitrites (e.g. acetonitrile, propionitrile, etc.). Two or more types of such liquid carriers can be mixed at an appropriate ratio and can be then used.

Examples of a solid carrier used in formulation include vegetable powders (e.g. soybean powder, tobacco powder, wheat flour, wood flour, etc.), mineral powders (e.g. clays such as kaoline, bentonite, acid clay, or clay; talcs such as talcum powder or pyrophyllite powder; silicas such as diatomaceous earth or mica powder; etc.), alumina, sulfur powder, activated carbon, sugars (e.g. lactose, glucose, etc.), inorganic salts (e.g. calcium carbonate, sodium bicarbonate, etc.), and a glass hollow body (produced by subjecting a natural holohyaline to a burning process and then encapsulating air bubbles therein). Two or more types of such solid carriers can be mixed at an appropriate ratio and can be then used. Such liquid carrier or solid carrier is used at a ratio generally between approximately 1% to 99% by weight, and preferably between approximately 10% and 99% by weight, based on the total weight of a formulation.

As an emulsifier, a dispersant, a spreader, a penetrant, a moisturizer, or the like used in the formulation, a surfactant is generally used. Examples of such a surfactant include: anionic surfactants such as alkyl sulfate ester salts, alkylaryl sulfonates, dialkyl sulfosuccinates, polyoxyethylene alkyl aryl ether phosphates, lignosulfonates, or naphthalene sulfonate formaldehyde polycondensates; and nonionic surfactants such as a polyoxyethylene alkyl ether, a polyoxyethylene alkyl aryl ether, a polyoxyethylene alkyl polyoxy propylene block copolymer, or a sorbitan fatty acid ester. These surfactants can also be used in combination of two or more types. Such a surfactant is used at a ratio generally between 0.1% and 50% by weight, and preferably between approximately 0.1% and 25% by weight, based on the total weight of a formulation.

Examples of a binder or a thickener include dextrin, sodium salts of carboxymethyl cellulose, a polycarboxylic acid polymer, polyvinylpyrrolidone, polyvinyl alcohol, sodium lignosulfonate, calcium lignosulfonate, sodium polyacrylate, gum Arabic, sodium alginate, mannitol, sorbitol, bentonite mineral matter, polyacrylic acid and a derivative thereof, sodium salts of carboxymethyl cellulose, white carbon, and natural sugar derivatives (e.g. xanthan gum, Cyamoposis Gum, etc.)

Moreover, the agricultural composition of the present invention can be mixed with other types of fungicides, insecticides, acaricides, nematicides, herbicides, plant growth regulators, fertilizers, or soil improvers, and they can be used. Otherwise, the present agricultural composition can be used together with the aforementioned agents, without mixing them.

Examples of such other fungicides include: azoles such as propiconazole, prothioconazole, triadimenol, prochloraz, penconazole, tebuconazole, flusilazole, diniconazole, bromuconazole, epoxiconazole, difenoconazole, cyproconazole, metconazole, triflumizole, tetraconazole, microbutanil, fenbuconazole, hexaconazole, fluquinconazole, triticonazole, bitertanol, imazalil, flutriafol, simeconazole, or ipconazole; cyclic amines such as fenpropimorph, tridemorph, or fenpropidin; benzimidazoles such as carbendazim, benomyl, thiabendazole, or thiophanate-methyl; procymidone; cyprodinil; pyrimethanil; diethofencarb; thiuram; fluazinam, mancozeb; iprodione; vinclozolin, chlorothalonil; captan; mepanipyrim; fenpiclonil; fludioxonil; dichlofluanid; folpet; kresoxim-methyl; azoxystrobin; trifloxystrobin; fluoxastrobin; picoxystrobin; pyraclostrobin; dimoxystrobin; pyribencarb; metominostrobin; enestrobin; spiroxamine; quinoxyfen; fenhexamid; famoxadone; fenamidone; zoxamide; etaboxam; amisulbrom; iprovalicarb; benthiavalicarb; cyazofamid; mandipropamid; boscalid; penthiopyrad; metrafenone; fluopyram; bixafen; cyflufenamid; proquinazid; orysastrobin; furametpyr; thifluzamide; mepronil; flutolanil; flusulfamide; fluopicolide; metalaxyl M; kiralaxyl; fosetyl; cymoxanil; pencycuron; trichlofos methyl; carpropamid; diclocymet; fenoxanil; tricyclazole; pyroquilon; probenazole; isotianil; tiadinil; tebufloquine; flubuquine; fluthianil; diclomezine; kasugamycin; ferimzone; fthalide; validamycin; hydroxyisoxazole; iminoctadin-acetate; isoprothiolane; oxolinic acid; oxytetracycline; streptomycin; basic copper chloride; cupric hydroxide; basic copper sulfate; organic copper; and sulfur.

Examples of other insecticides include the following compounds:

### (1) Organophosphorus Compounds

acephate, Aluminium phosphide, butathiofos, cadusafos, chlorethoxyfos, chlorfenvinphos, chlorpyrifos, chlorpyrifos-methyl, cyanophos: CYAP, diazinon, dichlorodiisopropyl ether, dichlofenthion: ECP, dichlorvos: DDVP, dimethoate, dimethylvinphos, disulfoton, EPN, ethion, ethoprophos, etrimfos, fenthion: MPP, fenitrothion: MEP, fosthiazate, formothion, Hydrogen phosphide, isofenphos, isoxathion, malathion, mesulfenfos, methidathion: DMTP, monocrotophos, naled: BRP, oxydeprofos: ESP, parathion, phosalone, phosmet: PMP, pirimiphos-methyl, pyridafenthion, quinalphos, phenthoate: PAP, profenofos, propaphos, prothiofos, pyraclorfos, salithion, sulprofos, tebupirimfos, temephos, tetrachlorvinphos, terbufos, thiometon, trichlorphon: DEP, vamidothion, phorate, cadusafos and the like;

### (2) Carbamate Compounds

alanycarb, bendiocarb, benfuracarb, BPMC, carbaryl, carbofuran, carbosulfan, cloethocarb, ethiofencarb, fenobucarb, fenothiocarb, fenoxycarb, furathiocarb, isoprocarb: MIPC, metolcarb, methomyl, methiocarb, NAC, oxamyl, pirimicarb, propoxur: PHC, XMC, thiodicarb, xylylcarb, aldicarb and the like;

### (3) Synthetic Pyrethroid Compounds

acrinathrin, allethrin, benfluthrin, beta-cyfluthrin, bifenthrin, cycloprothrin, cyfluthrin, cyhalothrin, cypermethrin, deltamethrin, esfenvalerate, ethofenprox, fenpropathrin, fenvalerate, flucythrinate, flufenoprox, flumethrin, fluvalinate, halfenprox, imiprothrin, permethrin, prallethrin, pyrethrins, resmethrin, sigma-cypermethrin, silafluofen, tefluthrin, tralomethrin, transfluthrin, tetramethrin, phenothrin, cyphenothrin, alpha-cypermethrin, zeta-cypermethrin, lambda-cyhalothrin, furamethrin, tau-fluvalinate, 2,3,5,6-tetrafluoro-4-(methoxymethyl)benzil (EZ)-(1RS,3RS;1RS,3SR)-2,2-dimethyl-3-prop-1-enylcyclopropanecarboxylate, 2,3,5,6-tetrafluoro-4-methylbenzyl (EZ)-(1RS,3RS;1RS,3SR)-2,2-dimethyl-3-prop-1-enylcyclopropanecarboxylate, 2,3,5,6-tetrafluoro-4-(methoxymethyl)benzil (1RS,3RS;1RS,3SR)-2,2-dimethyl-3-(2-methyl-1-propenyl)cyclopropanecarboxylate and the like;

### (4) Nereistoxin Compounds

cartap, bensultap, thiocyclam, monosultap, bisultap and the like;

### (5) Neonicotinoid Compounds

imidacloprid, nitenpyram, acetamiprid, thiamethoxam, thiacloprid, dinotefuran, clothianidin and the like;

### (6) Benzoylurea Compounds

chlorfluazuron, bistrifluoron, diafenthiuron, diflubenzuron, fluazuron, flucycloxuron, flufenoxuron, hexaflumuron, lufenuron, novaluron, noviflumuron, teflubenzuron, triflumuron, triazuron and the like;

### (7) Phenylpyrazole Compounds

acetoprole, ethiprole, fipronil, vaniliprole, pyriprole, pyrafluprole and the like;

### (8) Bt Toxin Insecticides

Fresh spores derived from Bacillus thuringiensis, crystalline toxins generated therefrom, and the mixtures thereof;

### (9) Hydrazine Compounds

chromafenozide, halofenozide, methoxyfenozide, tebufenozide and the like;

### (10) Organochlorine Compounds

aldrin, dieldrin, dienochlor, endosulfan, methoxychlor and the like;

### (11) Natural Insecticides

machine oil, nicotine-sulfate;

### (12) Other Types of Insecticides

avermectin-B, bromopropylate, buprofezin, chlorphenapyr, cyromazine, D-D(1,3-Dichloropropene), emamectin-benzoate, fenazaquin, flupyrazofos, hydroprene, methoprene, indoxacarb, metoxadiazone, milbemycin-A, pymetrozine, pyridalyl, pyriproxyfen, spinosad, sulfluramid, tolfenpyrad, triazamate, flubendiamide, lepimectin, Arsenic acid, benclothiaz, Calcium cyanamide, Calcium polysulfide, chlordane, DDT, DSP, flufenerim, flonicamid, flurimfen, formetanate, metam-ammonium, metam-sodium, Methyl bromide, nidinotefuran, Potassium oleate, protrifenbute, spiromesifen, Sulfur, metaflumizone, spirotetramat, pyrifluquinazone, spinetoram, chlorantraniliprole.

Examples of other types of acaricides (acarcidal active ingredients) include

acequinocyl, amitraz, benzoximate, bifenaate, bromopropylate, chinomethionat, chlorobenzilate, CPCBS (chlorfenson), clofentezine, cyflumetofen, kelthane (dicofol), etoxazole, fenbutatin oxide, fenothiocarb, fenpyroximate, fluacrypyrim, fluproxyfen, hexythiazox, propargite: BPPS, polynactins, pyridaben, Pyrimidifen, tebufenpyrad, tetradifon, spirodiclofen, spiromesifen, spirotetramat, amidoflumet, cyenopyrafen and the like.

Examples of such other types of nematicides (nematicidal active ingredients) include DCIP, fosthiazate, levamisol hydrochloride, methylisothiocyanate; morantel tartarate, and imicyafos.

The weight ratio between the agricultural composition of the present invention and a fungicide mixed with or used together with the aforementioned agricultural composition is generally between 1:0.01 and 1:100, and preferably between 1:0.1 and 1:10, in terms of active ingredient.

The weight ratio between the agricultural composition of the present invention and an insecticide mixed with or used together with the aforementioned agricultural composition is generally between 1:0.01 and 1:100, and preferably between 1:0.1 and 1:10, in terms of active ingredient.

The weight ratio between the agricultural composition of the present invention and an acaricide mixed with or used together with the aforementioned agricultural composition is generally between 1:0.01 and 1:100, and preferably between 1:0.1 and 1:10, in terms of active ingredient.

The weight ratio between the agricultural composition of the present invention and a nematicide mixed with or used together with the aforementioned agricultural composition is generally between 1:0.01 and 1:100, and preferably between 1:0.1 and 1:10, in terms of active ingredient.

The weight ratio between the agricultural composition of the present invention and a herbicide mixed with or used together with the aforementioned agricultural composition is generally between 1:0.01 and 1:100, and preferably between 1:0.1 and 1:10, in terms of active ingredient.

The weight ratio between the agricultural composition of the present invention and a plant growth regulator mixed with or used together with the aforementioned agricultural composition is generally between 1:0.00001 and 1:100, and preferably between 1:0.0001 and 1:1, in terms of active ingredient.

The weight ratio between the agricultural composition of the present invention and a fertilizer or soil improver mixed with or used together with the aforementioned agricultural composition is generally between 1:0.1 and 1:1000, and preferably between 1:1 and 1:200, in terms of active ingredient.

The agricultural composition of the present invention can be used to control or prevent diseases caused by plant pathogenic microbes except for Aspergillus in agricultural lands such as fields, paddy fields, lawns, or orchards.

The type of a method of applying the agricultural composition of the present invention is not particularly limited, as long as the agricultural composition of the present invention can be substantially applied by the method. Examples of such an application method: include application of the present agricultural composition to the plant bodies of useful plants, such as foliage application; application of the present agricultural composition to a field in which useful plants are planted or are to be planted, such as a soil treatment; and application of the present agricultural composition to seeds, such as seed sterilization.

An effective amount of the agricultural composition of the present invention applied to useful crops differs depending on a climatic condition, a dosage form, an application period, an application method, an application site, a target disease, target crops, etc. In terms of the present compound contained in the agricultural composition of the present invention, the effective amount is generally between 1 and 500 g, and preferably 2 and 200 g, per 10 ares. An emulsion, a wettable powder, a suspension, etc. are generally diluted with water for application. In such a case, the concentration of the present compound after dilution is generally between 0.0005% and 2% by weight, and preferably between 0.005% and 1% by weight. On the other hand, a powder, a granule, etc. are directly applied without being diluted. When the agricultural composition of the present invention is applied to seeds, the present compound contained in the agricultural composition of the present invention hydrant is applied within a range generally between 0.001 and 100 g, and preferably between 0.01 and 50 g, per kg of seeds.

The agricultural composition of the present invention can control or prevent diseases caused by plant pathogenic microbes in farmlands for cultivating the following useful crops.

Examples of such useful crops are as follows.

Crops: corn, rice, wheat, barley, rye, oat, sorghum, cotton, soybean, peanut, buckwheat, beet, rapeseed, sunflower, sugar cane, tobacco, etc.; vegetables: solanaceous vegetables (eggplant, tomato, pimento, pepper, potato, etc.), cucurbitaceous vegetables (cucumber, pumpkin, zucchini, water melon, melon, etc.), cruciferous vegetables (Japanese radish, white turnip, horseradish, kohlrabi, Chinese cabbage, cabbage, leaf mustard, broccoli, cauliflower, etc.), asteraceous vegetables (burdock, crown daisy, artichoke, lettuce, etc.), liliaceous vegetables (green onion, onion, garlic, and asparagus), ammiaceous vegetables (carrot, parsley, celery, parsnip, etc.), chenopodiaceous vegetables (spinach, Swiss chard, etc.), lamiaceous vegetables (Perilla frutescens, mint, basil, etc.), strawberry, sweet potato, Dioscorea japonica, colocasia, etc., flowers, foliage plants; fruits: pomaceous fruits (apple, pear, Japanese pear, Chinese quince, quince, etc.), stone fleshy fruits (peach, plum, nectarine, Prunus mume, cherry fruit, apricot, prune, etc.), citrus fruits (Citrus unshiu, orange, lemon, lime, grapefruit, etc.), nuts (chestnut, walnuts, hazelnuts, almond, pistachio, cashew nuts, macadamia nuts, etc.), berrys (blueberry, cranberry, blackberry, raspberry, etc.), grape, kaki fruit, olive, Japanese plum, banana, coffee, date palm, coconuts, trees other than fruit trees; tea, mulberry, flowering plant, roadside trees (ash, birch, dogwood, Eucalyptus, Ginkgo biloba, lilac, maple, Quercus, poplar, Judas tree, Liquidambar formosana, plane tree, zelkova, Japanese arborvitae, fir wood, hemlock, juniper, Pinus, Picea, and Taxus cuspidate), etc.

The aforementioned “useful crops” include crops, to which resistance to HPPD inhibitors such as isoxaflutole, ALS inhibitors such as imazethapyr or thifensulfuron-methyl, EPSP synthetase inhibitors, glutamine synthetase inhibitors, and herbicides such as bromoxynil, has been imparted by a classical breeding method or genetic recombination.

Examples of “useful crops” to which resistance is imparted by a classical breeding method include Clearfield (registered trademark) canola that is resistant to imidazolinone herbicides such as imazethapyr, and STS soybean that is resistant to sulfonylurea ALS inhibitory herbicides such as thifensulfuron-methyl. In addition, examples of “useful crops” to which resistance is imparted by genetic recombination include corn varieties resistant to glyphosate or glufosinate. Such corn varieties have already been on the market with product names such as “RoundupReady (registered trademark) and “LibertyLink (registered trademark).”

The aforementioned “useful crops” include genetically recombinant crops produced using such genetic recombination techniques, which are able to synthesize selective toxins as known in genus Bacillus.

Examples of toxins expressed in such genetically recombinant crops include: insecticidal proteins derived from Bacillus cereus or Bacillus popilliae; δ-endotoxins such as Cry1Ab, Cry1Ac, Cry1F, Cry1Fa2, Cry2Ab, Cry3A, Cry3Bb1 or Cry9, derived from Bacillus thuringiensis; insecticidal proteins such as VIP1, VIP2, VIP3, or VIP3A; insecticidal proteins derived from nematodes; toxins generated by animals, such as scorpion toxin, spider toxin, bee toxin, or insect-specific neurotoxins; mold fungi toxins; plant lectin; agglutinin; protease inhibitors such as a trypsin inhibitor, a serine protease inhibitor, patatin, cystatin, or a papain inhibitor; ribosome-inactivating proteins (RIP) such as lycine, corn-RIP, abrin, luffin, saporin, or briodin; steroid-metabolizing enzymes such as 3-hydroxysteroid oxidase, ecdysteroid-UDP-glucosyl transferase, or cholesterol oxidase; an ecdysone inhibitor; HMG-COA reductase; ion channel inhibitors such as a sodium channel inhibitor or calcium channel inhibitor; juvenile hormone esterase; a diuretic hormone receptor; stilbene synthase; bibenzyl synthase; chitinase; and glucanase.

Moreover, toxins expressed in such genetically recombinant crops also include: hybrid toxins of δ-endotoxin proteins such as Cry1Ab, Cry1Ac, Cry1F, Cry1Fa2, Cry2Ab, Cry3A, Cry3Bb1 or Cry9, and insecticidal proteins such as VIP1, VIP2, VIP3 or VIP3A; partially deleted toxins; and modified toxins. Such hybrid toxins are produced from a new combination of the different domains of such proteins, using a genetic recombination technique. As a partially deleted toxin, Cry1Ab comprising a deletion of a portion of an amino acid sequence has been known. A modified toxin is produced by substitution of one or multiple amino acids of natural toxins.

Examples of such toxins and recombinant plants capable of synthesizing such toxins are described in EP-A-0 374 753, WO 93/07278, WO 95/34656, EP-A-0 427 529, EP-A-451 878, WO 03/052073, etc.

Toxins contained in such recombinant plants are able to impart resistance particularly to insect pests belonging to Coleoptera, Diptera and Lepidoptera, to the plants.

Furthermore, genetically recombinant plants, which comprise one or multiple insecticidal pest-resistant genes and which express one or multiple toxins, have already been known, and some of such genetically recombinant plants have already been on the market. Examples of such genetically recombinant plants include YieldGard (registered trademark) (a corn variety for expressing Cry1Ab toxin), YieldGard Rootworm (registered trademark) (a corn variety for expressing Cry3Bb1 toxin), YieldGard Plus (registered trademark) (a corn variety for expressing Cry1Ab and Cry3Bb1 toxins), Herculex I (registered trademark) (a corn variety for expressing phosphinotricine N-acetyl transferase (PAT) so as to impart resistance to Cry1Fa2 toxin and gluphosinate), NuCOTN33B (a cotton variety for expressing Cry1Ac toxin), Bollgard I (registered trademark) (a cotton variety for expressing Cry1Ac toxin), Bollgard II (registered trademark) (a cotton variety for expressing Cry1Ac and Cry2Ab toxins), VIPCOT (registered trademark) (a cotton variety for expressing VIP toxin), NewLeaf (registered trademark) (a potato variety for expressing Cry3A toxin), NatureGard (registered trademark) Agrisure (registered trademark) GT Advantage (GA21 glyphosate-resistant trait), Agrisure (registered trademark) CB Advantage (Bt11 corn borer (CB) trait), and Protecta (registered trademark).

The aforementioned “useful crops” also include crops produced using a genetic recombinant technique, which have ability to generate antipathogenic substances having selective action.

A PR protein and the like have been known as such antipathogenic substances (PRPs, EP-A-0 392 225). Such antipathogenic substances and genetically recombinant crops that generate them are described in EP-A-0 392 225, WO 95/33818, EP-A-0 353 191, etc.

Examples of such antipathogenic substances expressed in genetically recombinant crops include: ion channel inhibitors such as a sodium channel inhibitor or a calcium channel inhibitor (KP1, KP4 and KP6 toxins, etc., which are produced by viruses, have been known); stilbene synthase; bibenzyl synthase; chitinase; glucanase; a PR protein; and antipathogenic substances generated by microorganisms, such as a peptide antibiotic, an antibiotic having a hetero ring, a protein factor associated with resistance to plant diseases (which is called a plant disease-resistant gene and is described in WO 03/000906).

Preferred examples of plant pathogenic microbes that can be controlled by the present invention are plant pathogenic filamentous fungi except for Aspergillus. More specific examples include the following plant pathogenic microbes, but examples are not limited thereto. Plant disease names and plant pathogenic microbe names will be exemplified below: blast (Magnaporthe grisea), Helminthosporium leaf spot (Cochliobolus miyabeanus), sheath blight (Rhizoctonia solani), bakanae disease (Gibberella fujikuroi) and downy mildew (Sclerophthora macrospora) of rice; powdery mildew (Erysiphe graminis), Fusarium head blight (Fusarium graminearum, F. avenacerum, F. culmorum, Microdochium nivale), rust (Puccinia striiformis, P. graminis, P. recondita, P. hordei), snow blight (Typhula sp., Micronectriella nivalis), loose smut (Ustilago tritici, U. nuda), bunt (Tilletia caries), eyespot (Pseudocercosporella herpotrichoides), scald (Rhynchosporium secalis), leaf blotch (Septoria tritici), glume blotch (Leptosphaeria nodorum), net blotch (Pyrenophora teres Drechsler), take-all (Gaeumannomyces graminis) and tan spot (Pyrenophora tritici-repentis) of barley, wheat, oats and rye; melanose (Diaporthe citri), scab (Elsinoe fawcetti) and Penicillium rot (Penicillium digitatum, P. italicum) of citrus;

blossom blight (Monilinia mali), canker (Valsa ceratosperma), powdery mildew (Podosphaera leucotricha), Alternaria leaf spot (Alternaria alternata apple pathotype), scab (Venturia inaequalis) and bitter rot (Glomerella cingulata) of apple; scab (Venturia nashicola, V. pirina), black spot (Alternaria alternata Japanese pear pathotype) and rust (Gymnosporangium haraeanum) of pear;
brown rot (Monilinia fructicola), scab (Cladosporium carpophilum) and Phomopsis rot (Phomopsis sp.) of peach;
anthracnose (Elsinoe ampelina), ripe rot (Glomerella cingulata), powdery mildew (Uncinula necator), rust (Phakopsora ampelopsidis), black rot (Guignardia bidwellii) and downy mildew (Plasmopara viticola) of grape;
anthracnose (Gloeosporium kaki) and leaf spot (Cercospora kaki, Mycosphaerella nawae) of Japanese persimmon;
anthracnose (Colletotrichum lagenarium), powdery mildew (Sphaerotheca fuliginea), gummy stem blight (Mycosphaerella melonis), Fusarium wilt (Fusarium oxysporum), downy mildew (Pseudoperonospora cubensis), Phytophthora rot (Phytophthora sp.) and damping-off (Pythium sp.) of gourd family;
early blight (Alternaria solani), leaf mold (Cladosporium fulvum) and late blight (Phytophthora infestans) of tomato;
brown spot (Phomopsis vexans) and powdery mildew (Erysiphe cichoracearum) of eggplant;
Alternaria leaf spot (Alternaria japonica), white spot (Cercosporella brassicae), clubroot (Plasmodiophora parasitica), and downy mildew (Peronospora parasitica) of Cruciferous Vegetables;
rust (Puccinia allii) of Welsh onion;
purple seed stain (Cercospora kikuchii), Sphaceloma scad (Elsinoe glycines), pod and stem blight (Diaporthe phaseolorum var. sojae) and rust (Phakopsora pachyrhizi) of soybean;
anthracnose (Colletotrichum lindemthianum) of kidney bean;
leaf spot (Cercospora personata), brown leaf spot (Cercospora arachidicola) and southern blight (Sclerotium rolfsii) of peanut;
powdery mildew (Erysiphe pisi) of garden pea;
early blight (Alternaria solani), late blight (Phytophthora infestans) and Verticillium wilt (Verticillium albo-atrum, V. dahliae, V. nigrescens) of potato;
powdery mildew (Sphaerotheca humuli) of strawberry;
net blister blight (Exobasidium reticulatum), white scab (Elsinoe leucospila), gray blight (Pestalotiopsis sp.) and anthracnose (Colletotrichum theae-sinensis) of tea;
brown spot (Alternaria longipes), powdery mildew (Erysiphe cichoracearum), anthracnose (Colletotrichum tabacum), downy mildew (Peronospora tabacina) and black shank (Phytophthora nicotianae) of tabacco;
Cercospora leaf spot (Cercospora beticola), leaf blight (Thanatephorus cucumeris), Root rot (Thanatephorus cucumeris) and Aphanomyces root rot (Aphanomyces sochlioides) of sugar beet;
black spot (Diplocarpon rosae) and powdery mildew (Sphaerotheca pannosa) of rose;
leaf blight (Septoria chrysanthemi-indici) and white rust (Puccinia horiana) of chrysanthemum;
mycelial neck rot (Botrytis cinerea, B. byssoidea, B. squamosa), gray-mold neck rot (Botrytis alli) and small sclerotial neck rot (Botrytis squamosa) of onion;
gray mold (Botrytis cinerea) and Sclerotinia rot (Sclerotinia sclerotiorum) of various crops;
Alternaria leaf spot (Alternaria brassicicola) of Japanese radish;
dollar spot (Sclerotinia homeocarpa), brown patch and large patch (Rhizoctonia solani) of turfgrass; and Sigatoka disease (Mycosphaerella fijiensis, Mycosphaerella musicola) of banana.

### EXAMPLES

Next, the present invention will be more specifically described in the following examples including production examples, formulation examples, test examples, and the like. However, these examples are not intended to limit the scope of the present invention.

### Production Example 1

0.34 g of 1-ethyl-3-(3-dimethylaminopropyl)carbodiimide hydrochloride (hereinafter abbreviated as WSC) was added to a mixture of 0.26 g of 6-quinoline carboxylic acid, 0.16 g of benzylamine and 5 ml of pyridine. The obtained mixture was stirred at a room temperature for 1.5 hours. Thereafter, water was added to the reaction mixture, followed by extraction with ethyl acetate. The organic layer was successively washed with water and a saturated sodium chloride, and it was then dried over anhydrous sodium sulfate, followed by concentration under a reduced pressure. The residue was subjected to silica gel column chromatography, so as to obtain 0.26 g of N-benzyl-6-quinoline carboxamide (1).

### N-benzyl-6-quinoline carboxamide

1H-NMR (CDCl3) δ: 4.72 (2H, d, J=5.6 Hz), 6.57 (1H, br s), 7.30-7.42 (5H, m), 7.47 (1H, dd, J=8.3, 4.1 Hz), (1H, dd, J=8.8, 2.0 Hz), 8.15 (1H, d, J=8.8 Hz), 8.23 (1H, d, J=8.3 Hz), 8.33 (1H, d, J=2.0 Hz), 8.99 (1H, dd, J=4.1, 1.7 Hz).

The products as shown in Table-2 were produced in the same manner as described in Production example 1.

TABLE-2
(NMR data)

| Structure | NMR Data |
|---|---|
| 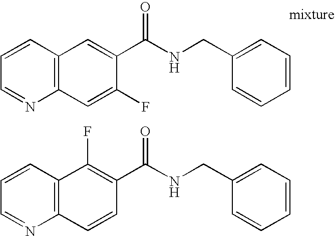 | 1H-NMR (CDCl3) δ: 4.50 (0.2H, d, J = 5.6 Hz), 4.76 (1.8H, d, J = 5.6 Hz), 7.18-7.22 (1.0H, m), 7.30-7.41 (5.0H, m), 7.46 (0.9H, dd, J = 8.3, 4.1 Hz), 7.53 (0.1H, dd, J = 8.4, 4.3 Hz), 7.80 (0.9H, d, J = 13.7 Hz), 8.00 (0.1H, d, J = 9.0 Hz), 8.29 (0.9H, d, J = 7.3 Hz), 8.37 (0.1H, t, J = 8.8 Hz), 8.48 (0.1H, d, J = 8.5 Hz), 8.73 (0.9H, d, J = 8.3 Hz), 8.99 (0.9H, dd, J = 4.3, 1.6 Hz), 9.03 (0.1H, dd, J = 4.4, 1.7 Hz). |
| 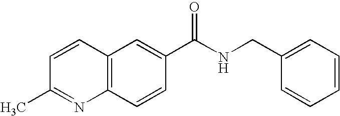 | 1H-NMR (CDCl3) δ: 2.77 (3H, s), 4.71 (2H, d, J = 5.6 Hz), 6.53 (1H, s), 7.32-7.41 (6H, m), 8.02 (1H, dd, J = 8.8, 1.7 Hz), 8.05 (1H, d, J = 8.8 Hz), 8.11 (1H, d, J = 8.3 Hz), 8.29 (1H, d, J = 1.5 Hz). |
| 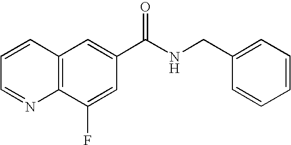 | 1H-NMR (CDCl3) δ: 4.70 (2H, d, J = 5.6 Hz), 6.60 (1H, s), 7.30-7.39 (5H, m), 7.54 (1H, dd, J = 8.4, 4.3 Hz), 7.82 (1H, dd, J = 10.9, 1.8 Hz), 8.09 (1H, s), 8.25 (1H, dt, J = 8.4, 1.5 Hz), 9.03 (1H, dd, J = 4.3, 1.6 Hz). |
| 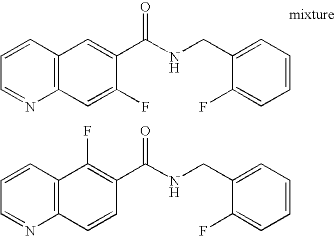 | 1H-NMR (CDCl3) δ: 4.55 (0.2H, d, J = 5.9 Hz), 4.79 (1.8H, d, J = 5.9 Hz), 7.02-7.17 (2.0H, m), 7.27-7.39 (2.0H, m), 7.43-7.48 (1.9H, m), 7.53 (0.1H, dd, J = 8.5, 4.1 Hz), 7.80 (0.9H, d, J = 13.4 Hz), 7.98 (0.1H, d, J = 9.0 Hz), 8.27 (0.9H, d, J = 8.5 Hz), 8.34 (0.1H, t, J = 8.7 Hz), 8.49 (0.1H, d, J = 9.5 Hz), 8.71 (0.9H, d, J = 8.5 Hz), 8.98 (0.9H, dd, J = 4.3, 1.6 Hz), 9.02 (0.1H, dd, J = 4.1, 1.7 Hz). |
| 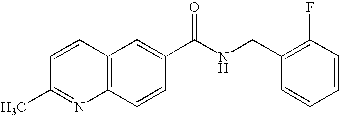 | 1H-NMR (CDCl3) δ: 2.77 (3H, s), 4.76 (2H, d, J = 5.9 Hz), 6.64 (1H, s), 7.09 (1H, t, J = 9.6 Hz), 7.14 (1H, t, J = 7.4 Hz), 7.27-7.33 (1H, m), 7.35 (1H, d, J = 8.5 Hz), 7.47 (1H, td, J = 7.6, 1.5 Hz), 8.01 (1H, dd, J = 8.8, 2.0 Hz), 8.05 (1H, d, J = 9.0 Hz), 8.11 (1H, d, J = 8.3 Hz), 8.28 (1H, d, J = 1.5 Hz). |
| 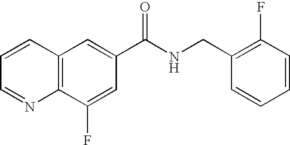 | 1H-NMR (CDCl3) δ: 4.75 (2H, d, J = 5.4 Hz), 6.71 (1H, s), 7.09 (1H, t, J = 9.3 Hz), 7.15 (1H, t, J = 7.4 Hz), 7.28-7.33 (1H, m), 7.45 (1H, t, J = 7.6 Hz), 7.54 (1H, dd, J = 8.3, 4.1 Hz), 7.80 (1H, d, J = 11.0 Hz), 8.08 (1H, s), 8.25 (1H, d, J = 8.5 Hz), 9.02-9.04 (1H, m). |
| 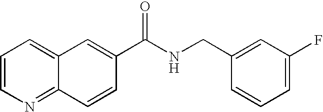 | 1H-NMR (CDCl3) δ: 4.71 (2H, d, J = 5.9 Hz), 6.73 (1H, br s), 6.98-7.02 (1H, m), 7.10 (1H, d, J = 9.5 Hz), 7.16 (1H, d, J = 7.8 Hz), 7.30-7.36 (1H, m), 7.47 (1H, dd, J = 8.3, 4.4 Hz), 8.07 (1H, dd, J = 8.8, 2.0 Hz), 8.15 (1H, d, J = 8.8 Hz), 8.22 (1H, dd, J = 8.2, 1.1 Hz), 8.34 (1H, d, J = 2.0 Hz), 8.99 (1H, dd, J = 4.4, 1.7 Hz). |
| 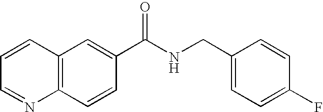 | 1H-NMR (CDCl3) δ: 4.68 (2H, d, J = 5.6 Hz), 6.65 (1H, br s), 7.03-7.08 (2H, m), 7.35-7.39 (2H, m), 7.47 (1H, dd, J = 8.3, 4.1 Hz), 8.06 (1H, dd, J = 8.8, 2.2 Hz), 8.15 (1H, d, J = 8.8 Hz), 8.23 (1H, d, J = 7.8 Hz), 8.33 (1H, d, J = 1.7 Hz), 8.99 (1H, dd, J = 4.4, 1.7 Hz). |
| 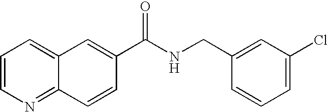 | 1H-NMR (CDCl3) δ: 4.69 (2H, d, J = 5.9 Hz), 6.70 (1H, br s), 7.26-7.31 (3H, m), 7.38 (1H, s), 7.47 (1H, dd, J = 8.3, 4.1 Hz), 8.07 (1H, dd, J = 8.8, 2.0 Hz), (1H, d, J = 8.8 Hz), 8.23 (1H, dd, J = 8.4, 0.9 Hz), 8.34 (1H, d, J = 2.0 Hz), 8.99 (1H, dd, J = 4.4, 1.7 Hz). |
| 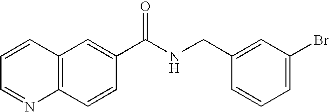 | 1H-NMR (DMSO-D6) δ: 4.55 (2H, d, J = 5.9 Hz), 7.32 (1H, t, J = 7.6 Hz), 7.39 (1H, d, J = 7.6 Hz), 7.47 (1H, d, J = 7.8 Hz), 7.57 (1H, s), 7.62 (1H, dd, J = 8.0, 3.9 Hz), 8.11 (1H, d, J = 8.8 Hz), 8.22 (1H, d, J = 9.0 Hz), 8.49 (1H, d, J = 8.3 Hz), 8.57 (1H, s), 8.99-9.00 (1H, m), 9.34 (1H, t, J = 5.5 Hz). |
| 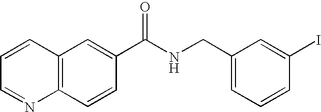 | 1H-NMR (CDCl3) δ: 4.65 (2H, d, J = 5.9 Hz), 6.77 (1H, s), 7.09 (1H, t, J = 7.7 Hz), 7.36 (1H, d, J = 7.6 Hz), 7.47 (1H, dd, J = 8.3, 4.1 Hz), 7.64 (1H, d, J = 8.0 Hz), 7.73 (1H, s), 8.07 (1H, dd, J = 8.8, 2.0 Hz), 8.15 (1H, d, J = 8.8 Hz), 8.22 (1H, dd, J = 8.2, 1.1 Hz), 8.33 (1H, d, J = 2.0 Hz), 8.99 (1H, dd, J = 4.1, 1.7 Hz). |
| 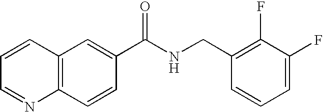 | 1H-NMR (CDCl3) δ: 4.78 (2H, d, J = 5.9 Hz), 6.83 (1H, br s), 7.05-7.13 (2H, m), 7.21-7.26 (1H, m), 7.47 (1H, dd, J = 8.4, 4.3 Hz), 8.06 (1H, dd, J = 8.9, 1.8 Hz), 8.15 (1H, d, J = 8.8 Hz), 8.23 (1H, d, J = 8.3 Hz), 8.32 (1H, s), 8.98 (1H, dd, J = 4.1, 1.7 Hz). |
| 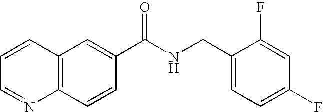 | 1H-NMR (CDCl3) δ: 4.71 (2H, d, J = 5.9 Hz), 6.77 (1H, br s), 6.81-6.90 (2H, m), 7.43-7.49 (2H, m), 8.04 (1H, dd, J = 8.8, 2.0 Hz), 8.14 (1H, d, J = 8.8 Hz), 8.22 (1H, dd, J = 8.4, 1.1 Hz), 8.31 (1H, d, J = 2.0 Hz), 8.98 (1H, dd, J = 4.1, 1.7 Hz). |
| 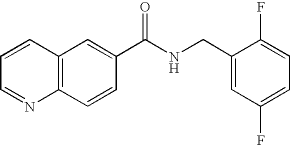 | 1H-NMR (DMSO-D6) δ: 4.57 (2H, d, J = 5.6 Hz), 7.14-7.30 (3H, m), 7.63 (1H, dd, J = 8.3, 4.1 Hz), 8.11 (1H, d, J = 8.8 Hz), 8.22 (1H, dd, J = 8.8, 2.0 Hz), 8.50 (1H, d, J = 6.8 Hz), 8.58 (1H, d, J = 2.0 Hz), 9.00 (1H, dd, J = 4.0, 1.6 Hz), 9.32 (1H, t, J = 5.6 Hz). |
| 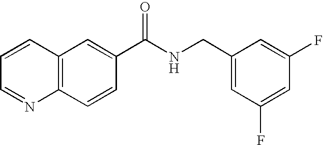 | 1H-NMR (CDCl3) δ: 4.69 (2H, d, J = 5.8 Hz), 6.71- 6.91 (4H, m), 7.48 (1H, dd, J = 8.2, 4.1 Hz), 8.07 (1H, dd, J = 8.7, 1.9 Hz), 8.16 (1H, d, J = 8.7 Hz), 8.23 (1H, d, J = 8.0 Hz), 8.34-8.36 (1H, m), 9.00 (1H, dd, J = 4.3, 1.7 Hz). |
| 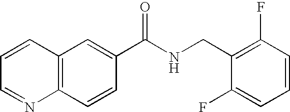 | 1H-NMR (CDCl3) δ: 4.82 (2H, d, J = 5.6 Hz), 6.68 (1H, br s), 6.90 6.97 (2H, m), 7.24-7.33 (1H, m), 7.46 (1H, dd, J = 8.3, 4.2 Hz), 8.04 (1H dd, J = 8.7, 1.9 Hz), 8.14 (1H, d, J = 8.7 Hz), 8.23 (1H, d, J = 8.2 Hz), 8.31 (1H, d, J = 1.7 Hz), 8.98 (1H, dd, J = 4.1, 1.7 Hz). |
| 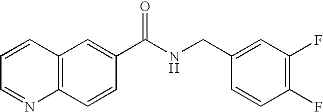 | 1H-NMR (CDCl3) δ: 4.67 (2H, d, J = 6.0 Hz), 6.65 (1H, br s), 7.11-7.27 (3H, m), 7.49 (1H, dd, J = 8.3, 4.2 Hz), 8.06 (1H, dd, J = 8.7, 1.9 Hz), 8.17 (1H, d, J = 8.9 Hz), 8.25 (1H, d, J = 7.7 Hz), 8.34 (1H, d, J = 1.7 Hz), 9.00 (1H, dd, J = 4.1, 1.7 Hz). |
| 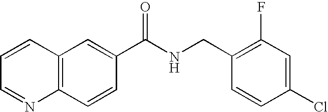 | 1H-NMR (CDCl3) δ: 4.72 (2H, d, J = 5.9 Hz), 6.73 (1H, br s), 7.11-7.15 (2H, m), 7.40-7.49 (2H, m), 8.05 (1H, d, J = 8.8 Hz), 8.15 (1H, d, J = 8.8 Hz), 8.23 (1H, d, J = 8.3 Hz), 8.31 (1H, s), 8.98-9.00 (1H, m). |
| 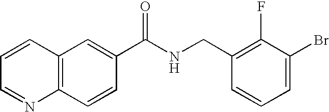 | 1H-NMR (CDCl3) δ: 4.76 (2H, d, J = 6.1 Hz), 6.88 (1H, s), 7.02 (1H, t, J = 7.8 Hz), 7.40-7.51 (3H, m), 8.06 (1H, dd, J = 8.8, 1.7 Hz), 8.14 (1H, d, J = 8.8 Hz), 8.22 (1H, d, J = 8.3 Hz), 8.32 (1H, d, J = 1.7 Hz), 8.98 (1H, dd, J = 4.1, 1.5 Hz). |
| 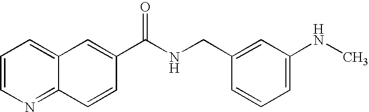 | 1H-NMR (DMSO-D6) δ: 2.65 (3H, d, J = 4.9 Hz), 4.44 (2H, d, J = 5.9 Hz), 5.62 (1H, d, J = 4.9 Hz), 6.41 (1H, d, J = 8.3 Hz), 6.53-6.56 (2H, m), 7.05 (1H, t, J = 7.9 Hz), 7.61 (1H, dd, J = 8.3, 4.1 Hz), 8.09 (1H, d, J = 8.8 Hz), 8.22 (1H, dd, J = 8.8, 2.0 Hz), 8.48 (1H, d, J = 7.6 Hz), 8.56 (1H, d, J = 1.5 Hz), 8.99 (1H, dd, J = 4.1, 1.7 Hz), 9.20 (1H, t, J = 5.6 Hz). |
| 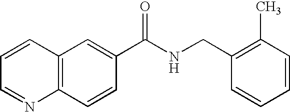 | 1H-NMR (CDCl3) δ: 2.41 (3H, s), 4.71 (2H, d, J = 5.4 Hz), 6.43 (1H, br s), 7.20-7.26 (3H, m), 7.34 (1H, d, J = 6.3 Hz), 7.46 (1H, dd, J = 8.3, 4.1 Hz), 8.05 (1H, dd, J = 8.8, 2.0 Hz), 8.14 (1H, d, J = 8.8 Hz), 8.22 (1H, d, J = 7.3 Hz), 8.31 (1H, d, J = 1.7 Hz), 8.98 (1H, dd, J = 4.1, 1.7 Hz). |
| 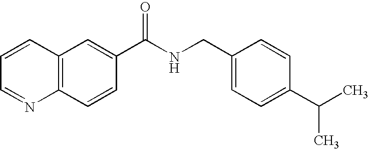 | 1H-NMR (CDCl3) δ: 1.26 (6H, d, J = 7.1 Hz), 2.89- 2.96 (1H, m), 4.68 (2H, d, J = 5.4 Hz), 6.51 (1H, br s), 7.24-7.35 (4H, m), 7.47 (1H, dd, J = 8.3, 4.1 Hz), 8.06 (1H, dd, J = 8.8, 2.0 Hz), 8.15 (1H, d, J = 8.8 Hz), (1H, d, J = 7.3 Hz), 8.33 (1H, d, J = 1.7 Hz), 8.99 (1H, dd, J = 4.1, 1.7 Hz). |
| 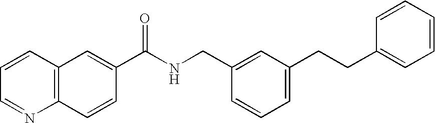 | 1H-NMR (CDCl3) δ: 2.93 (4H, s), 4.68 (2H, d, J = 4.9 Hz), 6.49 (1H, br s), 7.12-7.33 (9H, m), 7.46-7.51 (1H, m), 8.04-8.07 (1H, m), 8.17 (1H, d, J = 8.5 Hz), 8.25 (1H, d, J = 7.6 Hz), 8.32-8.35 (1H, m), 8.99- 9.02 (1H, m). |
| 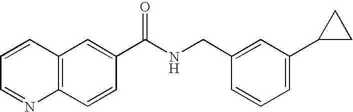 | 1H-NMR (CDCl3) δ: 0.69-0.73 (2H, m), 0.95-1.00 (2H, m), 1.86-1.94 (1H, m), 4.67 (2H, d, J = 5.6 Hz), 6.53 (1H, s), 7.01 (1H, d, J = 7.6 Hz), 7.12 (1H, s), 7.18 (1H, d, J = 7.8 Hz), 7.27 (1H, t, J = 7.2 Hz), 7.47 (1H, dd, J = 8.3, 4.1 Hz), 8.06 (1H, dd, J = 8.8, 2.2 Hz), 8.15 (1H, d, J = 8.8 Hz), 8.23 (1H, d, J = 7.6 Hz), 8.33 (1H, d, J = 2.0 Hz), 8.99 (1H, dd, J = 4.1, 1.7 Hz). |
| 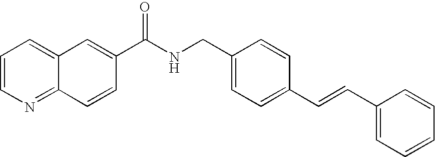 | 1H-NMR (DMSO-D6) δ: 4.56 (2H, d, J = 5.8 Hz), 7.23-7.30 (3H, m), 7.35-7.42 (4H, m), 7.57-7.64 (5H, m), 8.11 (1H, d, J = 8.7 Hz), 8.23 (1H, d, J = 8.5 Hz), 8.49 (1H, d, J = 7.7 Hz), 8.58 (1H, s), 9.00 (1H, d, J = 2.7 Hz), 9.32 (1H, t, J = 5.3 Hz). |
| 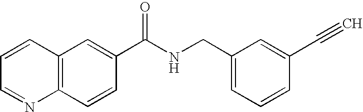 | 1H-NMR (CDCl3) δ: 3.09 (1H, s), 4.70 (2H, d, J = 5.6 Hz), 6.59 (1H, s), 7.34 (1H, t, J = 7.6 Hz), 7.40 (1H, d, J = 7.8 Hz), 7.43-7.49 (2H, m), 7.53 (1H, s), 8.07 (1H, dd, J = 8.8, 2.0 Hz), 8.17 (1H, d, J = 8.8 Hz), 8.24 (1H, dd, J = 8.4, 1.1 Hz), 8.34 (1H, d, J = 2.0 Hz), 9.00 (1H, dd, J = 4.1, 1.7 Hz). |
| 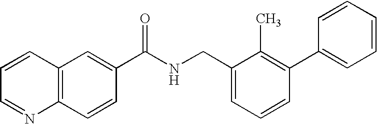 | 1H-NMR (DMSO-D6) δ: 2.23 (3H, s), 4.60 (2H, d, J = 5.4 Hz), 7.13 (1H, d, J = 7.3 Hz), 7.25 (1H, t, J = 7.6 Hz), 7.31-7.47 (6H, m), 7.62 (1H, dd, J = 8.0, 3.7 Hz), 8.10 (1H, d, J = 8.8 Hz), 8.25 (1H, d, J = 8.8 Hz), 8.50 (1H, d, J = 8.0 Hz), 8.60 (1H, s), 8.99-9.00 (1H, m), 9.20 (1H, s). |
| 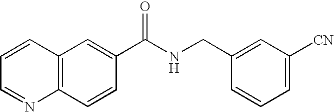 | 1H-NMR (DMSO-D6) δ: 4.60 (2H, d, J = 5.9 Hz), 7.58 (1H, t, J = 7.7 Hz), 7.62 (1H, dd, J = 8.3, 4.1 Hz), 7.71-7.76 (2H, m), 7.82 (1H, s), 8.10 (1H, d, J = 9.0 Hz), 8.22 (1H, dd, J = 8.8, 2.0 Hz), 8.49 (1H, dd, J = 8.4, 1.1 Hz), 8.57 (1H, d, J = 1.7 Hz), 9.00 (1H, dd, J = 4.1, 1.7 Hz), 9.37 (1H, t, J = 5.9 Hz). |
| 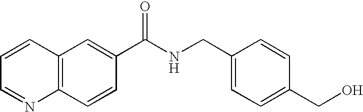 | 1H-NMR (CDCl3) δ: 4.02 (1H, t, J = 5.6 Hz), 4.62- 4.70 (4H, m), 7.37-7.41 (4H, m), 7.48 (1H, dd, J = 8.3, 4.5 Hz), 8.01 (1H, s), 8.13 (1H, d, J = 8.9 Hz), 8.20 (1H, dd, J = 8.8, 1.8 Hz), 8.27 (1H, d, J = 7.7 Hz), 8.44 (1H, d, J = 1.9 Hz), 8.98 (1H, dd, J = 4.0, 1.6 Hz). |
| 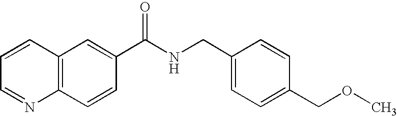 | 1H-NMR (CDCl3) δ: 3.40 (3H, s), 4.46 (2H, s), 4.71 (2H, d, J = 5.8 Hz), 6.58 (1H, br s), 7.34-7.40 (4H, m), 7.47 (1H, dd, J = 8.3, 4.2 Hz), 8.06 (1H, dd, J = 8.7, 1.9 Hz), 8.15 (1H, d, J = 8.7 Hz), 8.23 (1H, d, J = 8.5 Hz), 8.33 (1H, d, J = 1.7 Hz), 8.99 (1H, dd, J = 4.3, 1.7 Hz). |
| 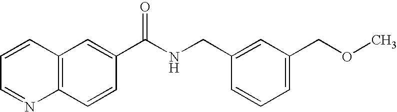 | 1H-NMR (CDCl3) δ: 3.41 (3H, s), 4.46 (2H, s), 4.71 (2H, d, J = 5.6 Hz), 6.64 (1H, br s), 7.28-7.39 (4H, m), 7.47 (1H, dd, J = 8.3, 4.2 Hz), 8.05-8.09 (1H, m), 8.15 (1H, d, J = 8.9 Hz), 8.23 (1H, d, J = 8.2 Hz), 8.32-8.34 (1H, m), 8.96-9.00 (1H, m). |
| 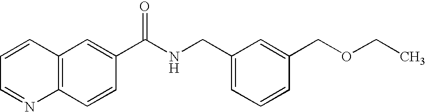 | 1H-NMR (CDCl3) δ: 1.25 (3H, t, J = 7.0 Hz), 3.57 (2H, q, J = 7.0 Hz), 4.51 (2H, s), 4.71 (2H, d, J = 5.6 Hz), 6.61 (1H, br s), 7.29-7.39 (4H, m), 7.47 (1H, dd, J = 8.3, 4.1 Hz), 8.07 (1H, dd, J = 8.8, 1.7 Hz), 8.15 (1H, d, J = 8.8 Hz), 8.23 (1H, d, J = 8.3 Hz), 8.32- 8.35 (1H, m), 8.98-8.99 (1H, m). |
| 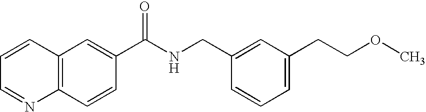 | 1H-NMR (CDCl3) δ: 2.90 (2H, t, J = 6.7 Hz), 3.35 (3H, s), 3.62 (2H, t, J = 6.7 Hz), 4.70 (2H, d, J = 5.1 Hz), 6.58 (1H, br s), 7.18-7.33 (4H, m), 7.47 (1H, dd, J = 8.2, 4.0 Hz), 8.05-8.09 (1H, m), 8.16 (1H, d, J = 8.5 Hz), 8.24 (1H, J = 8.3 Hz), 8.33-8.35 (1H, m), 8.98-9.01 (1H, m). |
| 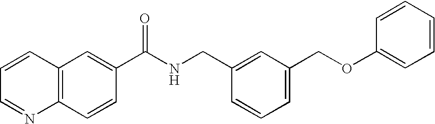 | 1H-NMR (CDCl3) δ: 4.74 (2H, d, J = 5.8 Hz), 5.08 (2H, s), 6.55 (1H, br s) 6.95-6.99 (3H, m), 7.26-7.31 (2H, m), 7.36-7.50 (5H, m), 8.06 (1H, dd, J = 8.9, 1.9 Hz), 8.16 (1H, d, J = 8.9 Hz), 8.24 (1H, d, J = 7.7 Hz), 8.33 (1H, d, J = 1.9 Hz), 9.00 (1H, dd, J = 4.2, 1.6 Hz). |
| 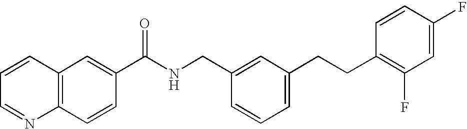 | 1H-NMR (CDCl3) δ: 4.73 (2H, d, J = 5.6 Hz), 5.09 (2H, s), 6.63 (1H, br s), 6.73-6.99 (3H, m), 7.37-7.39 (3H, m), 7.45-7.49 (2H, m), 8.04-8.08 (1H, d, J = 8.16 (1H, d, J = 8.8 Hz), 8.23 (1H, d, J = 8.3 Hz), 8.33 (1H, d, J = 2.0 Hz), 8.99 (1H, dd, J = 4.3, 1.8 Hz). |
| 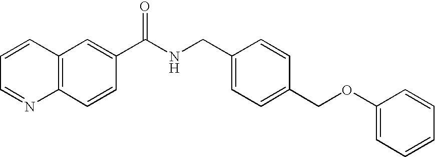 | 1H-NMR (CDCl3) δ: 4.71 (2H, d, J = 5.6 Hz), 5.06 (2H, s), 6.64 (1H, br s), 6.94-6.99 (3H, m), 7.26-7.30 (2H, m), 7.40-7.48 (5H, m), 8.06 (1H, dd, J = 8.8, 1.7 Hz), 8.14 (1H, d, J = 8.8 Hz), 8.22 (1H, d, J = 7.6 Hz), 8.32 (1H, d, J = 1.7 Hz), 8.98 (1H, dd, J = 4.1, 1.7 Hz). |
| 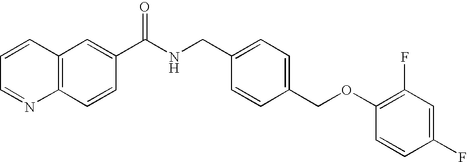 | 1H-NMR (CDCl3) δ: 4.72 (2H, d, J = 5.6 Hz), 5.03- 5.10 (2H, m), 6.59 (1H, br s), 6.72-6.99 (3H, m), 7.41-7.45 (4H, m), 7.48 (1H, dd, J = 8.3, 4.1 Hz), 8.07 (1H, dd, J = 8.8, 2.0 Hz), 8.16 (1H, d, J = 8.8 Hz), 8.23 (1H, d, J = 8.0 Hz), 8.33 (1H, d, J = 2.0 Hz), 8.98-9.01 (1H, m). |
| 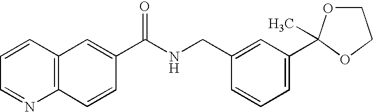 | 1H-NMR (CDCl3) δ: 1.66 (3H, s), 3.77-3.82 (2H, m), 4.02-4.08 (2H, m), 4.73 (2H, d, J = 5.6 Hz), 6.61 (1H, s), 7.34-7.37 (2H, m), 7.44-7.51 (3H, m), 8.07 (1H, dd, J = 8.8, 2.0 Hz), 8.15 (1H, d, J = 8.8 Hz), 8.23 (1H, d, J = 7.6 Hz), 8.34 (1H, d, J = 1.7 Hz), 8.99 (1H, dd, J = 4.1, 1.7 Hz). |
| 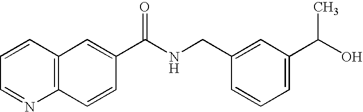 | 1H-NMR (DMSO-D6) δ: 1.30 (3H, d, J = 6.3 Hz), 4.52 (2H, s), 4.67-4.73 (1H, m), 5.12 (1H, br s), 7.30-7.32 (4H, m), 7.61 (1H, dd, J = 8.3, 4.4 Hz), 8.09 (1H, d, J = 8.8 Hz), 8.20-8.23 (1H, m), 8.48 (1H, dd, J = 8.0, 1.2 Hz), 8.55 (1H, d, J = 2.0 Hz), 8.99 (1H, dd, J = 4.1, 1.7 Hz), 9.27 (1H, br s). |
| 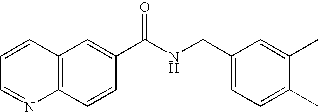 | 1H-NMR (CDCl3) δ: 2.26 (3H, s), 2.27 (3H, s), 4.64 (2H, d, J = 5.6 Hz), 6.57 (1H, br s), 7.13-7.17 (3H, m), 7.46 (1H, d, J = 8.2, 4.3 Hz), 8.06 (1H, d, J = 8.7, 1.9 Hz), 8.14 (1H, d, J = 8.7 Hz), 8.21 (1H, dd, J = 8.5, 1.0 Hz), 8.32 (1H, d, J = 1.9 Hz), 8.98 (1H, dd, J = 4.3, 1.7 Hz). |
| 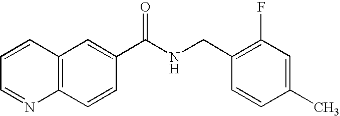 | 1H-NMR (CDCl3) δ: 2.35 (3H, s), 4.72 (2H, d, J = 5.8 Hz), 6.60 (1H, br s), 6.91-6.97 (2H, m), 7.29-7.36 (1H, m), 7.47 (1H, dd, J = 8.2, 4.1 Hz), 8.05 (1H, dd, J = 8.7, 1.9 Hz), 8.15 (1H, d, J = 8.7 Hz), 8.24 (1H, d, J = 8.2 Hz), 8.31 (1H, d, J = 1.9 Hz), 8.99 (1H, dd, J = 4.1, 1.7 Hz). |
| 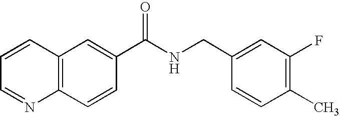 | 1H-NMR (CDCl3) δ: 2.27 (3H, d, J = 5.1 Hz), 4.67 (2H, d, J = 5.6 Hz), 6.55 (1H, br s), 7.01-7.08 (2H, m), 7.16-7.21 (1H, m), 7.48 (1H, dd, J = 8.3, 4.2 Hz), 8.06 (1H, dd, J = 8.8, 2.1 Hz), 8.17 (1H, d, J = 8.7 Hz), 8.25 (1H, d, J = 8.2 Hz), 8.34 (1H, d, J = 1.7 Hz), 9.00 (1H, dd, J = 4.1, 1.7 Hz). |
| 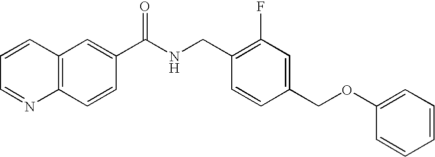 | 1H-NMR (CDCl3) δ: 4.76 (2H, d, J = 5.9 Hz), 5.06 (2H, s), 6.68 (1H, br s), 6.93-7.00 (3H, m), 7.18-7.32 (4H, m), 7.44-7.50 (2H, m), 8.05 (1H, dd, J = 8.8, 1.7 Hz), 8.15 (1H, d, J = 8.8 Hz), 8.23 (1H, d, J = 8.0 Hz), 8.31 (1H, d, J = 1.5 Hz), 8.99 (1H, d, J = 2.9 Hz). |
| 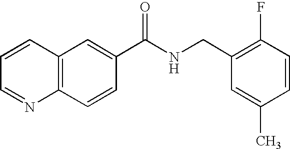 | 1H-NMR (CDCl3) δ: 2.32 (3H, s), 4.72 (2H, d, J = 5.6 Hz), 6.65 (1H, br s), 6.98 (1H, dd, J = 9.9, 8.4 Hz), 7.07-7.10 (1H, dd, 7.24-7.27 (1H, m), 7.47 (1H, dd, J = 8.3, 4.1 Hz), 8.06 (1H, dd, J = 8.8, 1.7 Hz), 8.16 (1H, d, J = 8.8 Hz), 8.25 (1H, d, J = 8.5 Hz), 8.32 (1H, s), 8.99 (1H, t, J = 2.1 Hz). |
| 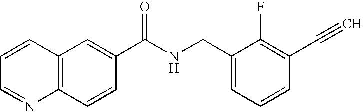 | 1H-NMR (CDCl3) δ: 3.32 (1H, s), 4.74 (2H, d, J = 5.6 Hz), 6.93 (1H, s), 7.09 (1H, t, J = 7.7 Hz), 7.40- 7.48 (3H, m), 8.06 (1H, dd, J = 8.8, 2.0 Hz), 8.13 (1H, d, J = 8.8 Hz), 8.20 (1H, d, J = 8.3 Hz), 8.31 (1H, d, J = 2.0 Hz), 8.97 (1H, dd, J = 4.3, 1.6 Hz). |
| 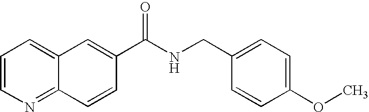 | 1H-NMR (CDCl3) δ: 3.81 (3H, s), 4.64 (2H, d, J = 5.6 Hz), 6.52 (1H, br s), 6.88-6.93 (2H, m), 7.31-7.35 (2H, m), 7.46 (1H, dd, J = 8.3, 4.4 Hz), 8.05 (1H, dd, J = 8.8, 2.0 Hz), 8.14 (1H, d, J = 8.8 Hz), 8.22 (1H, d, J = 8.3 Hz), 8.31 (1H, d, J = 2.0 Hz), 8.98 (1H, dd, J = 4.1, 7.7 Hz). |
| 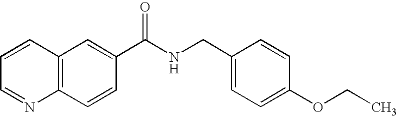 | 1H-NMR (CDCl3) δ: 1.42 (3H, t, J = 7.0 Hz), 4.04 (2H, q, J = 7.0 Hz), 4.64 (2H, d, J = 5.3 Hz), 6.49 (1H, br s), 6.88-6.92 (2H, m), 7.29-7.33 (2H, m), 7.47 (1H, dd, J = 8.3, 4.2 Hz), 8.05 (1H, dd, J = 8.7, 1.9 Hz), 8.15 (1H, d, J = 8.9 Hz), 8.23 (1H, d, J = 8.2 Hz), 8.32 (1H, d, J = 1.9 Hz), 8.99 (1H, dd, J = 4.1, 1.7 Hz). |
| 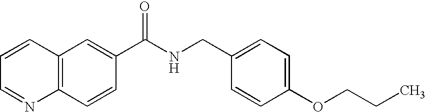 | 1H-NMR (CDCl3) δ: 1.04 (3H, t, J = 7.3 Hz), 7.77- 1.84 (2H, m), 3.92 (2H, t, J = 6.6 Hz), 4.64 (2H, d, J = 5.4 Hz), 6.48 (1H, br s), 6.90 (2H, d, J = 8.3 Hz), 7.31 (2H, d, J = 8.5 Hz), 7.47 (1H, dd, J = 8.2, 4.0 Hz), 8.05 (1H, d, J = 8.8 Hz), 8.15 (1H, d, J = 8.5 Hz), 8.23 (1H, d, J = 8.5 Hz), 8.32 (1H, s), 8.98 (1H, d, J = 2.4 Hz). |
| 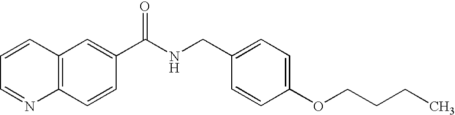 | 1H-NMR (CDCl3) δ: 0.98 (3H, t, J = 7.4 Hz), 1.44- 1.54 (2H, m), 1.73-1.80 (2H, m), 3.97 (2H, t, J = 6.5 Hz), 4.64 (2H, d, J = 5.1 Hz), 6.47 (1H, br s), 6.90 (2H, d, J = 8.3 Hz), 7.31 (2H, d, J = 8.3 Hz), 7.47 (1H, dd, J = 8.4, 4.3 Hz), 8.05 (1H, d, J = 9.0 Hz), 8.15 (1H, d, J = 9.0 Hz), 8.23 (1H, d, J = 8.0 Hz), 8.32 (1H, s), 8.98 (1H, d, J = 4.1 Hz). |
| 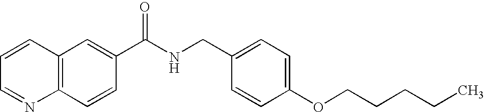 | 1H-NMR (CDCl3) δ: 0.93 (3H, t, J = 6.8 Hz), 1.35- 1.47 (4H, m), 1.76-1.82 (2H, m), 3.95 (2H, t, J = 6.5 Hz), 4.64 (2H, d, J = 5.1 Hz), 6.52 (1H, br s), 6.90 (2H, d, J = 8.0 Hz), 7.31 (2H, d, J = 8.5 Hz), 7.46 (1H, dd, J = 7.9, 4.0 Hz), 8.05 (1H, d, J = 9.0 Hz), 8.14 (1H, d, J = 8.8 Hz), 8.23 (1H, d, J = 8.3 Hz), 8.31 (1H, s), 8.98 (1H, d, J = 3.2 Hz). |
| 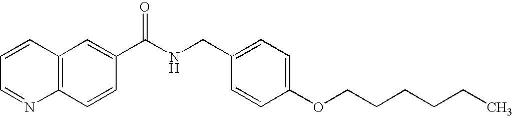 | 1H-NMR (CDCl3) δ: 0.88-0.93 (3H, m), 1.32-1.49 (4H, m), 1.59-1.64 (2H, m), 1.75-1.80 (2H, m), 3.95 (2H, t, J = 6.3 Hz), 4.63-4.65 (2H, m), 6.50 (1H, br s), 6.90 (2H, d, J = 8.0 Hz), 7.26-7.34 (2H, m), 7.44- 7.49 (1H, m), 8.05 (1H, d, J = 8.7 Hz), 8.15 (1H, d, J = 8.9 Hz), 8.23 (1H, d, J = 7.5 Hz), 8.32 (1H, s), 8.98 (1H, s). |
| 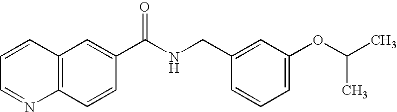 | 1H-NMR (CDCl3) δ: 1.34 (6H, d, J = 6.1 Hz), 4.54- 4.60 (1H, m), 4.68 (2H, d, J = 5.6 Hz), 6.55 (1H, br s), 6.84 (1H, dd, J = 8.4, 2.1 Hz), 6.92-6.96 (2H, m), 7.26-7.30 (1H, m), 7.47 (1H, dd, J = 8.3, 4.1 Hz), 8.07 (1H, dd, J = 8.8, 2.0 Hz), 8.16 (1H, d, J = 8.8 Hz), 8.24 (1H, d, J = 8.3 Hz), 8.33 (1H, d, J = 2.0 Hz), 8.99 (1H, dd, J = 4.1, 1.7 Hz). |
| 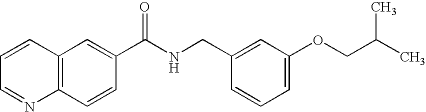 | 1H-NMR (CDCl3) δ: 1.02 (6H, d, J = 6.6 Hz), 2.04- 2.12 (1H, m), 3.73 (2H, d, J = 6.6 Hz), 4.68 (2H, d, J = 5.4 Hz), 6.53 (1H, br s), 6.86 (1H, dd, J = 8.2, 2.3 Hz), 6.94-6.98 (2H, m), 7.26-7.31 (1H, m), 7.48 (1H, dd, J = 8.3, 4.4 Hz), 8.07 (1H, dd, J = 8.8, 2.0 Hz), 8.16 (1H, d, J = 8.8 Hz), 8.25 (1H, d, J = 8.0 Hz), 8.33 (1H, d, J = 1.7 Hz), 8.99 (1H, dd, J = 4.1, 1.5 Hz). |
| 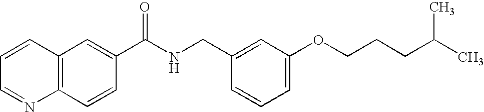 | 1H-NMR (CDCl3) δ: 0.91 (6H, d, J = 6.5 Hz), 1.30- 1.36 (2H, m), 1.57-1.63 (1H, dd, 1.74-1.82 (2H, m), 3.95 (2H, t, J = 6.6 Hz), 4.68 (2H, d, J = 5.6 Hz), 6.53 (1H, s), 6.85 (1H, dd, J = 8.2, 2.4 Hz), 6.94-6.97 (2H, m), 7.28 (1H, t, J = 8.5 Hz), 7.48 (1H, dd, J = 8.2, 4.1 Hz), 8.07 (1H, dd, J = 8.7, 1.9 Hz), 8.16 (1H, d, J = 8.9 Hz), 8.24 (1H, dd, J = 8.5, 1.0 Hz), 8.33 (1H, d, J = 1.9 Hz), 8.99 (1H, dd, J = 4.1, 1.7 Hz). |
| 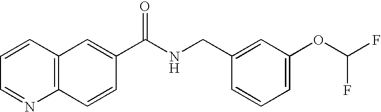 | 1H-NMR (CDCl3) δ: 4.72 (2H, d, J = 5.6 Hz), 6.52 (1H, t, J = 73.7 Hz), 6.68 (1H, br s), 1.07 (1H, d, J = 8.0 Hz), 7.15 (1H, s), 7.23-7.27 (1H, m), 7.35- 7.39 (1H, d), 1.48 (1H, dd, J = 8.3, 4.1 Hz), 8.07 (1H, dd, J = 8.9, 1.6 Hz), 8.16 (1H, d, J = 8.8 Hz), 8.23 (1H, d, J = 8.0 Hz), 8.33-8.35 (1H, m), 8.99 (1H, d, J = 2.9 Hz). |
| 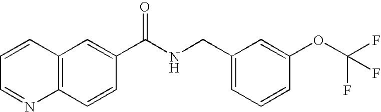 | 1H-NMR (CDCl3) δ: 4.74 (2H, d, J = 5.9 Hz), 6.72 (1H, br s), 7.15-7.27 (2H, m), 7.32-7.42 (2H, m), 7.48 (1H, dd, J = 8.3, 4.1 Hz), 8.07 (1H, dd, J = 8.8, 2.0 Hz), 8.16 (1H, d, J = 8.8 Hz), 8.24 (1H, d, J = 8.5 Hz), 8.34 (1H, d, J = 2.0 Hz), 8.99 (1H, dd, J = 4.4, 1.7 Hz). |
| 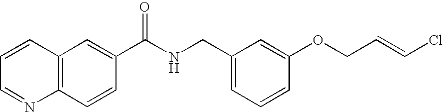 | 1H-NMR (CDCl3) δ: 4.53 (2H, d, J = 5.1 Hz), 4.69 (2H, d, J = 5.6 Hz), 6.13-6.19 (1H, m), 6.38 (1H, d, J = 13.4 Hz), 6.55 (1H, s), 6.84-6.86 (1H, m), 6.94- 6.95 (1H, m), 7.01 (1H, d, J = 7.8 Hz), 7.30 (1H, t, J = 7.9 Hz), 7.48 (1H, dd, J = 8.2, 4.0 Hz), 8.07 (1H, dd, J = 9.0, 2.0 Hz), 8.17 (1H, d, J = 8.5 Hz), 8.25 (1H, d, J = 8.0 Hz), 8.34 (1H, d, J = 1.7 Hz), 8.99- 9.00 (1H, m). |
| 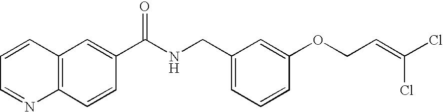 | 1H-NMR (DMSO-D6) δ: 4.53 (2H, d, J = 5.9 Hz), 4.67 (2H, d, J = 6.6 Hz), 6.46 (1H, t, J = 6.5 Hz), 6.86 (1H, dd, J = 7.9, 2.3 Hz), 6.95-6.99 (2H, m), 7.28 (1H, t, J = 7.9 Hz), 7.62 (1H, dd, J = 8.3, 4.1 Hz), 8.09 (1H, d, J = 8.8 Hz), 8.22 (1H, dd, J = 8.8, 2.0 Hz), 8.47-8.49 (1H, m), 8.57 (1H, d, J = 2.0 Hz), 8.99 (1H, dd, J = 4.3, 1.8 Hz), 9.28 (1H, t, J = 5.9 Hz). |
| 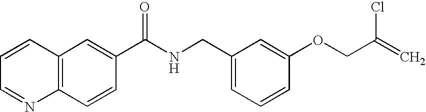 | 1H-NMR (DMSO-D6) δ: 4.52 (2H, d, J = 5.9 Hz), 4.70 (2H, s), 5.50 (1H, d, J = 1.7 Hz), 5.70 (1H, d, J = 1.2 Hz), 6.89 (1H, dd, J = 8.9, 2.1 Hz), 6.98-7.00 (2H, m), 7.27 (1H, t, J = 8.0 Hz), 7.62 (1H, dd, J = 8.3, 4.1 Hz), 8.09 (1H, d, J = 8.8 Hz), 8.22 (1H, dd, J = 8.8, 2.0 Hz), 8.49 (1H, d, J = 8.3 Hz), 8.56 (1H, d, J = 2.0 Hz), 8.99 (1H, dd, J = 4.1, 1.7 Hz), 9.27 (1H, t, J = 6.0 Hz). |
| 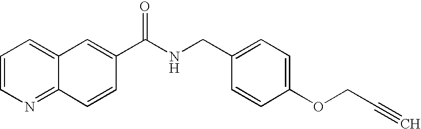 | 1H-NMR (CDCl3) δ: 2.52 (1H, t, J = 2.3 Hz), 4.64 (2H, d, J = 5.4 Hz), 4.69 (2H, d, J = 2.4 Hz), 6.62 (1H, br s), 6.95-7.00 (2H, m), 7.32-7.35 (2H, m), 7.45 (1H, dd, J = 8.3, 4.1 Hz), 8.05 (1H, dd, J = 8.8, 2.2 Hz), 8.13 (1H, d, J = 9.0 Hz), 8.21 (1H, d, J = 8.3 Hz), 8.31 (1H, d, J = 2.0 Hz), 8.97 (1H, dd, J = 4.1, 1.7 Hz). |
| 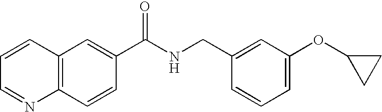 | 1H-NMR (CDCl3) δ: 0.77-0.79 (4H, m), 3.72-3.76 (1H, m), 4.70 (2H, d, J = 5.6 Hz), 6.56 (1H, s), 6.99- 7.07 (3H, m), 7.26-7.32 (1H, m), 7.48 (1H, dd, J = 8.2, 4.1 Hz), 8.07 (1H, d, J = 8.5 Hz), 8.16 (1H, d, J = 8.5 Hz), 8.24 (1H, d, J = 8.0 Hz), 8.33 (1H, s), 8.99 (1H, d, J = 2.9 Hz). |
| 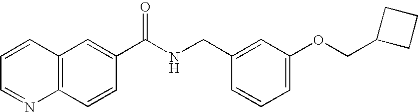 | 1H-NMR (CDCl3) δ: 1.84-2.00 (4H, m), 2.10-2.17 (2H, m), 2.73-2.80 (1H, m), 3.94 (2H, d, J = 6.6 Hz), 4.68 (2H, d, J = 5.6 Hz), 6.51 (1H, s), 6.86 (1H, dd, J = 8.4, 1.8 Hz), 6.94-6.97 (2H, m), 7.28 (1H, t, J = 8.8 Hz), 7.48 (1H, dd, J = 8.3, 4.4 Hz), 8.07 (1H, dd, J = 8.7, 1.8 Hz), 8.16 (1H, d, J = 9.0 Hz), 8.24 (1H, d, J = 8.5 Hz), 8.33 (1H, d, J = 1.7 Hz), 8.99 (1H, dd, J = 4.1, 1.7 Hz). |
| 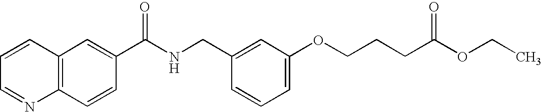 | 1H-NMR (CDCl3) δ: 7.24 (3H, t, J = 7.1 Hz), 2.07- 2.14 (2H, m), 2.51 (2H, t, J = 7.3 Hz), 4.02 (2H, t, J = 6.2 Hz), 4.13 (2H, q, J = 7.1 Hz), 4.68 (2H, d, J = 5.6 Hz), 6.54 (1H, s), 6.84 (1H, dd, J = 8.2, 2.1 Hz), 6.93 (1H, s), 6.97 (1H, d, J = 7.3 Hz), 7.28 (1H, t, J = 8.4 Hz), 7.48 (1H, dd, J = 8.4, 4.3 Hz), 8.07 (1H, dd, J = 8.8, 1.7 Hz), 8.16 (1H, d, J = 8.8 Hz), 8.24 (1H, d, J = 8.3 Hz), 8.34 (1H, d, J = 1.7 Hz), 8.99 (1H, dd, J = 4.1, 1.5 Hz). |
| 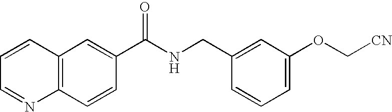 | 1H-NMR (CDCl3) δ: 4.72 (2H, d, J = 5.8 Hz), 4.79 (2H, s), 6.72 (1H, br s), 6.94 (1H, d, J = 8.0 Hz), 7.04 (1H, s), 7.12 (1H, d, J = 7.5 Hz), 7.36 (1H, dd, J = 8.0, 7.7 Hz), 7.48 (1H, dd, J = 8.2, 4.1 Hz), 8.08 (1H, d, J = 8.7 Hz), 8.16 (1H, d, J = 8.7 Hz), 8.24 (1H, d, J = 8.2 Hz), 8.35 (1H, s), 8.99 (1H, d, J = 2.9 Hz). |
| 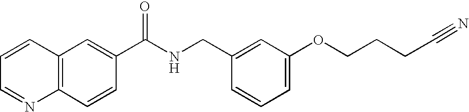 | 1H-NMR (CDCl3) δ: 2.11-2.17 (2H, m), 2.59 (2H, t, J = 7.0 Hz), 4.09 (2H, t, J = 5.7 Hz), 4.69 (2H, d, J = 5.8 Hz), 6.61 (1H, s), 6.85 (1H, dd, J = 8.0, 1.9 Hz), 6.95 (1H, s), 7.01 (1H, d, J = 7.5 Hz), 7.25-7.32 (1H, m), 7.48 (1H, dd, J = 8.2, 4.1 Hz), 8.08 (1H, dd, J = 8.7, 1.9 Hz), 8.16 (1H, d, J = 8.7 Hz), 8.24 (1H, d, J = 8.2 Hz), 8.34 (1H, d, J = 1.7 Hz), 8.99 (1H, dd, J = 4.1, 1.7 Hz). |
| 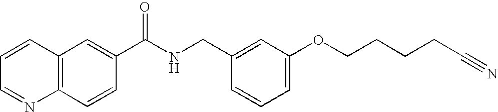 | 1H-NMR (CDCl3) δ: 1.86-1.96 (4H, m), 2.44 (2H, t, J = 6.5 Hz), 4.01 (2H, t, J = 5.2 Hz), 4.68 (2H, d, J = 5.3 Hz), 6.62 (1H, br s), 6.84 (1H, d, J = 7.5 Hz), 6.93 (1H, s), 6.99 (1H, d, J = 7.5 Hz), 7.26-7.31 (1H, m), 7.48 (1H, dd, J = 7.8, 4.0 Hz), 8.07 (1H, d, J = 8.2 Hz), 8.16 (1H, d, J = 8.9 Hz), 8.24 (1H, d, J = 8.2 Hz), 8.34 (1H, s), 8.99 (1H, s). |
| 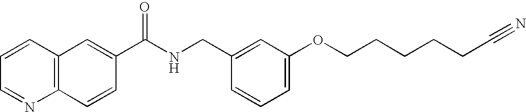 | 1H-NMR (CDCl3) δ: 1.62-1.86 (6H, m), 2.38 (2H, t, J = 7.0 Hz), 3.99 (2H, t, J = 6.1 Hz), 4.69 (2H, d, J = 5.6 Hz), 6.60 (1H, br s), 6.82-6.86 (1H, m), 6.93- 6.99 (2H, m), 7.27-7.31 (1H, m), 7.48 (1H, dd, J = 8.3, 4.1 Hz), 8.07 (1H, dd, J = 8.8, 2.0 Hz), 8.16 (1H, d, J = 8.8 Hz), 8.24 (1H, d, J = 8.0 Hz), 8.34 (1H, d, J = 1.5 Hz), 8.99 (1H, t, J = 2.1 Hz). |
| 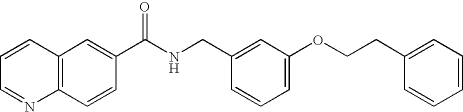 | 1H-NMR (CDCl3) δ: 3.10 (2H, t, J = 7.1 Hz), 4.18 (2H, t, J = 7.0 Hz), 4.67 (2H, d, J = 5.6 Hz), 6.54 (1H, br s), 6.85 (1H, dd, J = 8.1, 2.3 Hz), 6.93-6.98 (2H, m), 7.23-7.32 (6H, m), 7.47 (1H, dd, J = 8.3, 4.2 Hz), 8.06 (1H, dd, J = 8.7, 1.9 Hz), 8.16 (1H, d, J = 8.9 Hz), 8.23 (1H, d, J = 8.5 Hz), 8.32 (1H, d, J = 7.7 Hz), 8.99 (1H, dd, J = 4.3, 1.7 Hz). |
| 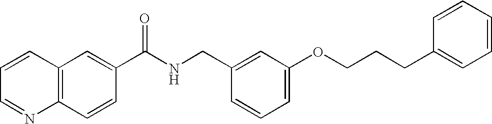 | 1H-NMR (CDCl3) δ: 2.07-2.14 (2H, m), 2.81 (2H, t, J = 7.6 Hz), 3.97 (2H, t, J = 6.2 Hz), 4.68 (2H, d, J = 5.4 Hz), 6.59 (1H, br s), 6.84-6.86 (1H, m), 6.93- 6.98 (2H, m), 7.16-7.30 (6H, m), 7.47 (1H, dd, J = 8.2, 4.3 Hz), 8.07 (1H, d, J = 8.8 Hz), 8.16 (1H, d, J = 8.5 Hz), 8.24 (1H, d, J = 7.8 Hz), 8.33 (1H, s), 8.98 (1H, d, J = 2.7 Hz). |
| 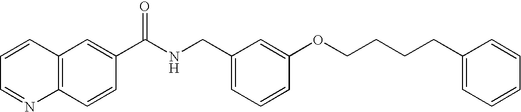 | 1H-NMR (CDCl3) δ: 1.79-1.83 (4H, m), 2.68 (2H, t, J = 7.1 Hz), 3.98 (2H, t, J = 6.0 Hz), 4.68 (2H, d, J = 5.6 Hz), 6.52 (1H, s), 6.84 (1H, dd, J = 7.9, 2.1 Hz), 6.92-6.92 (1H, m), 6.96 (1H, d, J = 7.6 Hz), 7.16-7.20 (3H, m), 7.26-7.30 (3H, m), 7.47 (1H, dd, J = 8.3, 4.4 Hz), 8.06 (1H, dd, J = 8.8, 2.2 Hz), 8.16 (1H, d, J = 8.8 Hz), 8.22-8.24 (1H, m), 8.33 (1H, d, J = 1.7 Hz), 8.99 (1H, dd, J = 4.4, 1.7 Hz). |
| 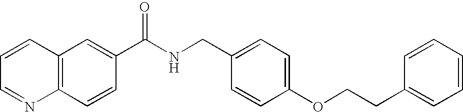 | 1H-NMR (CDCl3) δ: 3.10 (2H, t, J = 7.1 Hz), 4.18 (2H, t, J = 7.1 Hz), 4.63 (2H, d, J = 5.3 Hz), 6.51 (1H, br s), 6.90 (2H, d, J = 8.7 Hz), 7.22-7.34 (7H, m), 7.46 (1H, dd, J = 8.3, 4.2 Hz), 8.05 (1H, dd, J = 8.9, 1.9 Hz), 8.14 (1H, d, J = 8.7 Hz), 8.22 (1H, d, J = 8.5 Hz), 8.31 (1H, d, J = 1.7 Hz), 8.98 (1H, dd, J = 4.1, 1.7 Hz). |
| 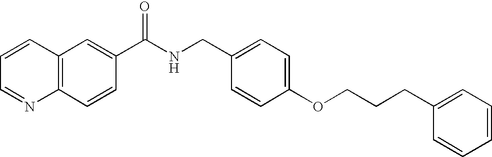 | 1H-NMR (CDCl3) δ: 2.07-2.14 (2H, m), 2.81 (2H, t, J = 7.5 Hz), 3.96 (2H, t, J = 6.3 Hz), 4.64 (2H, d, J = 5.3 Hz), 6.49 (1H, br s), 6.90 (2H, d, J = 8.5 Hz), 7.18-7.22 (3H, m), 7.26-7.33 (4H, m), 7.47 (1H, dd, J = 8.5, 4.1 Hz), 8.05 (1H, dd, J = 8.8, 1.8 Hz), 8.15 (1H, d, J = 8.7 Hz), 8.23 (1H, d, J = 8.2 Hz), 8.34 (1H, d, J = 16.2 Hz), 8.99 (1H, d, J = 2.9 Hz). |
| 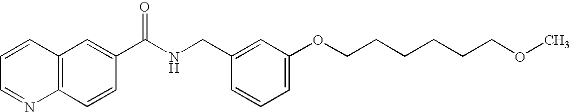 | 1H-NMR (CDCl3) δ: 1.34-1.62 (6H, m), 1.76-1.82 (2H, m), 3.32 (3H, s), 3.37 (2H, t, J = 6.5 Hz), 3.96 (2H, t, J = 6.5 Hz), 4.68 (2H, d, J = 5.6 Hz), 6.56 (1H, br s), 6.83-6.87 (1H, m), 6.92-6.99 (2H, m), 7.28-7.31 (1H, m), 7.47 (1H, dd, J = 8.3, 4.1 Hz), 8.07 (1H, dd, J = 8.8, 2.0 Hz), 8.16 (1H, d, J = 8.8 Hz), 8.24 (1H, d, J = 8.3 Hz), 8.33 (1H, d, J = 2.0 Hz), 8.99 (1H, dd, J = 4.3, 7.8 Hz). |
| 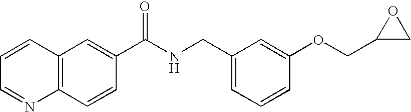 | 1H-NMR (DMSO-D6) δ: 2.70 (1H, dd, J = 5.1, 2.7 Hz), 2.83 (1H, t, J = 4.8 Hz), 3.31-3.34 (1H, m), 3.82 (1H, dd, J = 11.2, 6.6 Hz), 4.31 (1H, dd, J = 11.3, 2.6 Hz), 4.52 (2H, d, J = 5.9 Hz), 6.86 (1H, dd, J = 8.0, 1.7 Hz), 6.96-6.97 (2H, m), 7.26 (1H, t, J = 8.0 Hz), 7.62 (1H, dd, J = 8.3, 4.1 Hz), 8.09 (1H, d, J = 8.8 Hz), 8.22 (1H, dd, J = 8.9, 1.8 Hz), 8.49 (1H, d, J = 8.3 Hz), 8.56 (1H, d, J = 1.7 Hz), 8.99 (1H, dd, J = 4.0, 1.6 Hz), 9.27 (1H, t, J = 5.7 Hz). |
| 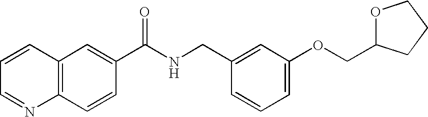 | 1H-NMR (CDCl3) δ: 1.71-1.80 (1H, m), 1.93-1.99 (2H, m), 2.03-2.11 (1H, m), 3.80-3.85 (1H, m), 3.90- 3.98 (3H, m), 4.24-4.30 (1H, m), 4.68 (2H, d, J = 5.6 Hz), 6.56 (1H, s), 6.87-6.89 (1H, m), 6.97-6.98 (2H, m), 7.26-7.30 (1H, m), 7.48 (1H, dd, J = 8.3, 4.4 Hz), 8.07 (1H, dd, J = 8.8, 2.0 Hz), 8.16 (1H, d, J = 8.8 Hz), 8.25 (1H, d, J = 8.3 Hz), 8.33 (1H, d, J = 1.7 Hz), 8.99 (1H, dd, J = 4.1, 1.7 Hz). |
| 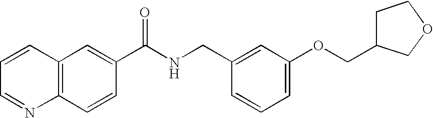 | 1H-NMR (CDCl3) δ: 1.69-1.77 (1H, m), 2.06-2.15 (1H, m), 2.68-2.78 (1H, m), 3.70 (1H, dd, J = 8.9, 5.2 Hz), 3.77 (1H, dd, J = 15.1, 8.0 Hz), 3.85-3.95 (4H, m), 4.68 (2H, d, J = 5.9 Hz), 6.65 (1H, s), 6.81-7.00 (3H, m), 7.26-7.30 (1H, m), 7.45-7.48 (1H, m), 8.07 (1H, dd, J = 8.8, 2.0 Hz), 8.15 (1H, d, J = 8.8 Hz), 8.23 (1H, dd, J = 8.4, 1.1 Hz), 8.34 (1H, d, J = 1.7 Hz), 8.99 (1H, dd, J = 4.1, 1.7 Hz). |
| 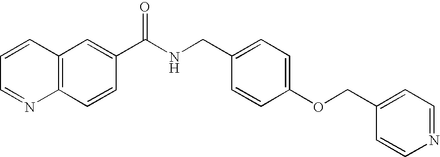 | 1H-NMR (CDCl3) δ: 4.61 (2H, d, J = 5.6 Hz), 5.05 (2H, s), 6.88-6.91 (2H, m), 7.29-7.36 (4H, m), 7.41 (1H, dd, J = 8.3, 4.1 Hz), 8.07 (1H, d, J = 8.8 Hz), 8.13-8.17 (2H, m), 8.38-8.39 (1H, m), 8.49-8.58 (3H, m), 8.92 (1H, dd, J = 4.0, 1.3 Hz). |
| 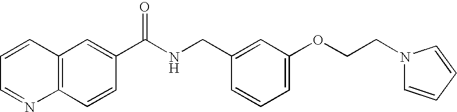 | 1H-NMR (CDCl3) δ: 4.20-4.23 (2H, m), 4.26-4.29 (2H, m), 4.66 (2H, d, J = 5.6 Hz), 6.15 (2H, t, J = 2.1 Hz), 6.54 (1H, s), 6.75 (2H, t, J = 2.2 Hz), 6.81 (1H, dd, J = 8.0, 2.0 Hz), 6.88-6.88 (1H, m), 6.98 (1H, d, J = 7.6 Hz), 7.27 (1H, t, J = 7.8 Hz), 7.47 (1H, dd, J = 8.3, 4.1 Hz), 8.06 (1H, dd, J = 8.8, 2.0 Hz), 8.16 (1H, d, J = 8.8 Hz), 8.24 (1H, d, J = 8.3 Hz), 8.33 (1H, d, J = 2.0 Hz), 8.99 (1H, dd, J = 4.3, 1.6 Hz). |
| 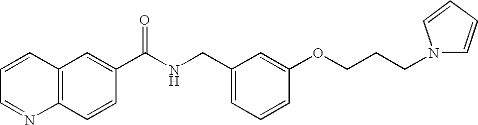 | 1H-NMR (CDCl3) δ: 2.17-2.23 (2H, m), 3.89 (2H, t, J = 5.9 Hz), 4.11 (2H, t, J = 6.8 Hz), 4.68 (2H, d, J = 5.8 Hz), 6.12 (2H, t, J = 2.1 Hz), 6.59 (1H, s), 6.64 (2H, t, J = 2.1 Hz), 6.83 (1H, dd, J = 8.2, 2.2 Hz), 6.91-6.92 (1H, m), 6.98 (1H, d, J = 7.5 Hz), 7.26- 7.30 (1H, m), 7.47 (1H, dd, J = 8.3, 4.2 Hz), 8.07 (1H, dd, J = 8.7, 1.9 Hz), 8.16 (1H, d, J = 8.7 Hz), 8.23 (1H, dd, J = 8.5, 1.2 Hz), 8.33 (1H, d, J = 1.9 Hz), 8.99 (1H, dd, J = 4.2, 1.8 Hz). |
| 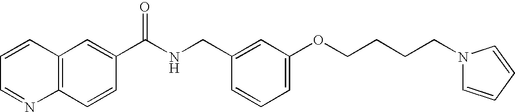 | 1H-NMR (CDCl3) δ: 1.73-1.79 (2H, m), 1.92-1.99 (2H, m), 3.92-3.97 (4H, m), 4.68 (2H, d, J = 5.6 Hz), 6.13 (2H, t, J = 2.1 Hz), 6.56 (1H, s), 6.66 (2H, t, J = 2.1 Hz), 6.82 (1H, dd, J = 7.9, 2.1 Hz), 6.90-6.91 (1H, m), 6.97 (1H, d, J = 7.6 Hz), 7.28 (1H, t, J = 7.6 Hz), 7.47 (1H, dd, J = 8.3, 4.1 Hz), 8.06 (1H, dd, J = 8.8, 2.0 Hz), 8.16 (1H, d, J = 8.8 Hz), 8.23 (1H, d, J = 8.3 Hz), 8.33 (1H, d, J = 2.0 Hz), 8.99 (1H, dd, J = 4.4, 1.7 Hz). |
| 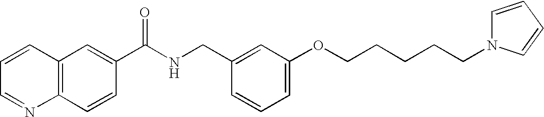 | 1H-NMR (CDCl3) δ: 1.42-1.50 (2H, m), 1.76-1.87 (4H, m), 3.89 (2H, t, J = 7.1 Hz), 3.94 (2H, t, J = 6.3 Hz), 4.68 (2H, d, J = 5.6 Hz), 6.13 (2H, t, J = 2.1 Hz), 6.55 (1H, s), 6.64 (2H, t, J = 2.1 Hz), 6.83 (1H, dd, J = 7.9, 2.1 Hz), 6.91-6.92 (1H, m), 6.96 (1H, d, J = 7.6 Hz), 7.25-7.30 (1H, m), 7.47 (1H, dd, J = 8.3, 4.1 Hz), 8.06 (1H, dd, J = 8.8, 2.0 Hz), 8.16 (1H, d, J = 8.8 Hz), 8.22-8.24 (1H, m), 8.33 (1H, d, J = 1.7 Hz), 8.99 (1H, dd, J = 4.1, 1.7 Hz). |
| 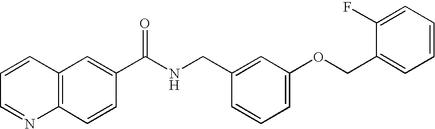 | 1H-NMR (CDCl3) δ: 4.70 (2H, d, J = 5.6 Hz), 5.14 (2H, s), 6.55 (1H, br s), 6.95 (1H, dd, J = 8.2, 2.2 Hz), 7.00-7.09 (3H, m), 7.15 (1H, dd, J = 7.5, 6.8 Hz), 7.26-7.33 (2H, m), 7.46-7.52 (2H, m), 8.06 (1H, dd, J = 8.9, 1.9 Hz), 8.16 (1H, d, J = 8.7 Hz), 8.24 (1H, d, J = 8.2 Hz), 8.33 (1H, d, J = 1.9 Hz), 8.99 (1H, dd, J = 4.3, 1.7 Hz). |
| 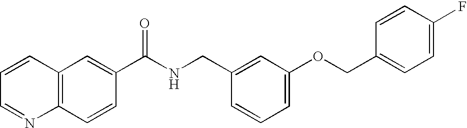 | 1H-NMR (CDCl3) δ: 4.69 (2H, d, J = 5.6 Hz), 5.03 (2H, s), 6.57 (1H, br s), 6.90-6.93 (1H, m), 6.99-7.06 (4H, m), 7.28-7.32 (1H, m), 7.37-7.41 (2H, m), 7.48 (1H, dd, J = 8.3, 4.2 Hz), 8.05 (1H, dd, J = 8.7, 1.9 Hz), 8.16 (1H, d, J = 8.9 Hz), 8.24 (1H, d, J = 8.2 Hz), 8.32 (1H, d, J = 1.7 Hz), 8.99 (1H, dd, J = 4.3, 1.7 Hz). |
| 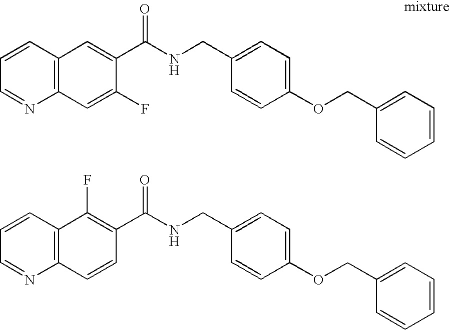 | 1H-NMR (DMSO-D6) δ: 4.22 (0.2H, d, J = 6.3 Hz), 4.45 (1.8H, d, J = 5.8 Hz), 5.09 (0.2H, s), 5.10 (1.8H, s), 6.99-7.46 (9.0H, m), 7.58 (0.9H, dd, J = 8.3, 4.2 Hz), 7.71 (0.1H, dd, J = 8.6, 4.0 Hz), 7.84 (0.9H, d, J = 11.8 Hz), 7.92-7.93 (0.2H, m), 8.32 (0.9H, d, J = 7.7 Hz), 8.51 (0.9H, d, J = 8.2 Hz), 8.60 (0.1H, dd, J = 8.3, 1.6 Hz), 8.98 (0.9H, dd, J = 4.3, 1.7 Hz), 9.03-9.06 (1.1H, m). |
| 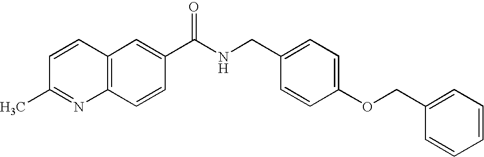 | 1H-NMR (CDCl3) δ: 2.77 (3H, s), 4.64 (2H, d, J = 5.6 Hz), 5.08 (2H, s), 6.46 (1H, s), 6.98 (2H, d, J = 8.5 Hz), 7.31-7.44 (8H, m), 8.00 (1H, dd, J = 8.9, 1.8 Hz), 8.05 (1H, d, J = 8.8 Hz), 8.11 (1H, d, J = 8.5 Hz), 8.28 (1H, d, J = 1.5 Hz). |
| 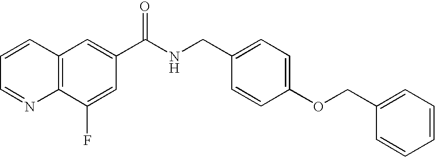 | 1H-NMR (CDCl3) δ: 4.63 (2H, d, J = 5.6 Hz), 5.08 (2H, s), 6.45 (1H, s), 6.98 (2H, d, J = 8.5 Hz), 7.31- 7.35 (3H, m), 7.37-7.44 (4H, m), 7.54 (1H, dd, J = 8.5, 4.1 Hz), 7.80 (1H, dd, J = 11.0, 1.7 Hz), 8.08 (1H, s), 8.25 (1H, d, J = 8.3 Hz), 9.04 (1H, dd, J = 4.1, 1.5 Hz). |
| 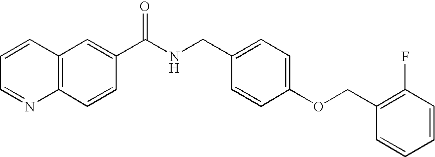 | 1H-NMR (CDCl3) δ: 4.65 (2H, d, J = 5.4 Hz), 5.15 (2H, s), 6.51 (1H, br s), 7.00 (2H, d, J = 8.5 Hz), 7.07-7.19 (2H, m), 7.27-7.35 (3H, m), 7.46-7.52 (2H, m), 8.06 (1H, d, J = 8.8, 2.0 Hz), 8.16 (1H, d, J = 8.8 Hz), 8.24 (1H, d, J = 8.8 Hz), 8.33 (1H, d, J = 1.7 Hz), 8.99 (1H, d, J = 4.3, 1.6 Hz). |
| 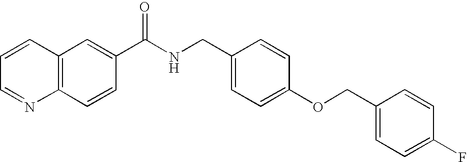 | 1H-NMR (CDCl3) δ: 4.65 (2H, d, J = 5.6 Hz), 5.03 (2H, s), 6.53 (1H, br s), 6.95-6.98 (2H, m), 7.05-7.10 (2H, m), 7.33 (2H, d, J = 8.8 Hz), 7.38-7.42 (2H, m), 7.47 (1H, dd, J = 8.3, 4.1 Hz), 8.05 (1H, d, J = 8.9, 2.0 Hz), 8.15 (1H, d, J = 8.9 Hz), 8.20-8.24 (1H, m), 8.32 (1H, d, J = 2.0 Hz), 8.98 (1H, dd, J = 4.1, 1.7 Hz). |
| 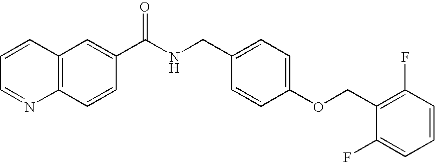 | 1H-NMR (CDCl3) δ: 4.66 (2H, d, J = 5.4 Hz), 5.13 (2H, s), 6.50 (1H, br s), 6.92-6.96 (2H, m), 7.00-7.02 (2H, m), 7.31-7.36 (3H, m), 7.47 (1H, dd, J = 8.3, 4.1 Hz), 8.06 (1H, dd, J = 8.7, 1.6 Hz), 8.15 (1H, d, J = 8.8 Hz), 8.24 (1H, d, J = 8.3 Hz), 8.32 (1H, s), 8.99 (1H, d, J = 2.7 Hz). |
| 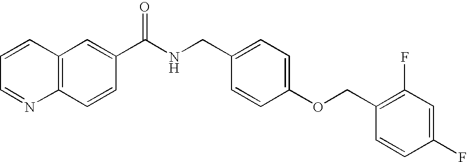 | 1H-NMR (CDCl3) δ: 4.66 (2H, d, J = 5.4 Hz), 5.08 (2H, s), 6.50 (1H, br s), 6.82-6.93 (2H, m), 6.96-6.99 (2H, m), 7.34 (2H, d, J = 8.8 Hz), 7.45-7.50 (2H, m), 8.05 (1H, dd, J = 8.9, 2.1 Hz), 8.15 (1H, d, J = 8.8 Hz), 8.24 (1H, d, J = 8.5 Hz), 8.32 (1H, d, J = 2.0 Hz), 8.99 (1H, dd, J = 4.1, 1.7 Hz). |
| 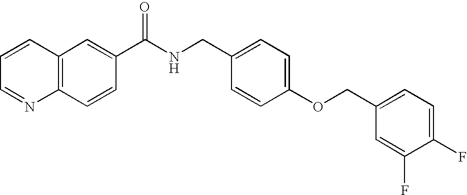 | 1H-NMR (CDCl3) δ: 4.65 (2H, d, J = 5.6 Hz), 5.02 (2H, s), 6.54 (1H, br s), 6.93-6.97 (2H, m), 7.12-7.35 (5H, m), 7.47 (1H, dd, J = 8.3, 4.2 Hz), 8.05 (1H, dd, J = 8.8, 2.1 Hz), 8.15 (1H, d, J = 8.9 Hz), 8.23 (1H, d, J = 1.7 Hz), 8.32 (1H, d, J = 1.9 Hz), 8.99 (1H, dd, J = 4.2, 1.6 Hz). |
| 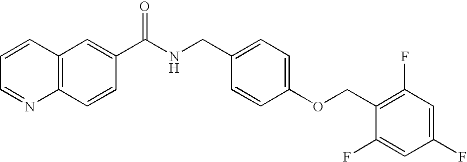 | 1H-NMR (CDCl3) δ: 4.66 (2H, d, J = 5.4 Hz), 5.07 (2H, s), 6.50 (1H, br s), 6.72 (2H, dd, J = 8.5, 7.6 Hz), 6.98-7.01 (2H, m), 7.33-7.36 (2H, m), 7.48 (1H, dd, J = 8.3, 4.1 Hz), 8.06 (1H, dd, J = 8.9, 2.1 Hz), 8.16 (1H, d, J = 8.8 Hz), 8.24 (1H, d, J = 8.5 Hz), 8.32 (1H, d, J = 1.7 Hz), 8.99 (1H, dd, J = 4.4, 1.7 Hz). |
| 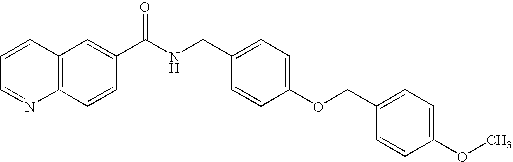 | 1H-NMR (CDCl3) δ: 3.82 (3H, s), 4.65 (2H, d, J = 5.6 Hz), 5.00 (2H, s), 6.48 (1H, br s), 6.92 (2H, d, J = 8.7 Hz), 6.97 (2H, d, J = 8.7 Hz), 7.31-7.37 (4H, m), 7.47 (1H, dd, J = 8.2, 4.1 Hz), 8.05 (1H, dd, J = 8.8, 1.8 Hz), 8.15 (1H, d, J = 8.9 Hz), 8.23 (1H, d, J = 8.5 Hz), 8.32 (1H, d, J = 1.7 Hz), 8.99 (1H, dd, J = 4.2, 1.6 Hz). |
| 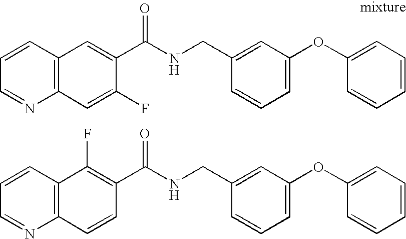 | 1H-NMR (CDCl3) δ: 4.47 (0.2H, d, J = 6.1 Hz), 4.72 (1.8H, d, J = 5.1 Hz), 6.88-6.94 (1.0H, m), 6.99-7.05 (3.0H, m), 7.08-7.14 (2.0H, m), 7.17-7.23 (1.0H, m), 7.30-7.36 (3.0H, m), 7.46 (0.9H, dd, J = 8.3, 4.1 Hz), 7.53 (0.1H, dd, J = 8.5, 4.4 Hz), 7.80 (0.9H, d, J = 13.4 Hz), 7.99 (0.1H, d, J = 8.8 Hz), 8.28 (0.9H, d, J = 7.3 Hz), 8.34 (0.1H, t, J = 8.7 Hz), 8.48 (0.1H, d, J = 7.8 Hz), 8.71 (0.9H, d, J = 8.3 Hz), 8.99 (0.9H, dd, J = 4.4, 1.7 Hz), 9.03 (0.1H, dd, J = 4.1, 1.7 Hz). |
| 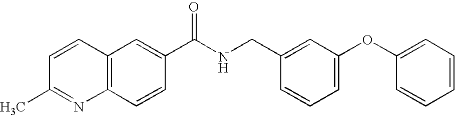 | 1H-NMR (CDCl3) δ: 2.77 (3H, s), 4.68 (2H, d, J = 5.6 Hz), 6.55 (1H, s), 6.93 (1H, dd, J = 8.2, 1.8 Hz), 7.01-7.04 (3H, m), 7.09-7.13 (2H, m), 7.30-7.36 (3H, m), 8.00 (1H, dd, J = 8.9, 1.8 Hz), 8.05 (1H, d, J = 8.8 Hz), 8.11 (1H, d, J = 8.3 Hz), 8.28 (1H, d, J = 1.5 Hz). |
| 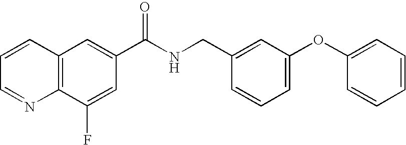 | 1H-NMR (CDCl3) δ: 4.66 (2H, d, J = 5.9 Hz), 6.82 (1H, s), 6.91 (1H, dd, J = 8.3, 1.7 Hz), 6.99-7.01 (3H, m), 7.09-7.13 (2H, m), 7.28-7.35 (3H, m), 7.52 (1H, dd, J = 8.3, 4.1 Hz), 7.79 (1H, dd, J = 10.9, 1.8 Hz), 8.08 (1H, s), 8.21 (1H, dt, J = 8.4, 1.4 Hz), 9.01 (1H, dd, J = 4.3, 1.6 Hz). |
| 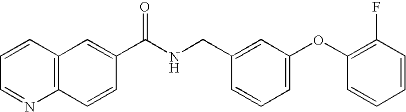 | 1H-NMR (CDCl3) δ: 4.69 (2H, d, J = 5.9 Hz), 6.60 (1H, br s), 6.89 (1H, d, J = 8.3 Hz), 7.02 (1H, s), 7.08-7.20 (5H, m), 7.30-7.34 (1H, m), 7.48 (1H, dd, J = 8.3, 4.1 Hz), 8.05 (1H, dd, J = 8.8, 2.0 Hz), 8.15 (1H, d, J = 8.5 Hz), 8.24 (1H, d, J = 8.0 Hz), 8.32 (1H, d, J = 1.5 Hz), 8.98-9.01 (1H, m). |
| 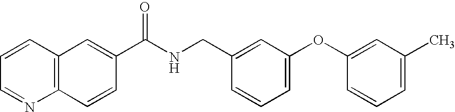 | 1H-NMR (CDCl3) δ: 2.32 (3H, s), 4.70 (2H, d, J = 5.6 Hz), 6.55 (1H, br s), 6.81-6.85 (2H, m), 6.93 (2H, d, J = 7.2 Hz), 7.04 (1H, s), 7.12 (1H, d, J = 7.5 Hz), 7.20-7.35 (2H, m), 7.48 (1H, dd, J = 8.3, 4.2 Hz), 8.05 (1H, d, J = 8.7 Hz), 8.16 (1H, d, J = 8.9 Hz), 8.24 (1H, d, J = 7.7 Hz), 8.32 (1H, s), 9.00 (1H, d, J = 2.4 Hz). |
| 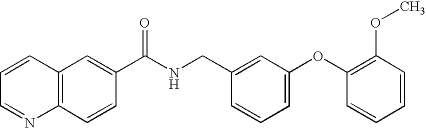 | 1H-NMR (CDCl3) δ: 3.82 (3H, s), 4.67 (2H, d, J = 5.6 Hz), 6.58 (1H, br s), 6.85 (1H, dd, J = 8.2, 1.8 Hz), 6.91-7.01 (4H, m), 7.07 (1H, d, J = 7.6 Hz), 7.15 (1H, dd, J = 7.9, 7.4 Hz), 7.26-7.30 (1H, m), 7.48 (1H, dd, J = 8.3, 4.1 Hz), 8.04 (1H, dd, J = 8.8, 1.7 Hz), 8.16 (1H, d, J = 8.8 Hz), 8.24 (1H, d, J = 7.8 Hz), 8.31 (1H, s), 8.99 (1H, d, J = 2.4 Hz). |
| 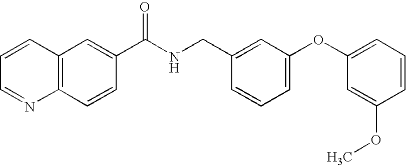 | 1H-NMR (CDCl3) δ: 3.77 (3H, s), 4.69 (2H, d, J = 3.9 Hz), 6.55-6.67 (4H, m), 6.95 (1H, dd, J = 7.8, 2.1 Hz), 7.05 (1H, t, J = 1.9 Hz), 7.14 (1H, d, J = 7.7 Hz), 7.22 (1H, dd, J = 8.1, 8.1 Hz), 7.33 (1H, dd, J = 8.0, 8.0 Hz), 7.47 (1H, dd, J = 8.2, 4.3 Hz), 8.05 (1H, dd, J = 8.8, 2.1 Hz), 8.15 (1H, d, J = 8.7 Hz), 8.23 (1H, d, J = 8.2 Hz), 8.32 (1H, d, J = 1.9 Hz), 8.96- 9.00 (1H, m). |
| 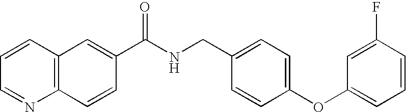 | 1H-NMR (CDCl3) δ: 4.71 (2H, d, J = 5.6 Hz), 6.61 (1H, br s), 6.70 (1H, ddd, J = 10.2, 2.4, 2.4 Hz), 6.77- 6.82 (2H, m), 7.03-7.06 (2H, m), 7.24-7.30 (1H, m), 7.38-7.41 (2H, m), 7.48 (1H, dd, J = 8.3, 4.2 Hz), 8.08 (1H, dd, J = 8.8, 2.1 Hz), 8.17 (1H, d, J = 8.9 Hz), 8.25 (1H, dd, J = 8.3, 1.1 Hz), 8.35 (1H, d, J = 1.9 Hz), 8.99 (1H, dd, J = 4.2, 1.8 Hz). |
| 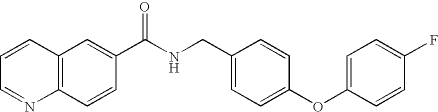 | 1H-NMR (CDCl3) δ: 4.68 (2H, d, J = 5.9 Hz), 6.53- 6.59 (1H, br m), 6.95-7.06 (6H, m), 7.36 (2H, d, J = 8.3 Hz), 7.48 (1H, dd, J = 8.3, 4.2 Hz), 8.06 (1H, dd, J = 8.8, 2.2 Hz), 8.16 (1H, d, J = 8.8 Hz), 8.24 (1H, dd, J = 8.4, 1.3 Hz), 8.34 (1H, d, J = 2.0 Hz), 8.99 (1H, dd, J = 4.4, 1.7 Hz). |
| 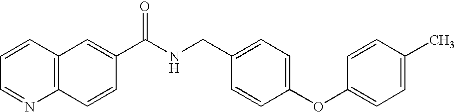 | 1H-NMR (CDCl3) δ: 2.34 (3H, s), 4.68 (2H, d, J = 5.6 Hz), 6.54 (1H, br s), 6.92 (2H, d, J = 8.5 Hz), 6.98 (2H, d, J = 8.7 Hz), 7.14 (2H, d, J = 8.2 Hz), 7.33-7.35 (2H, m), 7.47 (1H, dd, J = 8.3, 4.2 Hz), 8.06 (1H, dd, J = 8.8, 2.1 Hz), 8.16 (1H, d, J = 8.9 Hz), 8.24 (1H, d, J = 8.2 Hz), 8.33 (1H, d, J = 1.7 Hz), 8.99 (1H, dd, J = 4.1, 1.7 Hz). |
| 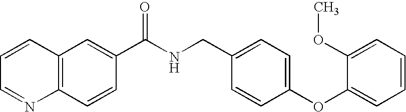 | 1H-NMR (CDCl3) δ: 3.83 (3H, s), 4.66 (2H, d, J = 5.4 Hz), 6.57 (1H, br s), 6.91-7.03 (5H, m), 7.15 (1H, dd, J = 7.2, 6.5 Hz), 7.32 (2H, d, J = 8.3 Hz), 7.47 (1H, dd, J = 8.3, 4.1 Hz), 8.06 (1H, dd, J = 8.9, 1.8 Hz), 8.15 (1H, d, J = 8.8 Hz), 8.23 (1H, d, J = 8.0 Hz), 8.32 (1H, s), 8.98 (1H, d, J = 2.4 Hz). |
| 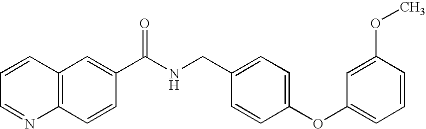 | 1H-NMR (CDCl3) δ: 3.78 (3H, s), 4.69 (2H, d, J = 5.6 Hz), 6.58-6.67 (4H, m), 7.02 (2H, d, J = 8.5 Hz), 7.21-7.26 (1H, m), 7.37 (2H, d, J = 8.5 Hz), 7.47 (1H, dd, J = 8.2, 4.3 Hz), 8.07 (1H, dd, J = 8.8, 1.7 Hz), 8.16 (1H, d, J = 8.5 Hz), 8.24 (1H, d, J = 8.3 Hz), 8.34 (1H, s), 8.99 (1H, d, J = 2.7 Hz). |
| 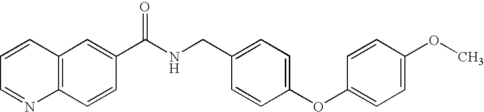 | 1H-NMR (CDCl3) δ: 3.81 (3H, s), 4.67 (2H, d, J = 5.6 Hz), 6.52 (1H, br s), 6.87-7.00 (6H, m), 7.33 (2H, d, J = 8.8 Hz), 7.47 (1H, dd, J = 8.3, 4.1 Hz), 8.06 (1H, dd, J = 8.8, 2.0 Hz), 8.16 (1H, d, J = 8.8 Hz), 8.24 (1H, d, J = 8.5 Hz), 8.33 (1H, d, J = 2.0 Hz), 8.99 (1H, dd, J = 4.1, 1.7 Hz). |
| 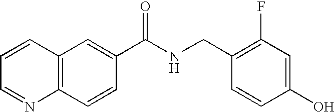 | 1H-NMR (DMSO-D6) δ: 4.46 (2H, d, J = 5.4 Hz), 6.55-6.59 (2H, m), 7.15-7.25 (2H, m), 7.61 (1H, dd, J = 8.0, 3.9 Hz), 8.08 (1H, d, J = 8.8 Hz), 8.20 (1H, d, J = 8.8 Hz), 8.47 (1H, d, J = 8.3 Hz), 8.54 (1H, s), 8.98 (1H, d, J = 3.9 Hz), 9.12-9.15 (1H, m). |
| 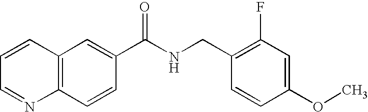 | 1H-NMR (CDCl3) δ: 3.80 (3H, s), 4.68 (2H, d, J = 5.8 Hz), 6.60 (1H, br s), 6.64-6.71 (2H, m), 7.38 (1H, dd, J = 8.6, 8.3 Hz), 7.47 (1H, dd, J = 8.3, 4.2 Hz), 8.04 (1H, dd, J = 8.8, 1.8 Hz), 8.15 (1H, d, J = 8.7 Hz), 8.24 (1H, d, J = 8.2 Hz), 8.31 (1H, d, J = 1.7 Hz), 8.98 (1H, dd, J = 4.1, 1.4 Hz). |
| 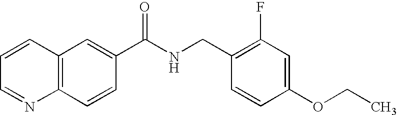 | 1H-NMR (CDCl3) δ: 1.41 (3H, t, J = 6.8 Hz), 4.01 (2H, q, J = 6.8 Hz), 4.68 (2H, d, J = 5.4 Hz), 6.62- 6.69 (3H, m), 7.35 (1H, dd, J = 8.8, 8.8 Hz), 7.47 (1H, dd, J = 7.9, 4.0 Hz), 8.05 (1H, d, J = 9.0 Hz), 8.14 (1H, d, J = 8.5 Hz), 8.23 (1H, d, J = 8.0 Hz), 8.31 (1H, s), 8.97-8.99 (1H, m). |
| 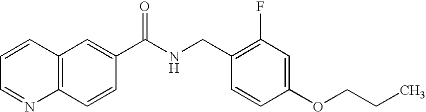 | 1H-NMR (CDCl3) δ: 1.03 (3H, t, J = 7.3 Hz), 1.76- 1.85 (2H, m), 3.90 (2H, t, J = 6.5 Hz), 4.68 (2H, d, J = 5.6 Hz), 6.60 (1H, br s), 6.62-6.70 (2H, m), 7.35 (1H, dd J = 8.5, 8.3 Hz), 7.47 (1H, dd, J = 8.0, 4.1 Hz), 8.04 (1H, d, J = 8.8 Hz), 8.15 (1H, d, J = 8.8 Hz), 8.23 (1H, d, J = 8.3 Hz), 8.30 (1H, s), 8.97-8.99 (1H, m). |
| 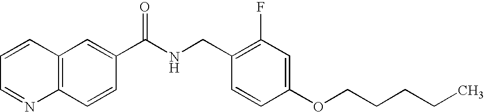 | 1H-NMR (CDCl3) δ: 0.93 (3H, t, J = 6.8 Hz), 1.34- 1.47 (4H, m), 1.74-1.81 (2H, m), 3.93 (2H, t, J = 6.6 Hz), 4.68 (2H, d, J = 5.6 Hz), 6.58 (1H, br s), 6.62- 6.70 (2H, m), 7.35 (1H, dd, J = 8.5, 8.3 Hz), 7.47 (1H, dd, J = 8.3, 4.1 Hz), 8.04 (1H, d, J = 8.8 Hz), 8.15 (1H, d, J = 8.8 Hz), 8.23 (1H, d, J = 8.3 Hz), 8.30 (1H, s), 8.98 (1H, dd, J = 4.1, 1.7 Hz). |
| 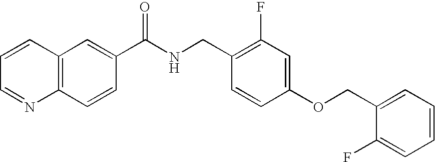 | 1H-NMR (CDCl3) δ: 4.69 (2H, d, J = 4.3 Hz), 5.12 (2H, s), 6.62 (1H, br s), 6.74-6.81 (2H, m), 7.07-7.50 (6H, m), 8.05 (1H, d, J = 8.2 Hz), 8.15 (1H, d, J = 8.7 Hz), 8.24 (1H, d, J = 8.5 Hz), 8.31 (1H, s), 8.99 (1H, s). |
| 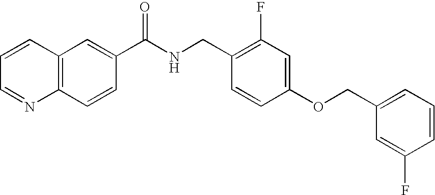 | 1H-NMR (CDCl3) δ: 4.69 (2H, d, J = 5.6 Hz), 5.05 (2H, s), 6.61 (1H, br s), 6.70-6.76 (2H, m), 7.02 (1H, dd, J = 7.6, 7.1 Hz), 7.15 (2H, dd, J = 16.9, 9.2 Hz), 7.33-7.41 (2H, m), 7.47 (1H, dd, J = 7.8, 4.2 Hz), 8.04 (1H, d, J = 9.2 Hz), 8.15 (1H, d, J = 8.9 Hz), 8.24 (1H, d, J = 8.0 Hz), 8.31 (1H, s), 8.99 (1H, s). |
| 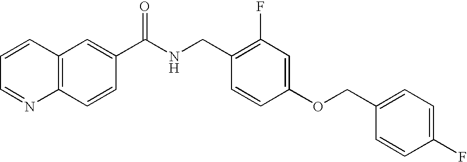 | 1H-NMR (CDCl3) δ: 4.69 (2H, d, J = 5.8 Hz), 5.01 (2H, s), 6.59 (1H, br s), 6.70-6.77 (2H, m), 7.08 (2H, dd, J = 8.7, 8.7 Hz), 7.36-7.41 (3H, m), 7.47 (1H, dd, J = 8.3, 4.2 Hz), 8.04 (1H, dd, J = 8.9, 1.9 Hz), 8.15 (1H, d, J = 8.7 Hz), 8.24 (1H, d, J = 8.5 Hz), 8.31 (1H, d, J = 1.9 Hz), 8.99 (1H, dd, J = 4.1, 1.7 Hz). |
| 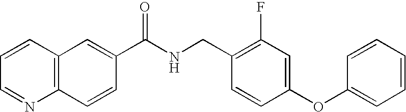 | 1H-NMR (CDCl3) δ: 4.71 (2H, d, J = 5.9 Hz), 6.71- 6.79 (3H, m), 7.01-7.05 (2H, m), 7.14-7.18 (1H, m), 7.34-7.43 (3H, m), 7.47 (1H, dd, J = 8.3, 4.4 Hz), 8.05 (1H, dd, J = 8.9, 2.1 Hz), 8.15 (1H, d, J = 8.8 Hz), 8.23 (1H, dd, J = 8.5, 1.5 Hz), 8.32 (1H, d, J = 1.7 Hz), 8.98 (1H, dd, J = 4.1, 1.7 Hz). |
| 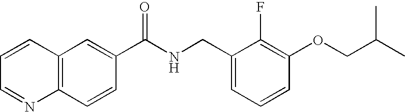 | 1H-NMR (CDCl3) δ: 1.05 (6H, d, J = 6.3 Hz), 2.08- 2.19 (1H, m), 3.79 (2H, d, J = 6.3 Hz), 4.76 (2H, d, J = 5.9 Hz), 6.68 (1H, br s), 6.90-7.08 (3H, m), 7.47 (1H, dd, J = 8.0, 4.1 Hz), 8.06 (1H, d, J = 8.3 Hz), 8.15 (1H, d, J = 8.5 Hz), 8.24 (1H, d, J = 8.5 Hz), 8.31 (1H, s), 8.98 (1H, s). |
| 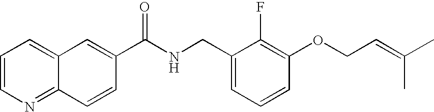 | 1H-NMR (CDCl3) δ: 1.77 (6H, d, J = 18.3 Hz), 4.59 (2H, d, J = 6.8 Hz), 4.76 (2H, dd, J = 5.9, 1.0 Hz), 5.49-5.53 (1H, m), 6.68 (1H, s), 6.92-6.96 (1H, m), 7.00-7.07 (2H, m), 7.47 (1H, dd, J = 8.3, 4.4 Hz), 8.06 (1H, dd, J = 8.9, 2.1 Hz), 8.15 (1H, d, J = 8.8 Hz), 8.23 (1H, dd, J = 8.5, 1.0 Hz), 8.30 (1H, d, J = 2.0 Hz), 8.98 (1H, dd, J = 4.3, 1.8 Hz). |
| 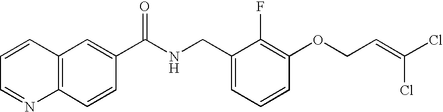 | 1H-NMR (CDCl3) δ: 4.73 (2H, d, J = 6.3 Hz), 4.77 (2H, d, J = 6.1 Hz), 6.19 (1H, t, J = 6.2 Hz), 6.71 (1H, s), 6.90-6.94 (1H, m), 7.05-7.11 (2H, m), 7.47 (1H, dd, J = 8.3, 4.1 Hz), 8.06 (1H, dd, J = 8.8, 2.0 Hz), 8.15 (1H, d, J = 8.8 Hz), 8.24 (1H, d, J = 8.0 Hz), 8.32 (1H, d, J = 2.0 Hz), 8.99 (1H, dd, J = 4.1, 1.5 Hz). |
| 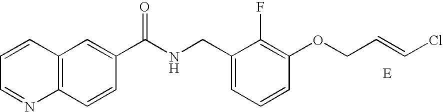 | 1H-NMR (CDCl3) δ: 4.58 (2H, dd, J = 6.1, 1.5 Hz), 4.76 (2H, d, J = 5.1 Hz), 6.18 (1H, dt, J = 13.3, 6.0 Hz), 6.41 (1H, dt, J = 13.4, 1.5 Hz), 6.75 (1H, s), 6.89-6.93 (1H, m), 7.03-7.09 (2H, m), 7.46 (1H, dd, J = 8.3, 4.4 Hz), 8.06 (1H, dd, J = 8.8, 2.0 Hz), 8.14 (1H, d, J = 8.8 Hz), 8.22 (1H, dd, J = 8.5, 1.0 Hz), 8.31 (1H, d, J = 2.0 Hz), 8.98 (1H, dd, J = 4.1, 1.7 Hz). |
| 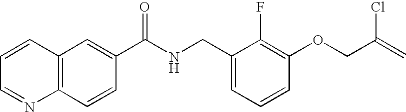 | 1H-NMR (CDCl3) δ: 4.65 (2H, s), 4.77 (2H, d, J = 4.9 Hz), 5.46-5.47 (1H, m), 5.60-5.61 (1H, m), 6.69 (1H, s), 6.94 (1H, td, J = 7.9, 2.0 Hz), 7.04-7.12 (2H, m), 7.47 (1H, dd, J = 8.3, 4.4 Hz), 8.06 (1H, dd, J = 8.8, 2.0 Hz), 8.15 (1H, d, J = 8.8 Hz), 8.24 (1H, d, J = 8.0 Hz), 8.32 (1H, d, J = 1.7 Hz), 8.99 (1H, dd, J = 4.4, 1.7 Hz). |
| 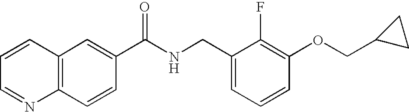 | 1H-NMR (CDCl3) δ: 0.37 (2H, d, J = 3.7 Hz), 0.66 (2H, d, J = 6.8 Hz), 1.29-1.35 (1H, m), 3.88 (2H, d, J = 6.6 Hz), 4.76 (2H, d, J = 4.9 Hz), 6.71 (1H, br s), 6.89-6.93 (1H, m), 7.01-7.06 (2H, m), 7.47 (1H, dd, J = 8.4, 3.8 Hz), 8.06 (1H, d, J = 8.5 Hz), 8.15 (1H, d, J = 8.3 Hz), 8.24 (1H, d, J = 7.8 Hz), 8.31 (1H, s), 8.99 (1H, s). |
| 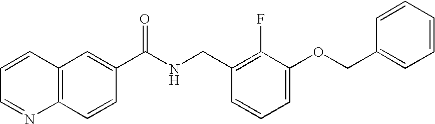 | 1H-NMR (CDCl3) δ: 4.75 (2H, d, J = 5.9 Hz), 5.13 (2H, s), 6.77-6.89 (1H, m), 6.91-7.08 (3H, m), 7.32- 7.46 (6H, m), 8.05 (1H, dd, J = 8.8, 2.0 Hz), 8.13 (1H, d, J = 8.8 Hz), 8.18-8.22 (1H, m), 8.30 (1H, d, J = 1.7 Hz), 8.96 (1H, dd, J = 4.1, 1.5 Hz). |
| 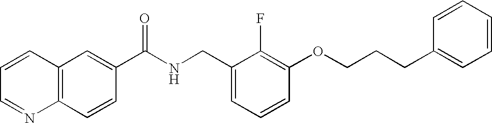 | 1H-NMR (CDCl3) δ: 2.12-2.19 (2H, m), 2.84 (2H, t, J = 7.7 Hz), 4.04 (2H, t, J = 6.3 Hz), 4.77 (2H, d, J = 5.4 Hz), 6.66 (1H, br s), 6.87-6.93 (1H, m), 7.00-7.06 (2H, m), 7.18-7.31 (5H, m), 7.47 (1H, dd, J = 8.3, 4.1 Hz), 8.06 (1H, dd, J = 8.9, 1.8 Hz), 8.16 (1H, d, J = 8.8 Hz), 8.25 (1H, d, J = 8.0 Hz), 8.32 (1H, s), 8.99 (1H, dd, J = 4.1, 1.7 Hz). |
| 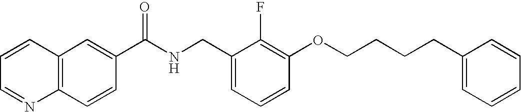 | 1H-NMR (CDCl3) δ: 1.79-1.91 (4H, m), 2.70 (2H, t, J = 7.2 Hz), 4.04 (2H, t, J = 6.0 Hz), 4.76 (2H, d, J = 5.9 Hz), 6.67 (1H, s), 6.88-6.93 (1H, m), 7.00-7.06 (2H, m), 7.16-7.21 (3H, m), 7.26-7.30 (2H, m), 7.46 (1H, dd, J = 8.3, 4.1 Hz), 8.05 (1H, dd, J = 8.8, 2.0 Hz), 8.14 (1H, d, J = 9.0 Hz), 8.23 (1H, dd, J = 8.3, 1.0 Hz), 8.31 (1H, d, J = 1.7 Hz), 8.98 (1H, dd, J = 4.4, 1.7 Hz). |
| 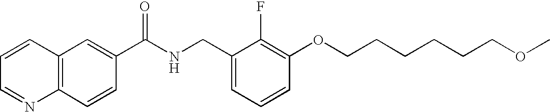 | 1H-NMR (CDCl3) δ: 1.36-1.64 (6H, m), 1.80-1.88 (2H, m), 3.33 (3H, s), 3.38 (2H, t, J = 6.5 Hz), 4.04 (2H, t, J = 6.6 Hz), 4.76 (2H, d, J = 5.6 Hz), 6.67 (1H, br s), 6.91-6.95 (1H, m), 7.00-7.07 (2H, m), 7.47 (1H, dd, J = 8.2, 4.1 Hz), 8.06 (1H, dd, J = 8.7, 1.9 Hz), 8.15 (1H, dd, J = 8.7 Hz), 8.24 (1H, d, J = 8.0 Hz), 8.31 (1H, d, J = 1.4 Hz), 8.99 (1H, dd, J = 4.1, 1.4 Hz). |
| 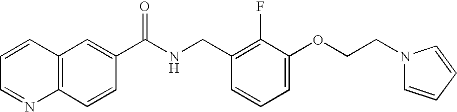 | 1H-NMR (CDCl3) δ: 4.26 (2H, t, J = 4.9 Hz), 4.31 (2H, t, J = 5.0 Hz), 4.75 (2H, d, J = 5.9 Hz), 6.17 (2H, t, J = 2.0 Hz), 6.71 (1H, s), 6.78-6.82 (3H, m), 6.98-7.06 (2H, m), 7.46 (1H, dd, J = 8.3, 4.4 Hz), 8.05 (1H, dd, J = 8.8, 2.0 Hz), 8.14 (1H, d, J = 8.8 Hz), 8.22 (1H, d, J = 7.8 Hz), 8.31 (1H, d, J = 1.7 Hz), 8.98 (1H, dd, J = 4.3, 1.3 Hz). |
| 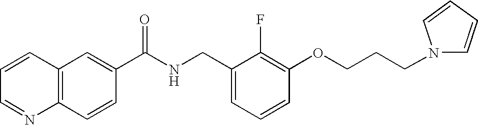 | 1H-NMR (CDCl3) δ: 2.21-2.28 (2H, m), 3.95 (2H, t, J = 5.7 Hz), 4.15 (2H, t, J = 6.6 Hz), 4.78 (2H, dd, J = 5.9, 0.7 Hz), 6.14 (2H, t, J = 2.1 Hz), 6.66-6.69 (3H, m), 6.84-6.89 (1H, m), 7.03-7.05 (2H, m), 7.47 (1H, dd, J = 8.3, 4.1 Hz), 8.06 (1H, dd, J = 8.9, 2.1 Hz), 8.15 (1H, dd, J = 8.8 Hz), 8.24 (1H, dd, J = 8.5, 7.0 Hz), 8.32 (1H, dd, J = 1.7 Hz), 8.99 (1H, dd, J = 4.1, 1.7 Hz). |
| 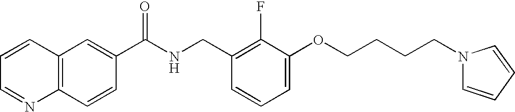 | 1H-NMR (CDCl3) δ: 1.77-1.84 (2H, m), 1.96-2.03 (2H, m), 3.97-4.02 (4H, m), 4.76 (2H, d, J = 5.9 Hz), 6.14 (2H, t, J = 2.1 Hz), 6.64 (1H, s), 6.68 (2H, t, J = 2.2 Hz), 6.86-6.91 (1H, m), 7.01-7.07 (2H, m), 7.47 (1H, dd, J = 8.3, 4.4 Hz), 8.05 (1H, dd, J = 8.8, 2.0 Hz), 8.15 (1H, dd, J = 8.8 Hz), 8.24 (1H, d, J = 8.0 Hz), 8.31 (1H, d, J = 2.0 Hz), 8.99 (1H, dd, J = 4.1, 1.7 Hz). |
| 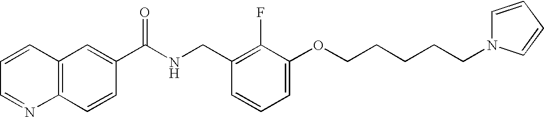 | 1H-NMR (CDCl3) δ: 1.45-1.53 (2H, m), 1.81-1.89 (4H, m), 3.91 (2H, t, J = 7.1 Hz), 4.01 (2H, t, J = 6.3 Hz), 4.76 (2H, d, J = 4.9 Hz), 6.13 (2H, t, J = 2.2 Hz), 6.65 (2H, t, J = 2.1 Hz), 6.68 (1H, s), 6.87-6.92 (1H, m), 7.00-7.06 (2H, m), 7.47 (1H, dd, J = 8.3, 4.1 Hz), 8.05 (1H, dd, J = 8.8, 2.0 Hz), 8.15 (1H, d, J = 8.8 Hz), 8.22-8.24 (1H, m), 8.31 (1H, d, J = 2.0 Hz), 8.99 (1H, dd, J = 4.1, 1.7 Hz). |
| 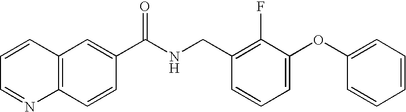 | 1H-NMR (CDCl3) δ: 4.80 (2H, d, J = 5.8 Hz), 6.82 (1H, br s), 6.97-7.02 (3H, m), 7.06-7.13 (2H, m), 7.22-7.27 (1H, m), 7.33 (2H, dd, J = 8.1, 7.8 Hz), 7.47 (1H, dd, J = 8.0, 3.9 Hz), 8.07 (1H, dd, J = 8.7, 1.9 Hz), 8.15 (1H, d, J = 8.7 Hz), 8.23 (1H, d, J = 8.2 Hz), 8.32 (1H, d, J = 1.9 Hz), 8.99 (1H, t, J = 2.1 Hz). |
| 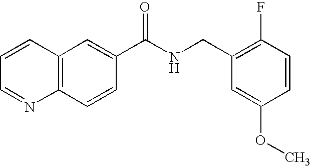 | 1H-NMR (CDCl3) δ: 3.79 (3H, s), 4.73 (2H, d, J = 5.4 Hz), 6.64 (1H, br s), 6.78-6.82 (1H, m), 6.97-7.04 (2H, m), 7.48 (1H, dd, J = 8.0, 4.4 Hz), 8.06 (1H, d, J = 8.8 Hz), 8.16 (1H, d, J = 8.8 Hz), 8.25 (1H, d, J = 8.5 Hz), 8.32 (1H, s), 8.99 (1H, d, J = 4.1 Hz). |
| 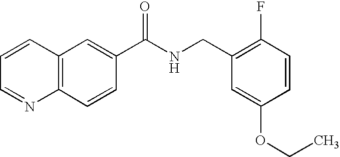 | 1H-NMR (CDCl3) δ: 7.39 (3H, t, J = 6.7 Hz), 3.99 (2H, q, J = 6.8 Hz), 4.72 (2H, d, J = 4.9 Hz), 6.70 (1H, br s), 6.76-6.81 (1H, m), 6.96-7.02 (2H, m), 7.45-7.50 (1H, m), 8.06 (1H, d, J = 7.3 Hz), 8.16 (1H, d, J = 8.5 Hz), 8.24 (1H, d, J = 9.0 Hz), 8.32 (1H, s), 8.98 (1H, s). |
| 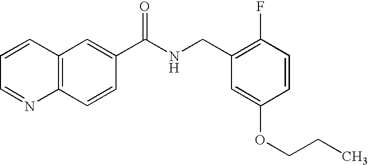 | 1H-NMR (CDCl3) δ: 0.97-1.06 (3H, m), 1.60-1.80 (2H, m), 3.86-3.90 (2H, m), 4.71-4.73 (2H, m), 6.69- 6.80 (2H, m), 6.91-7.02 (2H, m), 7.43-7.50 (1H, m), 8.03-8.08 (1H, m), 8.12-8.20 (1H, m), 8.22-8.27 (1H, m), 8.30-8.36 (1H, m), 8.98 (1H, s). |
| 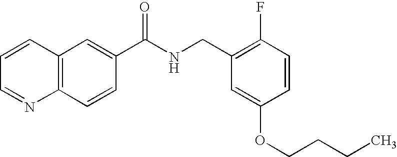 | 1H-NMR (CDCl3) δ: 0.93-0.99 (3H, m), 1.46-1.76 (4H, m), 3.92 (2H, t, J = 5.9 Hz), 4.72 (2H, d, J = 4.1 Hz), 6.68 (1H, br s), 6.74-6.82 (1H, m), 6.93-7.02 (2H, m), 7.46-7.50 (1H, m), 8.06 (1H, d, J = 8.5 Hz), 8.16 (1H, d, J = 8.0 Hz), 8.25 (1H, d, J = 8.3 Hz), 8.32 (1H, s), 8.99 (1H, s). |
| 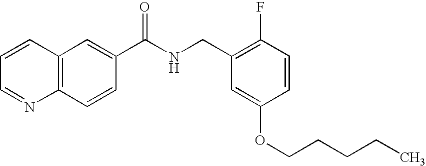 | 1H-NMR (CDCl3) δ: 0.92 (3H, t, J = 6.7 Hz), 1.30- 1.45 (4H, m), 1.70-1.79 (2H, m), 3.91 (2H, t, J = 6.6 Hz), 4.72 (2H, d, J = 5.9 Hz), 6.63 (1H, br s), 6.76- 6.82 (1H, m), 6.96-7.02 (2H, m), 7.48 (1H, dd, J = 8.2, 4.0 Hz), 8.06 (1H, d, J = 8.5 Hz), 8.16 (1H, d, J = 8.5 Hz), 8.25 (1H, d, J = 7.6 Hz), 8.32 (1H, s), 8.99 (1H, d, J = 3.4 Hz). |
| 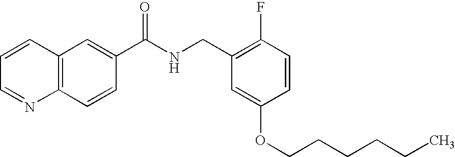 | 1H-NMR (CDCl3) δ: 0.85-0.91 (3H, m), 1.30-1.75 (8H, m), 3.91 (2H, t, J = 5.5 Hz), 4.72 (2H, d, J = 4.9 Hz), 6.67 (1H, br s), 6.78-6.81 (1H, m), 6.94-7.01 (2H, m), 7.46-7.50 (1H, m), 8.06 (1H, d, J = 7.3 Hz), 8.16 (1H, d, J = 7.6 Hz), 8.24 (1H, d, J = 7.1 Hz), 8.32 (1H, s), 8.99 (1H, s). |
| 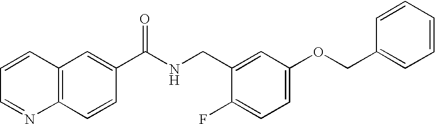 | 1H-NMR (CDCl3) δ: 4.72 (2H, d, J = 5.6 Hz), 5.02 (2H, s), 6.63-6.72 (1H, m), 6.83-6.89 (1H, m), 6.96- 7.09 (2H, m), 7.26-7.49 (6H, m), 8.05 (1H, dd, J = 8.4, 1.7 Hz), 8.16 (1H, d, J = 9.0 Hz), 8.24 (1H, d, J = 8.4 Hz), 8.31 (1H, s), 8.97-9.03 (1H, m). |
| 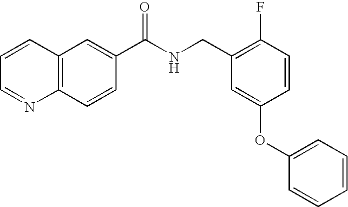 | 1H-NMR (CDCl3) δ: 4.72 (2H, d, J = 5.6 Hz), 6.74- 6.83 (1H, m), 6.88-6.93 (1H, m), 6.96 (2H, d, J = 7.5 Hz), 7.01-7.13 (3H, m), 7.29-7.33 (2H, m), 7.44-7.48 (1H, m), 8.03 (1H, d, J = 8.9 Hz), 8.13 (1H, dd, J = 8.7, 4.3 Hz), 8.21 (1H, t, J = 6.4 Hz), 8.29 (1H, s), 8.96-8.99 (1H, m). |
| 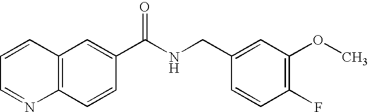 | 1H-NMR (CDCl3) δ: 3.90 (3H, s), 4.67 (2H, d, J = 5.6 Hz), 6.58 (1H, br s), 6.90-6.93 (1H, m), 7.01-7.09 (2H, m), 7.48 (1H, dd, J = 8.3, 4.2 Hz), 8.06 (1H, dd, J = 8.9, 1.9 Hz), 8.17 (1H, d, J = 8.9 Hz), 8.25 (1H, d, J = 8.2 Hz), 8.34 (1H, d, J = 1.7 Hz), 9.00 (1H, dd, J = 4.2, 1.6 Hz). |
| 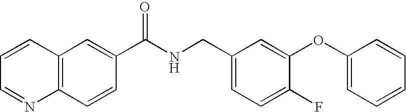 | 1H-NMR (CDCl3) δ: 4.63 (2H, d, J = 5.6 Hz), 6.72 (1H, br s), 6.96 6.99 (2H, m), 7.05-7.19 (4H, m), 7.29-7.34 (2H, m), 7.46 (1H, dd, J = 8.3, 4.3 Hz), 8.02 (1H, dd, J = 8.8, 2.1 Hz), 8.13 (1H, d, J = 8.8 Hz), 8.20 (1H, d, J = 8.3 Hz), 8.29 (1H, d, J = 2.0 Hz), 8.98 (1H, dd, J = 4.3, 1.6 Hz). |
| 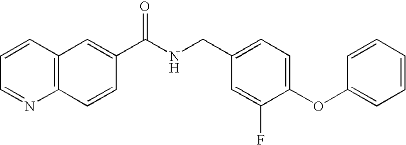 | 1H-NMR (CDCl3) δ: 4.70 (2H, d, J = 5.8 Hz), 6.66 (1H, br s), 6.96-7.14 (5H, m), 7.22-7.35 (4H, m), 7.49 (1H, dd, J = 8.2, 3.9 Hz), 8.18 (1H, d, J = 8.7 Hz), 8.25 (1H, d, J = 8.5 Hz), 8.35-8.37 (1H, m), 9.00 (1H, d, J = 2.7 Hz). |
| 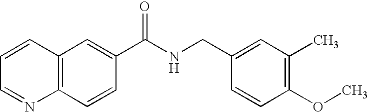 | 1H-NMR (CDCl3) δ: 2.23 (3H, s), 3.84 (3H, s), 4.62 (2H, d, J = 5.6 Hz), 6.46 (1H, br s), 6.82 (1H, d, J = 8.0 Hz), 7.17-7.21 (2H, m), 7.47 (1H, dd, J = 8.3, 4.2 Hz), 8.06 (1H, d, J = 8.7 Hz), 8.15 (1H, d, J = 8.7 Hz), 8.24 (1H, d, J = 8.2 Hz), 8.33 (1H, s), 8.99 (1H, d, J = 4.1 Hz). |
| 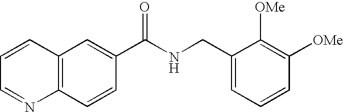 | 1H-NMR (CDCl3) δ: 3.90 (3H, s), 3.94 (3H, s), 4.73 (2H, d, J = 5.8 Hz), 6.88 (1H, br s), 6.91 (1H, dd, J = 8.0, 1.4 Hz), 7.00-7.09 (2H, m), 7.46 (1H, dd, J = 8.3, 4.2 Hz), 8.05 (1H, dd, J = 8.7, 1.9 Hz), 8.13 (1H, d, J = 8.9 Hz), 8.23 (1H, d, J = 7.7 Hz), 8.30 (1H, d, J = 1.7 Hz), 8.98 (1H, dd, J = 4.1, 7.4 Hz). |
| 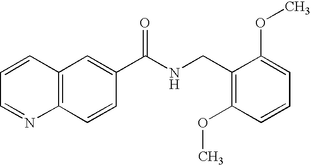 | 1H-NMR (CDCl3) δ: 3.89 (6H, s), 4.82 (2H, d, J = 5.3 Hz), 6.61 (2H, d, J = 8.2 Hz), 6.74 (1H, br s), 7.25-7.30 (1H, m), 7.45 (1H, dd, J = 8.2, 4.1 Hz), 7.99-8.03 (1H, m), 8.12 (1H, d, J = 8.5 Hz), 8.23 (1H, d, J = 8.2 Hz), 8.30-8.31 (1H, m), 8.96-8.98 (1H, m). |
| 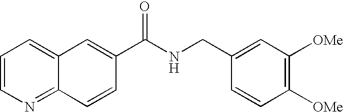 | 1H-NMR (CDCl3) δ: 3.88 (6H, s), 4.65 (2H, d, J = 5.6 Hz), 6.58 (1H, br s), 6.85-6.96 (3H, m), 7.47 (1H, dd, J = 8.4, 4.3 Hz), 8.06 (1H, dd, J = 8.8, 2.0 Hz), 8.15 (1H, d, J = 8.8 Hz), 8.23 (1H, dd, J = 8.5, 1.0 Hz), 8.33 (1H, d, J = 2.0 Hz), 8.98 (1H, dd, J = 4.3, 1.8 Hz). |
| 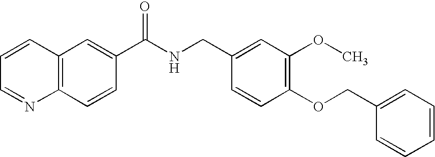 | 1H-NMR (CDCl3) δ: 3.80 (3H, s), 4.57 (2H, d, J = 5.6 Hz), 5.10 (2H, s), 6.81 (2H, d, J = 0.7 Hz), 6.89 (1H, s), 7.02 (1H, t, J = 5.3 Hz), 7.25-7.40 (6H, m), 8.04 (1H, dd, J = 8.8, 1.8 Hz), 8.07 (1H, d, J = 8.7 Hz), 8.12 (1H, dd, J = 8.3, 1.6 Hz), 8.30 (1H, s), 8.92 (1H, dd, J = 4.3, 1.7 Hz). |
| 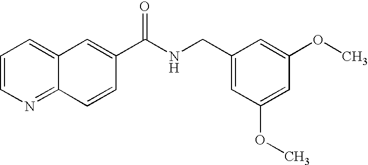 | 1H-NMR (CDCl3) δ: 3.80 (6H, s), 4.65 (2H, d, J = 5.6 Hz), 6.41 (1H, t, J = 2.2 Hz), 6.54-6.60 (1H, m), 6.54 (2H, d, J = 2.2 Hz), 7.47 (1H, dd, J = 8.2, 4.2 Hz), 8.07 (1H, dd, J = 8.8, 1.9 Hz), 8.16 (1H, d, J = 8.8 Hz), 8.24 (1H, dd, J = 8.2, 1.0 Hz), 8.33 (1H, d, J = 1.9 Hz), 8.99 (1H, dd, J = 4.2, 1.7 Hz). |
| 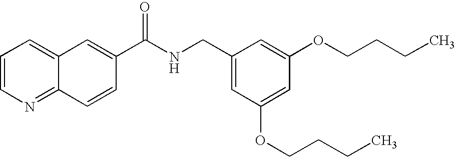 | 1H-NMR (CDCl3) δ: 0.96 (6H, t, J = 7.5 Hz), 1.43- 1.52 (4H, m), 1.71-7.78 (4H, m), 3.94 (4H, t, J = 6.4 Hz), 4.63 (2H, d, J = 5.6 Hz), 6.40 (1H, t, J = 2.2 Hz), 6.51 (2H, d, J = 2.2 Hz), 6.52-6.57 (1H, br m), 7.47 (1H, dd, J = 8.2, 4.2 Hz), 8.07 (1H, dd, J = 8.8, 1.9 Hz), 8.15 (1H, d, J = 8.8 Hz), 8.23 (1H, dd, J = 8.2, 1.3 Hz), 8.33 (1H, d, J = 1.9 Hz), 8.99 (1H, dd, J = 4.2, 1.3 Hz). |
| 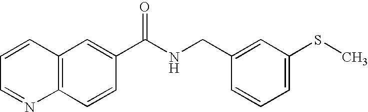 | 1H-NMR (CDCl3) δ: 2.49 (3H, s), 4.68 (2H, d, J = 5.6 Hz), 6.59 (1H, s), 7.18 (2H, dd, J = 15.0, 8.0 Hz), 7.26-7.32 (2H, m), 7.48 (1H, dd, J = 8.2, 4.1 Hz), 8.07 (1H, dd, J = 8.8, 2.1 Hz), 8.16 (1H, d, J = 8.7 Hz), 8.24 (1H, d, J = 8.2 Hz), 8.33 (1H, d, J = 1.7 Hz), 8.99 (1H, dd, J = 4.2, 1.8 Hz). |
| 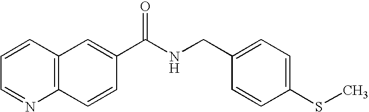 | 1H-NMR (CDCl3) δ: 2.49 (3H, s), 4.67 (2H, d, J = 5.6 Hz), 6.56 (1H, br s), 7.25-7.34 (4H, m), 7.47 (1H, dd, J = 8.3, 4.4 Hz), 8.06 (1H, dd, J = 8.8, 2.0 Hz), 8.15 (1H, d, J = 8.8 Hz), 8.23 (1H, d, J = 8.5 Hz), 8.32 (1H, d, J = 2.0 Hz), 8.99 (1H, dd, J = 4.1, 1.7 Hz). |
| 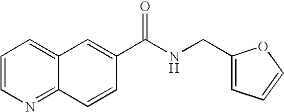 | 1H-NMR (CDCl3) δ: 4.71 (2H, d, J = 5.4 Hz), 6.33- 6.37 (2H, m), 6.67 (1H, br s), 7.40 (1H, dd, J = 1.7, 0.7 Hz), 7.47 (1H, dd, J = 8.3, 4.1 Hz), 8.06 (1H, dd, J = 8.8, 2.0 Hz), 8.15 (1H, d, J = 8.8 Hz), 8.23 (1H, d, J = 8.3 Hz), 8.32 (1H, d, J = 1.7 Hz), 8.98 (1H, dd, J = 4.1, 1.7 Hz). |
| 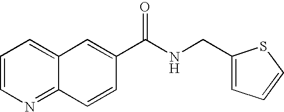 | 1H-NMR (CDCl3) δ: 4.87 (2H, d, J = 5.1 Hz), 6.88- 7.07 (3H, m), 7.24-7.29 (1H, m), 7.44 (1H, dd, J = 7.9, 4.0 Hz), 8.04-8.19 (3H, m), 8.31 (1H, s), 8.95 (1H, d, J = 2.4 Hz). |
| 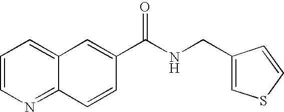 | 1H-NMR (CDCl3) δ: 4.72 (2H, d, J = 5.6 Hz), 6.59 (1H, br s), 7.14 (1H, dd, J = 4.9, 1.2 Hz), 7.26-7.27 (1H, m), 7.33-7.36 (1H, m), 7.47 (1H, dd, J = 8.3, 4.4 Hz), 8.05 (1H, dd, J = 8.8, 2.0 Hz), 8.15 (1H, d, J = 8.8 Hz), 8.23 (1H, d, J = 8.3 Hz), 8.32 (1H, d, J = 2.0 Hz), 8.98 (1H, dd, J = 4.1, 1.7 Hz). |
| 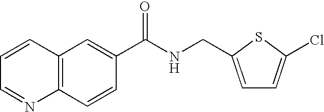 | 1H-NMR (CDCl3) δ: 4.77 (2H, d, J = 5.6 Hz), 6.66 (1H, br s), 6.79 (1H, d, J = 3.9 Hz), 6.85 (1H, d, J = 3.7 Hz), 7.48 (1H, dd, J = 8.3, 4.1 Hz), 8.05 (1H, dd, J = 8.9, 2.1 Hz), 8.16 (1H, d, J = 8.8 Hz), 8.25 (1H, d, J = 8.0 Hz), 8.33 (1H, d, J = 2.0 Hz), 8.99 (1H, dd, J = 4.1, 1.7 Hz). |
| 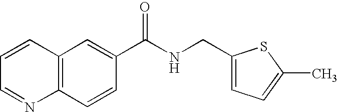 | 1H-NMR (CDCl3) δ: 2.47 (3H, s), 4.79 (2H, d, J = 5.4 Hz), 6.54 (1H, br s), 6.62-6.63 (1H, m), 6.86 (1H, d, J = 3.4 Hz), 7.47 (1H, dd, J = 8.3, 4.4 Hz), 8.05 (1H, dd, J = 8.8, 2.2 Hz), 8.15 (1H, d, J = 8.8 Hz), 8.24 (1H, d, J = 7.8 Hz), 8.32 (1H, d, J = 2.0 Hz), 8.99 (1H, dd, J = 4.1, 1.7 Hz). |
| 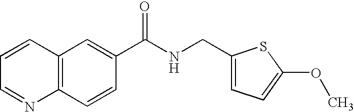 | 1H-NMR (CDCl3) δ: 3.87 (3H, s), 4.71 (2H, d, J = 5.4 Hz), 6.05 (1H, d, J = 3.7 Hz), 6.64 (1H, br s), 6.69 (1H, d, J = 3.7 Hz), 7.47 (1H, dd, J = 8.3, 4.4 Hz), 8.05 (1H, dd, J = 8.8, 2.0 Hz), 8.14 (1H, d, J = 8.8 Hz), 8.23 (1H, d, J = 7.6 Hz), 8.32 (1H, d, J = 1.7 Hz), 8.98 (1H, dd, J = 4.1, 1.7 Hz). |
| 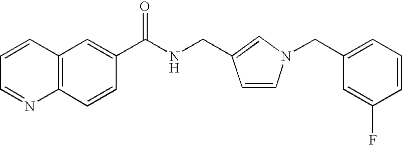 | 1H-NMR (CDCl3) δ: 4.56 (2H, d, J = 5.1 Hz), 5.03 (2H, s), 6.23-6.24 (1H, m), 6.37 (1H, s), 6.66-6.68 (1H, m), 6.72-6.73 (1H, m), 6.80-6.83 (1H, m), 6.93 (1H, dd, J = 7.6, 0.7 Hz), 6.99 (1H, td, J = 8.3, 2.3 Hz), 7.31 (1H, td, J = 7.9, 5.9 Hz), 7.46 (1H, dd, J = 8.3, 4.4 Hz), 8.05 (1H, dd, J = 8.8, 2.0 Hz), 8.14 (1H, d, J = 8.8 Hz), 8.23 (1H, dd, J = 8.4, 1.1 Hz), 8.31 (1H, d, J = 2.0 Hz), 8.98 (1H, dd, J = 4.1, 1.7 Hz). |
| 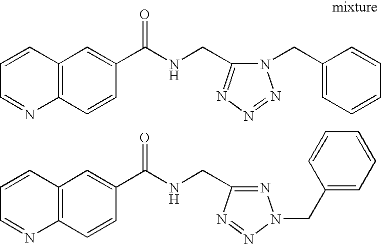 | 1H-NMR (CDCl3) δ: 4.86 (1H, d, J = 5.9 Hz), 4.97 (1H, d, J = 5.4 Hz), 5.74 (1H, s), 5.84 (1H, s), 7.29- 7.45 (7H, m), 7.99-8.18 (3H, m), 8.28-8.33 (1H, m), 8.95-8.96 (1H, m). |
| 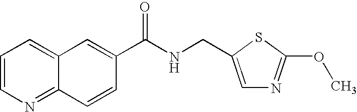 | 1H-NMR (CDCl3) δ: 4.03 (3H, s), 4.70 (2H, d, J = 5.8 Hz), 7.01-7.06 (1H, m), 7.04 (1H, s), 7.45 (1H, dd, J = 8.4, 4.3 Hz), 8.04 (1H, dd, J = 8.8, 1.9 Hz), 8.12 (1H, d, J = 8.8 Hz), 8.18 (1H, dd, J = 8.4, 1.4 Hz), 8.31 (1H, d, J = 1.9 Hz), 8.97 (1H, dd, J = 4.3, 1.7 Hz). |
| 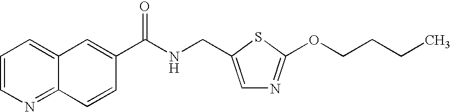 | 1H-NMR (CDCl3) δ: 0.96 (3H, t, J = 7.4 Hz), 1.41- 1.51 (2H, m), 1.73-1.81 (2H, m), 4.36 (2H, t, J = 6.5 Hz), 4.72 (2H, d, J = 5.8 Hz), 6.61-6.66 (1H, m), 7.07 (1H, d, J = 0.7 Hz), 7.48 (1H, dd, J = 8.2, 4.2 Hz), 8.04 (1H, dd, J = 9.0, 1.9 Hz), 8.16 (1H, d, J = 9.0 Hz), 8.24 (1H, dd, J = 8.2, 1.1 Hz), 8.32 (1H, d, J = 1.9 Hz), 9.00 (1H, dd, J = 4.2, 1.4 Hz). |
| 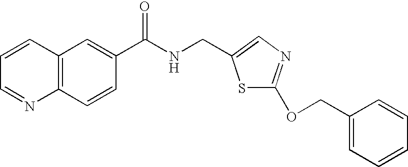 | 1H-NMR (CDCl3) δ: 4.72 (2H, d, J = 5.8 Hz), 5.42 (2H, s), 6.58-6.64 (1H, m), 7.10 (1H, s), 7.33-7.49 (6H, m), 8.03 (1H, dd, J = 8.7, 1.9 Hz), 8.16 (1H, d, J = 8.7 Hz), 8.24 (1H, d, J = 8.2 Hz), 8.31 (1H, d, J = 1.9 Hz), 8.99 (1H, dd, J = 4.1, 1.7 Hz). |
| 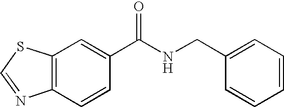 | 1H-NMR (CDCl3) δ: 4.69 (2H, d, J = 5.6 Hz), 6.55 (1H, br s), 7.30-7.42 (5H, m), 7.88 (1H, dd, J = 8.5, 1.7 Hz), 8.15 (1H, d, J = 8.5 Hz), 8.50 (1H, d, J = 1.5 Hz), 9.11 (1H, s). |
| 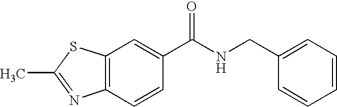 | 1H-NMR (CDCl3) δ: 2.86 (3H, s), 4.68 (2H, d, J = 5.6 Hz), 6.46 (1H, s), 7.29-7.38 (5H, m), 7.81 (1H, dd, J = 8.4, 1.8 Hz), 7.96 (1H, d, J = 8.5 Hz), 8.35 (1H, d, J = 1.7 Hz). |
| 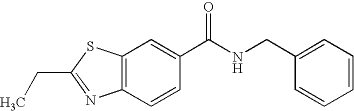 | 1H-NMR (CDCl3) δ: 1.49 (3H, t, J = 7.6 Hz), 3.17 (2H, q, J = 7.6 Hz), 4.68 (2H, d, J = 5.6 Hz), 6.47 (1H, s), 7.29-7.38 (5H, m), 7.81 (1H, dd, J = 8.4, 1.8 Hz), 7.97 (1H, d, J = 8.3 Hz), 8.37 (1H, d, J = 1.7 Hz). |
| 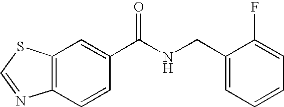 | 1H-NMR (CDCl3) δ: 4.74 (2H, d, J = 5.9 Hz), 6.66 (1H, br s), 7.06- 7.18 (2H, m), 7.25-7.32 (1H, m), 7.43-7.46 (1H, m), 7.88 (1H, dd, J = 8.5, 2.0 Hz), 8.15 (1H, d, J = 8.5 Hz), 8.48 (1H, d, J = 1.2 Hz), 9.11 (1H, s). |
| 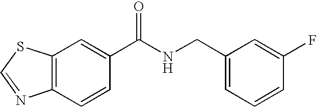 | 1H-NMR (CDCl3) δ: 4.68 (2H, d, J = 5.9 Hz), 6.65 (1H, s), 6.99 (1H, td, J = 8.5, 2.3 Hz), 7.08 (1H, d, J = 9.5 Hz), 7.15 (1H, d, J = 7.6 Hz), 7.32 (1H, td, J = 7.9, 5.9 Hz), 7.89 (1H, dd, J = 8.5, 1.5 Hz), 8.16 (1H, d, J = 8.5 Hz), 8.50 (1H, d, J = 1.5 Hz), 9.12 (1H, s). |
| 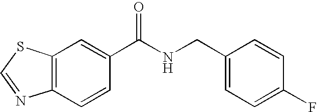 | 1H-NMR (CDCl3) δ: 4.67 (2H, d, J = 5.6 Hz), 6.48 (1H, s), 7.03-7.08 (2H, m), 7.35-7.38 (2H, m), 7.88 (1H, dd, J = 8.5, 1.7 Hz), 8.17 (1H, d, J = 8.5 Hz), 8.50 (1H, d, J = 1.7 Hz), 9.12 (1H, s). |
| 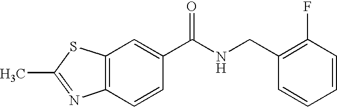 | 1H-NMR (CDCl3) δ: 2.86 (3H, s), 4.73 (2H, d, J = 5.9 Hz), 6.56 (1H, s), 7.06-7.11 (1H, m), 7.12-7.16 (1H, m), 7.26-7.32 (1H, m), 7.44 (1H, td, J = 7.6, 1.7 Hz), 7.80 (1H, dd, J = 8.5, 1.7 Hz), 7.96 (1H, d, J = 8.5 Hz), 8.34 (1H, d, J = 1.5 Hz). |
| 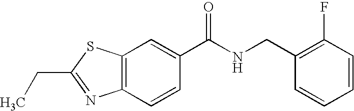 | 1H-NMR (CDCl3) δ: 1.49 (3H, t, J = 7.6 Hz), 3.17 (2H, q, J = 7.6 Hz), 4.73 (2H, d, J = 5.9 Hz), 6.57 (1H, s), 7.06-7.15 (1H, m), 7.26-7.32 (2H, m), 7.44 (1H, td, J = 7.6, 1.7 Hz), 7.80 (1H, dd, J = 8.5, 1.7 Hz), 7.97 (1H, d, J = 8.5 Hz), 8.35 (1H, d, J = 1.5 Hz). |
| 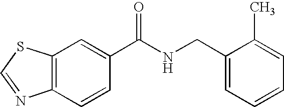 | 1H-NMR (CDCl3) δ: 2.40 (3H, s), 4.69 (2H, d, J = 5.1 Hz), 6.31 (1H, s), 7.21-7.25 (3H, m), 7.32-7.34 (1H, m), 1.87 (1H, dd, J = 8.5, 1.7 Hz), 8.16 (1H, d, J = 8.5 Hz), 8.49 (1H, d, J = 1.2 Hz), 9.11 (1H, s). |
| 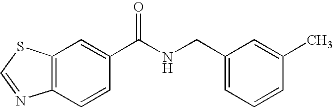 | 1H-NMR (CDCl3) δ: 2.36 (3H, s), 4.66 (2H, d, J = 5.6 Hz), 6.45 (1H, s), 7.12-7.19 (3H, m), 7.27 (1H, t, J = 7.4 Hz), 7.88 (1H, dd, J = 8.5, 1.7 Hz), 8.16 (1H, d, J = 8.5 Hz), 8.50 (1H, d, J = 1.7 Hz), 9.11 (1H, s). |
| 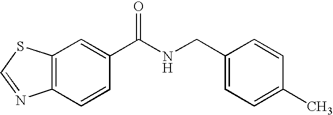 | 1H-NMR (CDCl3) δ: 2.36 (3H, s), 4.65 (2H, d, J = 5.6 Hz), 6.44 (1H, s), 7.18 (2H, d, J = 7.8 Hz), 7.28 (2H, d, J = 8.0 Hz), 1.87 (1H, dd, J = 8.5, 1.7 Hz), 8.15 (1H, d, J = 8.3 Hz), 8.49 (1H, d, J = 1.5 Hz), 9.11 (1H, s). |
| 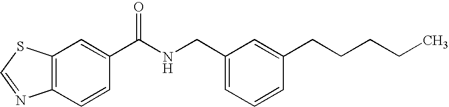 | 1H-NMR (CDCl3) δ: 0.84-0.92 (3H, m), 1.26-1.36 (4H, m), 1.56-1.64 (2H, m), 2.59 (2H, t, J = 7.6 Hz), 4.65 (2H, d, J = 5.1 Hz), 6.64 (1H, br s), 7.13- 7.30 (4H, m), 7.88 (1H, d, J = 8.0 Hz), 8.13 (1H, d, J = 8.3 Hz), 8.48 (1H, s), 9.08 (1H, s). |
| 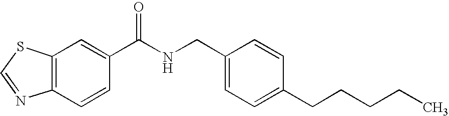 | 1H-NMR (CDCl3) δ: 0.89 (3H, t, J = 6.8 Hz), 7.29- 7.35 (4H, m), 7.55-1.64 (2H, m), 2.60 (2H, t, J = 7.7 Hz), 4.66 (2H, d, J = 5.6 Hz), 6.43 (1H, br s), 7.17- 7.31 (4H, m), 7.88 (1H, d, J = 8.5 Hz), 8.16 (1H, d, J = 8.5 Hz), 8.50 (1H, s), 9.11 (1H, s). |
| 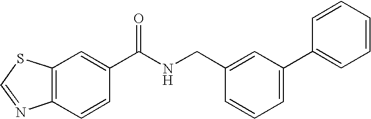 | 1H-NMR (CDCl3) δ: 4.75 (2H, d, J = 5.6 Hz), 6.60 (1H, br s), 7.33-7.38 (2H, m), 7.42-7.46 (3H, m), 7.53-7.60 (4H, m), 7.89 (1H, dd, J = 8.5, 1.7 Hz), 8.15 (1H, d, J = 8.5 Hz), 8.50 (1H, d, J = 1.7 Hz), 9.10 (1H, s). |
| 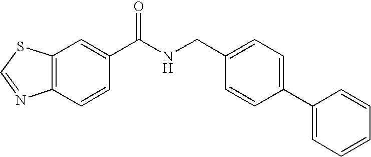 | 1H-NMR (DMSO-D6) δ: 4.57 (2H, d, J = 5.8 Hz), 7.33-7.38 (1H, m), 7.43-7.49 (4H, m), 7.63-7.66 (4H, m), 8.07 (1H, dd, J = 8.6, 1.8 Hz), 8.18 (1H, d, J = 8.5 Hz), 8.73 (1H, d, J = 1.2 Hz), 9.25 (1H, t, J = 6.0 Hz), 9.54 (1H, s). |
| 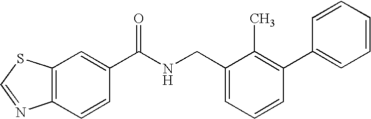 | 1H-NMR (DMSO-D6) δ: 2.22 (3H, s), 4.57 (2H, d, J = 5.4 Hz), 7.12 (1H, d, J = 7.3 Hz), 7.22-7.47 (7H, m), 8.09 (1H, d, J = 8.3 Hz), 8.18 (1H, d, J = 8.3 Hz), 8.74 (1H, s), 9.10 (1H, s), 9.54 (1H, s). |
| 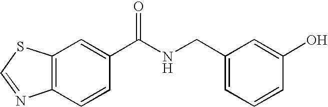 | 1H-NMR (DMSO-D6) δ: 4.45 (2H, d, J = 5.9 Hz), 6.64 (1H, dd, J = 7.2, 1.6 Hz), 6.76-6.77 (2H, m), 7.12 (1H, t, J = 8.0 Hz), 8.06 (1H, dd, J = 8.5, 1.7 Hz), 8.17 (1H, d, J = 8.5 Hz), 8.71 (1H, d, J = 1.7 Hz), 9.15 (1H, t, J = 5.9 Hz), 9.33 (1H, s), 9.54 (1H, s). |
| 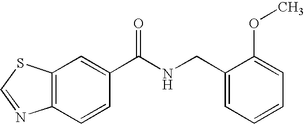 | 1H-NMR (CDCl3) δ: 3.91 (3H, s), 4.69 (2H, d, J = 5.9 Hz), 6.73 (1H, s), 6.93 (1H, d, J = 8.0 Hz), 6.96 (1H, td, J = 7.4, 1.0 Hz), 7.31 (1H, td, J = 7.9, 1.7 Hz), 7.38 (1H, dd, J = 7.4, 1.6 Hz), 7.85 (1H, dd, J = 8.5, 2.0 Hz), 8.15 (1H, d, J = 8.5 Hz), 8.47 (1H, d, J = 1.5 Hz), 9.10 (1H, s). |
| 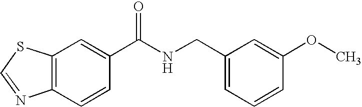 | 1H-NMR (CDCl3) δ: 3.81 (3H, s), 4.67 (2H, d, J = 5.6 Hz), 6.47 (1H, s), 6.86 (1H, dd, J = 8.2, 2.3 Hz), 6.92 (1H, s), 6.97 (1H, d, J = 7.6 Hz), 7.29 (1H, t, J = 7.9 Hz), 7.88 (1H, dd, J = 8.5, 1.7 Hz), 8.17 (1H, dd, J = 8.5 Hz), 8.50 (1H, d, J = 7.5 Hz), 9.11 (1H, s). |
| 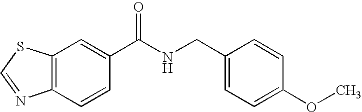 | 1H-NMR (CDCl3) δ: 3.81 (3H, s), 4.63 (2H, d, J = 5.6 Hz), 6.40 (1H, s), 6.89-6.92 (2H, m), 7.30-7.33 (2H, m), 7.87 (1H, dd, J = 8.7, 1.8 Hz), 8.16 (1H, dd, J = 8.5, 0.5 Hz), 8.49 (1H, d, J = 1.5 Hz), 9.11 (1H, s). |
| 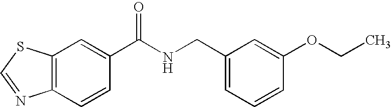 | 1H-NMR (CDCl3) δ: 1.41 (3H, t, J = 7.0 Hz), 4.03 (2H, q, J = 7.0 Hz), 4.65 (2H, d, J = 5.6 Hz), 6.53 (1H, s), 6.84 (1H, dd, J = 8.2, 2.1 Hz), 6.91-6.95 (2H, m), 7.27 (1H, t, J = 7.9 Hz), 7.88 (1H, dd, J = 8.5, 1.7 Hz), 8.15 (1H, d, J = 8.5 Hz), 8.49 (1H, d, J = 1.5 Hz), 9.11 (1H, s). |
| 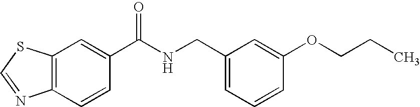 | 1H-NMR (CDCl3) δ: 1.03 (3H, t, J = 7.3 Hz), 1.76- 1.85 (2H, m), 3.92 (2H, t, J = 6.6 Hz), 4.66 (2H, d, J = 5.6 Hz), 6.46 (1H, s), 6.85 (1H, dd, J = 8.3, 2.4 Hz), 6.92-6.95 (2H, m), 7.26-7.29 (1H, m), 7.88 (1H, dd, J = 8.5, 1.7 Hz), 8.16 (1H, d, J = 8.5 Hz), 8.50 (1H, d, J = 1.2 Hz), 9.11 (1H, s). |
| 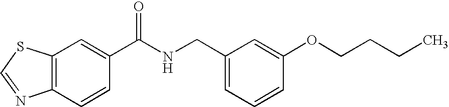 | 1H-NMR (CDCl3) δ: 0.97 (3H, t, J = 7.3 Hz), 1.44- 1.53 (2H, m), 1.72-1.79 (2H, m), 3.96 (2H, t, J = 6.5 Hz), 4.65 (2H, d, J = 5.6 Hz), 6.50 (1H, s), 6.83-6.86 (1H, m), 6.91-6.95 (2H, m), 7.27 (1H, t, J = 7.8 Hz), 7.88 (1H, dd, J = 8.7, 1.3 Hz), 8.16 (1H, d, J = 8.5 Hz), 8.50 (1H, s), 9.11 (1H, s). |
| 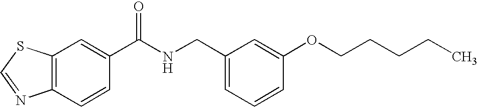 | 1H-NMR (CDCl3) δ: 0.92 (3H, t, J = 6.8 Hz), 1.33- 1.47 (4H, m), 1.74-1.81 (2H, m), 3.94 (2H, t, J = 6.6 Hz), 4.64 (2H, d, J = 5.4 Hz), 6.61 (1H, s), 6.83-6.94 (3H, m), 7.24-7.28 (1H, m), 7.88 (1H, d, J = 8.5 Hz), 8.14 (1H, d, J = 8.3 Hz), 8.49 (1H, s), 9.10 (1H, s). |
| 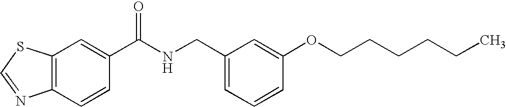 | 1H-NMR (CDCl3) δ: 0.90 (3H, t, J = 1.0 Hz), 1.31- 1.35 (4H, m), 1.41-1.49 (2H, m), 1.74-1.81 (2H, m), 3.96 (2H, t, J = 6.6 Hz), 4.66 (2H, dd, J = 5.4 Hz), 6.45 (1H, s), 6.85 (1H, dd, J = 8.2, 1.8 Hz), 6.92-6.95 (2H, m), 7.28 (1H, t, J = 7.6 Hz), 7.88 (1H, dd, J = 8.5, 2.0 Hz), 8.17 (1H, d, J = 8.5 Hz), 8.50 (1H, d, J = 1.7 Hz), 9.12 (1H, s). |
| 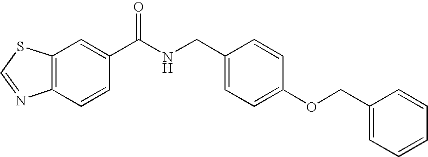 | 1H-NMR (CDCl3) δ: 4.62 (2H, d, J = 5.6 Hz), 5.07 (2H, s), 6.40-6.46 (1H, br m), 6.95-6.99 (2H, m), 7.28-7.44 (7H, m), 1.87 (1H, dd, J = 8.6, 1.7 Hz), 8.15 (1H, dd, J = 8.7, 0.5 Hz), 8.48 (1H, dd, J = 1.7, 0.5 Hz), 9.10 (1H, s). |
| 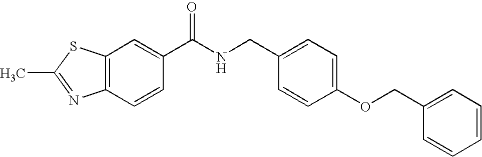 | 1H-NMR (CDCl3) δ: 2.86 (3H, s), 4.61 (2H, d, J = 5.4 Hz), 5.07 (2H, s), 6.38 (1H, s), 6.95-6.98 (2H, m), 7.26-7.44 (7H, m), 7.79 (1H, dd, J = 8.5, 1.7 Hz), 7.95 (1H, d, J = 8.5 Hz), 8.34 (1H, d, J = 1.5 Hz). |
| 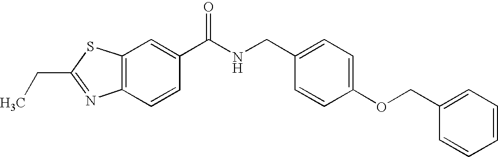 | 1H-NMR (CDCl3) δ: 1.49 (3H, t, J = 7.6 Hz), 3.17 (2H, q, J = 7.6 Hz), 4.61 (2H, d, J = 5.6 Hz), 5.07 (2H, s), 6.39 (1H, s), 6.95-6.98 (2H, m), 7.29-7.44 (7H, m), 7.79 (1H, dd, J = 8.5, 1.7 Hz), 7.96 (1H, d, J = 8.3 Hz), 8.36 (1H, d, J = 1.5 Hz). |
| 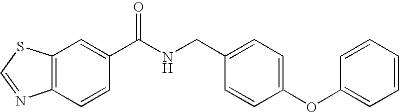 | 1H-NMR (DMSO-D6) δ: 4.51 (2H, d, J = 6.0 Hz), 6.97-7.01 (4H, m), 7.12 (1H, t, J = 7.4 Hz), 7.36-7.40 (4H, m), 8.06 (1H, dd, J = 8.5, 1.7 Hz), 8.17 (1H, d, J = 8.7 Hz), 8.71 (1H, d, J = 1.2 Hz), 9.20 (1H, t, J = 5.7 Hz), 9.54 (1H, s). |
| 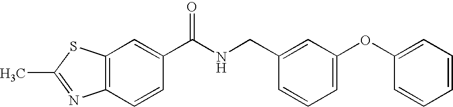 | 1H-NMR (CDCl3) δ: 2.87 (3H, s), 4.65 (2H, d, J = 5.6 Hz), 6.46 (1H, s), 6.91-6.94 (1H, m), 7.00-7.03 (3H, m), 7.09-7.14 (2H, m), 7.29-7.36 (3H, m), 7.79 (1H, dd, J = 8.5, 1.7 Hz), 7.96 (1H, d, J = 8.3 Hz), 8.33 (1H, d, J = 1.7 Hz). |
| 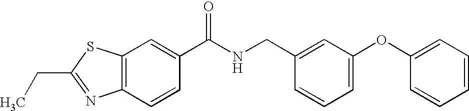 | 1H-NMR (CDCl3) δ: 1.49 (3H, t, J = 7.6 Hz), 3.15- 3.22 (2H, m), 4.66 (2H, d, J = 5.6 Hz), 6.44 (1H, s), 6.91-6.94 (1H, m), 7.01-7.03 (3H, m), 7.10-7.14 (2H, m), 7.30-7.36 (3H, m), 7.79 (1H, dd, J = 8.5, 1.7 Hz), 7.98 (1H, d, J = 8.0 Hz), 8.35 (1H, d, J = 1.5 Hz). |
| 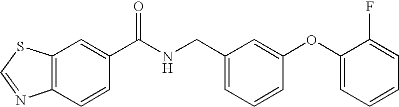 | 1H-NMR (CDCl3) δ: 4.67 (2H, d, J = 5.6 Hz), 6.50 (1H, s), 6.88 (1H, dd, J = 8.3, 2.4 Hz), 7.00 (1H, s), 7.06-7.20 (5H, m), 7.31 (1H, t, J = 7.8 Hz), 7.87 (1H, dd, J = 8.5, 1.7 Hz), 8.17 (1H, d, J = 8.5 Hz), 8.48 (1H, d, J = 1.7 Hz), 9.12 (1H, s). |
| 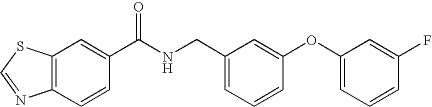 | 1H-NMR (CDCl3) δ: 4.69 (2H, d, J = 5.9 Hz), 6.51 (1H, s), 6.70 (1H, dt, J = 10.2, 2.3 Hz), 6.77-6.83 (2H, m), 6.95-6.98 (1H, m), 7.05-7.06 (1H, m), 7.17 (1H, d, J = 7.6 Hz), 7.24-7.30 (1H, m), 7.35 (1H, t, J = 7.8 Hz), 7.88 (1H, dd, J = 8.4, 1.8 Hz), 8.17 (1H, d, J = 8.5 Hz), 8.49 (1H, d, J = 1.7 Hz), 9.12 (1H, s). |
| 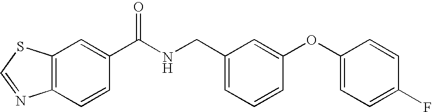 | 1H-NMR (CDCl3) δ: 4.67 (2H, d, J = 5.9 Hz), 6.51 (1H, s), 6.88 (1H, dd, J = 8.2, 2.6 Hz), 6.97-7.06 (5H, m), 7.10 (1H, d, J = 7.6 Hz), 7.31 (1H, t, J = 7.9 Hz), 7.87 (1H, dd, J = 8.5, 1.7 Hz), 8.17 (1H, d, J = 8.5 Hz), 8.49 (1H, d, J = 1.7 Hz), 9.12 (1H, s). |
| 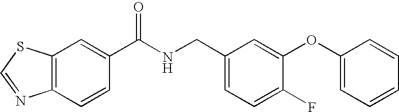 | 1H-NMR (DMSO-D6) δ: 4.48 (2H, d, J = 5.9 Hz), 6.97 (2H, d, J = 7.8 Hz), 7.09-7.13 (1H, m), 7.16 (1H, dd, J = 8.0, 2.2 Hz), 7.19-7.23 (1H, m), 7.33- 7.39 (3H, m), 8.00 (1H, dd, J = 8.5, 1.7 Hz), 8.15 (1H, d, J = 8.5 Hz), 8.65 (1H, d, J = 1.7 Hz), 9.18 (1H, t, J = 5.9 Hz), 9.54 (1H, s). |
| 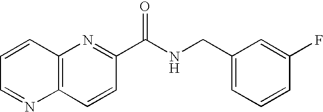 | 1H-NMR (CDCl3) δ: 4.75 (2H, d, J = 6.3 Hz), 6.99 (1H, t, J = 8.3 Hz), 7.12 (1H, d, J = 8.2 Hz), 7.19 (1H, d, J = 7.5 Hz), 7.29-7.36 (1H, m), 7.70 (1H, dd, J = 8.6, 4.2 Hz), 8.39 (1H, d, J = 8.7 Hz), 8.48 (1H, brs), 8.56 (1H, br s), 8.58 (2H, s), 9.06 (1H, dd, J = 4.0, 1.8 Hz). |
| 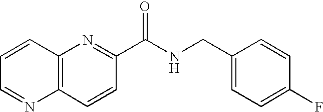 | 1H-NMR (CDCl3) δ: 4.72 (2H, d, J = 6.0 Hz), 7.06 (1H, t, J = 8.6 Hz), 7.39 (1H, dd, J = 8.4, 5.4 Hz), 7.70 (1H, dd, J = 8.6, 4.2 Hz), 8.39 (1H, dd, J = 8.4, 1.7 Hz), 8.51 (1H, brs), 8.58 (2H, dd, J = 9.9, 8.9 Hz), 9.06 (1H, dd, J = 4.4, 1.7 Hz). |
| 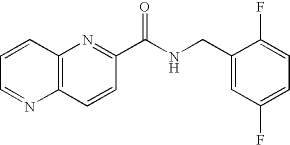 | 1H-NMR (CDCl3) δ: 4.77 (2H, d, J = 6.0 Hz), 6.93- 7.00 (1H, m), 7.02-7.08 (1H, m), 7.15-7.20 (1H, m), 7.71 (1H, dd, J = 8.7, 4.1 Hz), 8.42 (1H, dd, J = 8.2, 1.7 Hz), 8.58 (1H, brs), 8.57 (2H, s), 9.07 (1H, dd, J = 4.1, 1.7 Hz). |
| 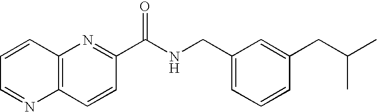 | 1H-NMR (CDCl3) δ: 0.91 (6H, d, J = 6.6 Hz), 1.82- 1.92 (1H, m), 2.49 (2H, d, J = 7.1 Hz), 4.73 (2H, d, J = 6.1 Hz), 7.10 (1H, d, J = 7.3 Hz), 7.19 (1H, s), 7.24 (1H, d, J = 7.6 Hz), 7.29 (1H, t, J = 7.4 Hz), 7.69 (1H, dd, J = 8.5, 4.1 Hz), 8.39 (1H, dd, J = 8.4, 1.7 Hz), 8.51 (1H, s), 8.58 (2H, d, J = 9.9, 8.9 Hz), 9.05 (1H, dd, J = 4.1, 1.7 Hz). |
| 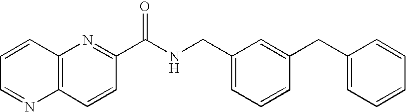 | 1H-NMR (CDCl3) δ: 3.99 (2H, s), 4.71 (2H, d, J = 6.0 Hz), 7.13-7.16 (1H, m), 7.16-7.21 (3H, m), 7.25- 7.31 (5H, m), 7.69 (1H, dd, J = 8.7, 4.1 Hz), 8.37 (1H, dd, J = 8.6, 1.1 Hz), 8.47 (1H, br s), 8.55-8.60 (2H, m), 9.06 (1H, dd, J = 4.1, 1.7 Hz). |
| 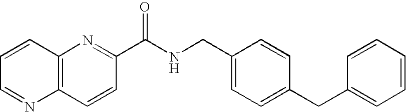 | 1H-NMR (CDCl3) δ: 3.98 (2H, s), 4.71 (2H, d, J = 6.0 Hz), 7.18-7.35 (9H, m), 7.69 (1H, dd, J = 8.6, 4.2 Hz), 8.38 (1H, dd, J = 8.4, 1.7 Hz), 8.46 (1H, brs), 8.57 (2H, dd, J = 10.6, 8.9 Hz), 9.04 (1H, dd, J = 4.0, 1.7 Hz). |
| 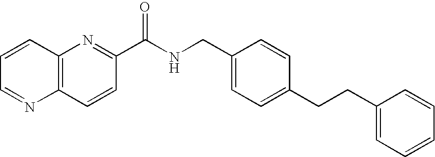 | 1H-NMR (CDCl3) δ: 2.92 (4H, s), 4.72 (2H, d, J = 6.0 Hz), 7.18-7.35 (9H, m), 7.69 (1H, dd, J = 8.6, 4.2 Hz), 8.38 (1H, d, J = 8.4 Hz), 8.48 (1H, brs), 8.58 (2H, dd, J = 13.1, 9.1 Hz), 9.05 (1H, d, J = 4.0 Hz). |
| 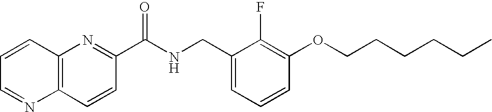 | 1H-NMR (CDCl3) δ: 0.89-0.92 (3H, m), 1.34-1.59 (6H, m), 1.80-1.85 (2H, m), 4.03 (2H, t, J = 6.4 Hz), 4.79 (2H, d, J = 5.6 Hz), 6.90-6.95 (1H, m), 7.01- 7.05 (2H, m), 7.70 (1H, dd, J = 8.2, 3.9 Hz), 8.41 (1H, d, J = 8.7 Hz), 8.52-8.57 (3H, m), 9.05 (1H, d, J = 2.2 Hz). |
| 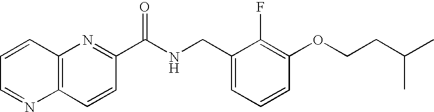 | 1H-NMR (CDCl3) δ: 0.97 (6H, d, J = 6.8 Hz), 1.70- 1.75 (2H, m), 1.81-1.91 (1H, m), 4.06 (2H, t, J = 6.6 Hz), 4.79 (2H, d, J = 6.3 Hz), 6.90-6.96 (1H, m), 7.00-7.06 (2H, m), 7.70 (1H, dd, J = 8.5, 4.1 Hz), 8.41 (1H, dd, J = 8.6, 1.6 Hz), 8.51-8.58 (3H, m), 9.05 (1H, dd, J = 4.1, 1.4 Hz). |
| 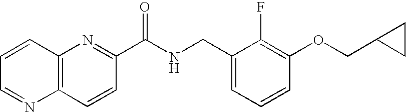 | 1H-NMR (CDCl3) δ: 0.36-0.39 (2H, m), 0.63-0.69 (2H, m), 1.28-1.34 (1H, m), 3.88 (2H, d, J = 7.0 Hz), 4.79 (2H, d, J = 6.5 Hz), 6.89-6.93 (1H, m), 7.01-7.05 (2H, m), 7.70 (1H, dd, J = 8.6, 4.2 Hz), 8.41 (1H, dd, J = 8.7, 1.7 Hz), 8.52-8.57 (3H, m), 9.05 (1H, t, J = 2.1 Hz). |
| 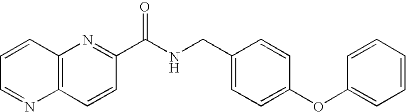 | 1H-NMR (CDCl3) δ: 4.72 (2H, d, J = 6.0 Hz), 7.01 (4H, d, J = 8.7 Hz), 7.11 (2H, t, J = 7.4 Hz), 7.34 (2H, t, J = 8.1 Hz), 7.39 (1H, d, J = 8.7 Hz), 7.70 (1H, dd, J = 8.6, 4.2 Hz), 8.39 (1H, d, J = 8.7 Hz), 8.50 (1H, brs), 8.58 (2H, dd, J = 12.4, 8.7 Hz), 9.06 (1H, d, J = 4.0 Hz). |
| 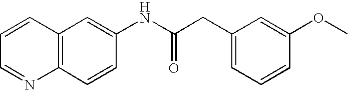 | 1H-NMR (CDCl3) δ: 3.78 (2H, s), 3.83 (3H, s), 6.88- 6.97 (3H, m), 7.32-7.43 (3H, m), 7.51 (1H, br s), 7.98 (1H, d, J = 8.9 Hz), 8.09 (1H, d, J = 8.0 Hz), 8.32 (1H, d, J = 2.2 Hz), 8.82 (1H, dd, J = 4.2, 1.6 Hz). |
| 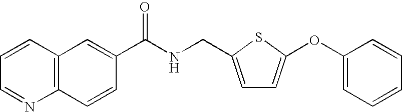 | 1H-NMR (CDCl3) δ: 5.12 (2H, d, J = 5.6 Hz), 6.43 (1H, d, J = 3.9 Hz), 6.88 (1H, d, J = 3.6 Hz), 7.09- 7.15 (3H, m), 7.32-7.36 (2H, m), 7.45 (1H, dd, J = 8.3, 4.2 Hz), 8.02 (1H, s), 8.06-8.07 (2H, m), 8.19- 8.22 (2H, m), 8.95 (1H, dd, J = 4.2, 1.8 Hz). |
| 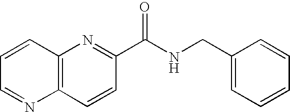 | 1H-NMR (CDCl3) δ: 4.75 (2H, d, J = 6.0 Hz), 7.30- 7.34 (1H, m), 7.36-7.44 (4H, m), 7.69 (1H, dd, J = 8.6, 4.2 Hz), 8.38 (1H, dd, J = 8.4, 1.3 Hz), 8.56 (1H, br s), 8.58 (2H, dd, J = 13.3, 8.9 Hz), 9.05 (1H, dd, J = 4.4, 1.7 Hz). |
| 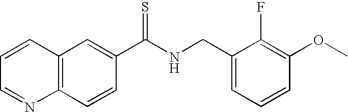 | 1H-NMR (CDCl3) δ: 3.92 (3H, s), 5.14 (2H, d, J = 5.6 Hz), 6.96-7.01 (1H, m), 7.06-7.14 (2H, m), 7.46 (1H, dd, J = 8.5, 4.3 Hz), 7.98 (1H, br s), 8.09-8.10 (2H, m), 8.21-8.24 (2H, m), 8.97 (1H, dd, J = 4.1, 1.7 Hz). |
| 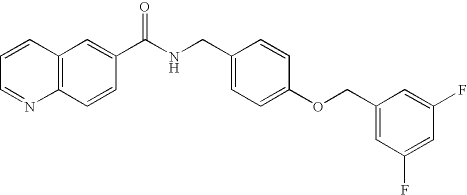 | 1H-NMR (CDCl3) δ: 4.65 (2H, d, J = 4.8 Hz), 5.05 (2H, s), 6.56 (1H, br s), 6.74-6.78 (1H, m), 6.92-6.98 (3H, m), 7.25-7.35 (3H, m), 7.46-7.50 (1H, m), 8.05 (1H, d, J = 8.5 Hz), 8.15 (1H, d, J = 8.5 Hz), 8.24 (1H, d, J = 7.5 Hz), 8.33 (1H, s), 8.98 (1H, s). |
| 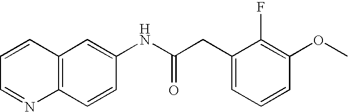 | 1H-NMR (CDCl3) δ: 3.82 (2H, s), 3.91 (3H, s), 6.94- 6.99 (2H, m), 7.08-7.15 (1H, m), 7.37 (1H, dd, J = 8.3, 4.2 Hz), 7.48 (1H, dd, J = 8.9, 2.4 Hz), 7.62 (1H, br s), 8.00 (1H, d, J = 8.9 Hz), 8.09 (1H, d, J = 8.2 Hz), 8.33 (1H, d, J = 1.9 Hz), 8.82 (1H, dd, J = 4.1, 1.4 Hz). |
| 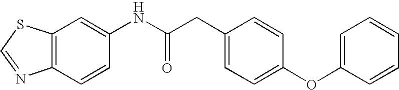 | 1H-NMR (CDCl3) δ: 3.76 (2H, s), 7.01-7.07 (4H, m), 7.12-7.41 (7H, m), 8.00 (1H, d, J = 8.9 Hz), 8.52 (1H, d, J = 1.7 Hz), 8.91 (1H, s). |
| 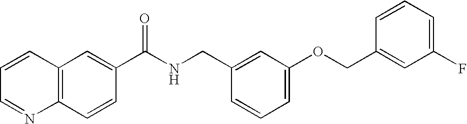 | 1H-NMR (CDCl3) δ: 4.69 (2H, d, J = 5.6 Hz), 5.07 (2H, s), 6.57 (1H, br s), 6.91-7.33 (8H, m), 7.48 (1H, dd, J = 8.2, 4.3 Hz), 8.05 (1H, dd, J = 8.9, 1.9 Hz), 8.16 (1H, d, J = 8.7 Hz), 8.24 (1H, d, J = 7.2 Hz), 8.33 (1H, d, J = 1.7 Hz), 8.99 (1H, dd, J = 4.2, 1.6 Hz). |
| 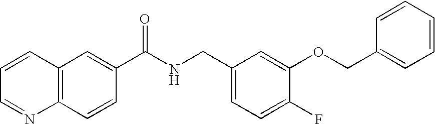 | 1H-NMR (CDCl3) δ: 4.63 (2H, d, J = 5.8 Hz), 5.15 (2H, s), 6.52 (1H, br s), 6.90-6.94 (1H, m), 7.04-7.10 (2H, m), 7.28-7.35 (3H, m), 7.42 (2H, d, J = 7.2 Hz), 7.49 (1H, dd, J = 8.3, 4.2 Hz), 8.02 (1H, dd, J = 8.8, 2.1 Hz), 8.17 (1H, d, J = 8.7 Hz), 8.25 (1H, d, J = 8.2 Hz), 8.31 (1H, d, J = 1.9 Hz), 9.00 (1H, dd, J = 4.1, 1.7 Hz). |
| 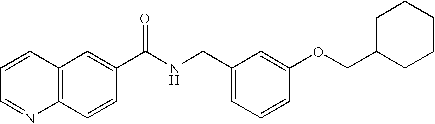 | 1H-NMR (CDCl3) δ: 1.00-1.08 (2H, m), 1.17-1.34 (3H, m), 1.60-1.89 (6H, m), 3.76 (2H, d, J = 6.3 Hz), 4.68 (2H, d, J = 5.6 Hz), 6.55 (1H, br s), 6.85 (1H, dd, J = 8.3, 2.2 Hz), 6.92-6.97 (2H, m), 7.26-7.30 (1H, m), 7.48 (1H, dd, J = 8.3, 4.1 Hz), 8.07 (1H, dd, J = 8.8, 2.0 Hz), 8.16 (1H, d, J = 8.8 Hz), 8.24 (1H, d, J = 8.3 Hz), 8.33 (1H, d, J = 1.7 Hz), 8.99 (1H, dd, J = 4.3, 1.6 Hz). |
| 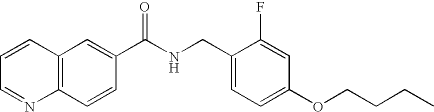 | 1H-NMR (CDCl3) δ: 0.97 (3H, t, J = 7.4 Hz), 1.43- 1.53 (2H, m), 1.72-1.80 (2H, m), 3.94 (2H, t, J = 6.4 Hz), 4.68 (2H, d, J = 5.6 Hz), 6.60-6.70 (3H, m), 7.35 (1H, t, J = 8.5 Hz), 7.47 (1H, dd, J = 8.2, 4.1 Hz), 8.05 (1H, d, J = 8.7 Hz), 8.15 (1H, d, J = 8.7 Hz), 8.24 (1H, d, J = 8.0 Hz), 8.30-8.31 (1H, m), 8.98 (1H, d, J = 2.7 Hz). |
| 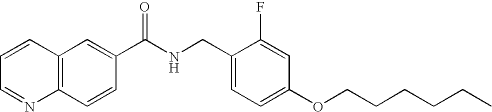 | 1H-NMR (CDCl3) δ: 0.90 (3H, t, J = 6.7 Hz), 1.31- 1.35 (4H, m), 1.41-1.48 (2H, m), 1.73-1.80 (2H, m), 3.93 (2H, t, J = 6.6 Hz), 4.68 (2H, d, J = 5.9 Hz), 6.58 (1H, br s), 6.63-6.69 (2H, m), 7.35 (1H, t, J = 8.5 Hz), 7.47 (1H, dd, J = 8.3, 4.1 Hz), 8.04 (1H, dd, J = 8.8, 1.7 Hz), 8.15 (1H, d, J = 8.8 Hz), 8.23 (1H, d, J = 8.3 Hz), 8.30 (1H, d, J = 1.5 Hz), 8.98 (1H, t, J = 2.1 Hz). |
| 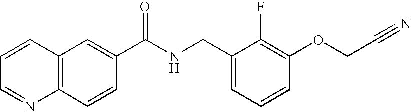 | 1H-NMR (CDCl3) δ: 4.78 (2H, d, J = 5.1 Hz), 4.85 (2H, s), 6.79 (1H, br s), 7.05-7.15 (2H, m), 7.21-7.27 (1H, m), 7.47 (1H, dd, J = 7.8, 4.0 Hz), 8.07 (1H, d, J = 8.0 Hz), 8.15 (1H, d, J = 8.2 Hz), 8.23 (1H, d, J = 8.0 Hz), 8.33 (1H, s), 8.98-8.99 (1H, m). |
| 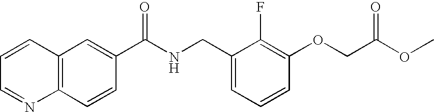 | 1H-NMR (CDCl3) δ: 3.81 (3H, s), 4.72 (2H, s), 4.76 (2H, d, J = 3.6 Hz), 6.77 (1H, br s), 6.84-6.89 (1H, m), 7.02-7.14 (2H, m), 7.44-7.50 (1H, m), 8.08 (1H, s), 8.14 (1H, s), 8.24 (1H, d, J = 7.0 Hz), 8.32 (1H, s), 8.98 (1H, s). |
| 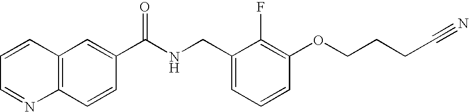 | 1H-NMR (CDCl3) δ: 2.15-2.21 (2H, m), 2.65 (2H, t, J = 6.9 Hz), 4.16 (2H, t, J = 5.5 Hz), 4.76 (2H, d, J = 5.7 Hz), 6.71 (1H, br s), 6.91-6.96 (1H, m), 7.06- 7.08 (2H, m), 7.47 (1H, dd, J = 8.1, 4.0 Hz), 8.07 (1H, d, J = 8.7 Hz), 8.15 (1H, d, J = 8.4 Hz), 8.24 (1H, d, J = 8.4 Hz), 8.33 (1H, s), 8.99 (1H, d, J = 2.4 Hz). |
| 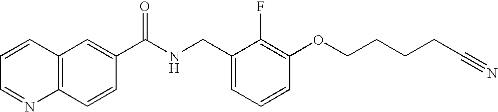 | 1H-NMR (CDCl3) δ: 1.91-2.03 (4H, m), 2.49 (2H, t, J = 6.7 Hz), 4.09 (2H, t, J = 5.7 Hz), 4.76 (2H, d, J = 5.7 Hz), 6.69 (1H, br s), 6.90-6.94 (1H, m), 7.05- 7.07 (2H, m), 7.48 (1H, dd, J = 8.4, 4.4 Hz), 8.06 (1H, dd, J = 8.7, 2.0 Hz), 8.16 (1H, d, J = 9.1 Hz), 8.24 (1H, d, J = 7.7 Hz), 8.32 (1H, d, J = 1.7 Hz), 8.99 (1H, dd, J = 4.2, 1.5 Hz). |
| 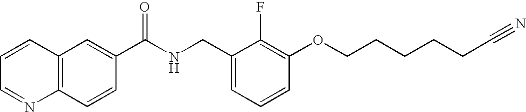 | 1H-NMR (CDCl3) δ: 1.65-1.91 (6H, m), 2.40 (2H, t, J = 6.9 Hz), 4.06 (2H, t, J = 6.0 Hz), 4.76 (2H, d, J = 5.7 Hz), 6.74 (1H, br s), 6.89-6.94 (1H, m), 7.02- 7.07 (2H, m), 7.47 (1H, dd, J = 8.2, 4.2 Hz), 8.06 (1H, dd, J = 8.7, 2.0 Hz), 8.15 (1H, d, J = 8.7 Hz), 8.24 (1H, d, J = 7.7 Hz), 8.32 (1H, d, J = 1.7 Hz), 8.98 (1H, dd, J = 4.0, 1.7 Hz). |
| 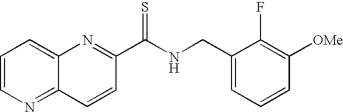 | 1H-NMR (CDCl3) δ: 3.92 (3H, s), 5.20 (2H, d, J = 5.6 Hz), 6.95-7.00 (1H, m), 7.07-7.10 (2H, m), 7.69 (1H, dd, J = 8.4, 4.3 Hz), 8.40 (1H, d, J = 8.5 Hz), 8.51 (1H, d, J = 9.0 Hz), 9.04 (1H, dd, J = 4.1, 1.5 Hz), 9.09 (1H, d, J = 8.8 Hz), 10.50 (1H, s). |
| 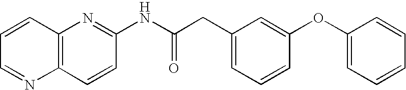 | 1H-NMR (CDCl3) δ: 3.80 (2H, s), 6.96-7.14 (5H, m), 7.31-7.39 (4H, m), 7.58 (1H, dd, J = 8.6, 4.2 Hz), 8.10 (1H, d, J = 8.5 Hz), 8.37 (1H, br s), 8.40 (1H, d, J = 9.2 Hz), 8.66 (1H, d, J = 9.4 Hz), 8.87 (1H, dd, J = 4.2, 1.6 Hz). |
| 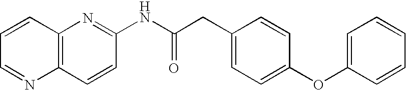 | 1H-NMR (CDCl3) δ: 3.81 (2H, s), 6.94-7.14 (5H, m), 7.32-7.37 (4H, m), 7.57 (1H, dd, J = 8.6, 4.2 Hz), 8.09 (1H, dq, J = 8.5, 0.8 Hz), 8.37 (1H, br s), 8.40 (1H, d, J = 9.2 Hz), 8.67 (1H, d, J = 9.4 Hz), 8.87 (1H, dd, J = 4.3, 1.7 Hz). |
| 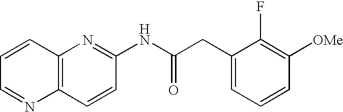 | 1H-NMR (CDCl3) δ: 3.86 (2H, s), 3.91 (3H, s), 6.94- 7.00 (2H, m), 7.09-7.15 (1H, m), 7.57 (1H, dd, J = 8.6, 4.2 Hz), 8.09 (1H, dd, J = 8.6, 0.7 Hz), 8.23 (1H, br s), 8.38 (1H, d, J = 9.2 Hz), 8.63 (1H, d, J = 9.2 Hz), 8.87 (1H, d, J = 4.2 Hz). |
| 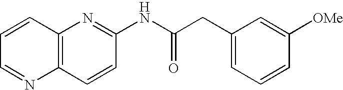 | 1H-NMR (CDCl3) δ: 3.80 (2H, s), 3.84 (3H, s), 6.89- 6.92 (2H, m), 6.94-6.98 (1H, m), 7.32-7.38 (1H, m), 7.56 (1H, dd, J = 8.6, 4.2 Hz), 8.05-8.09 (1H, m), 8.10 (1H, br s), 8.38 (1H, d, J = 9.3 Hz), 8.66 (1H, d, J = 9.3 Hz), 8.86 (1H, dd, J = 4.2, 1.6 Hz). |
| 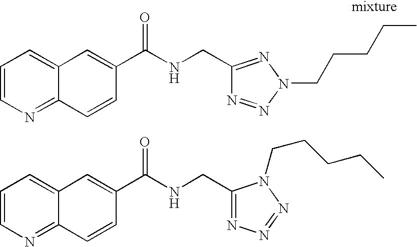 | 1H-NMR (CDCl3) δ: 0.87-0.93 (3.0H, m), 1.30-1.41 (4.0H, m), 1.94-2.06 (2.0H, m), 4.53 (0.6H, t, J = 7.4 Hz), 4.61 (1.4H, t, J = 7.2 Hz), 4.98 (0.7H, d, J = 5.9 Hz), 5.01 (1.4H, d, J = 5.4 Hz), 7.10 (0.7H, s), 7.48 (1.0H, dd, J = 8.3, 4.1 Hz), 7.66 (0.3H, s), 8.08-8.26 (3.0H, m), 8.36 (0.3H, d, J = 2.0 Hz), 8.38 (0.6H, d, J = 1.7 Hz), 8.99-9.01 (1H, m). |
| 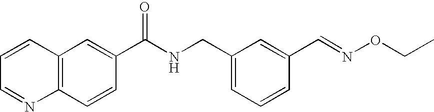 | 1H-NMR (CDCl3) δ: 1.32 (3H, t, J = 7.1 Hz), 4.23 (2H, q, J = 7.1 Hz), 4.73 (2H, d, J = 5.6 Hz), 6.60 (1H, s), 7.36-7.42 (3H, m), 7.48 (1H, dd, J = 8.3, 4.4 Hz), 7.51 (1H, dt, J = 7.1, 1.6 Hz), 7.64 (1H, s), 8.05- 8.08 (1H, m), 8.16 (1H, d, J = 8.8 Hz), 8.24 (1H, dd, J = 8.5, 1.0 Hz), 8.34 (1H, d, J = 2.0 Hz), 8.99 (1H, dd, J = 4.3, 1.8 Hz). |
| 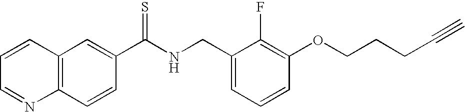 | 1H-NMR (CDCl3) δ: 1.98 (1H, t, J = 2.7 Hz), 2.02- 2.08 (2H, m), 2.45 (2H, td, J = 7.0, 2.6 Hz), 4.16 (2H, t, J = 6.1 Hz), 5.13 (2H, d, J = 5.4 Hz), 6.97-7.09 (3H, m), 7.45 (1H, dd, J = 8.4, 4.3 Hz), 8.02 (1H, s), 8.08-8.08 (2H, m), 8.20-8.22 (2H, m), 8.95 (1H, dd, J = 4.1, 1.7 Hz). |
| 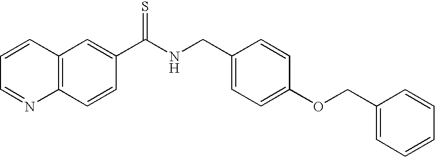 | 1H-NMR (CDCl3) δ: 4.97 (2H, d, J = 4.9 Hz), 5.09 (2H, s), 7.01 (2H, d, J = 8.5 Hz), 7.31-7.48 (8H, m), 7.84 (1H, s), 8.07-8.15 (2H, m), 8.22-8.24 (2H, m), 8.98 (1H, d, J = 3.2 Hz). |
| 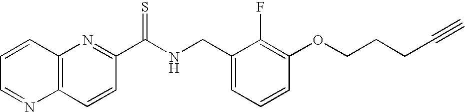 | 1H-NMR (CDCl3) δ: 1.98 (1H, t, J = 2.7 Hz), 2.02- 2.09 (2H, m), 2.46 (2H, td, J = 6.9, 2.6 Hz), 4.17 (2H, t, J = 6.1 Hz), 5.19 (2H, d, J = 5.9 Hz), 6.96- 7.01 (1H, m), 7.06-7.08 (2H, m), 7.69 (1H, dd, J = 8.7, 4.3 Hz), 8.39-8.42 (1H, m), 8.51 (1H, d, J = 9.0 Hz), 9.05 (1H, dd, J = 4.1, 1.5 Hz), 9.09 (1H, d, J = 8.8 Hz), 10.49 (1H, s). |
| 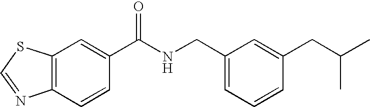 | 1H-NMR (CDCl3) δ: 9.09 (1H, s), 8.49 (1H, t, J = 0.9 Hz), 8.14 (1H, d, J = 8.5 Hz), 7.88 (1H, dt, J = 8.5, 0.9 Hz), 7.27 (1H, t, J = 7.6 Hz), 7.19 (1H, d, J = 7.6 Hz), 7.15 (1H, s), 7.09 (1H, d, J = 7.6 Hz), 6.61 (1H, s), 4.66 (2H, d, J = 5.6 Hz), 2.47 (2H, d, J = 7.3 Hz), 1.91-1.81 (1H, m), 0.90 (6H, d, J = 6.6 Hz). |
| 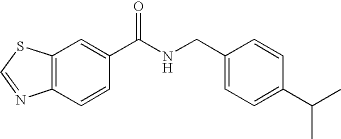 | 1H-NMR (CDCl3) δ: 9.11 (1H, s), 8.50 (1H, d, J = 1.7 Hz), 8.16 (1H, d, J = 8.5 Hz), 7.87 (1H, dd, J = 8.5, 1.7 Hz), 7.32 (2H, d, J = 8.2 Hz), 7.27-7.23 (2H, m), 6.45 (1H, s), 4.66 (2H, d, J = 5.6 Hz), 2.95-2.88 (1H, m), 1.25 (6H, d, J = 7.0 Hz). |
| 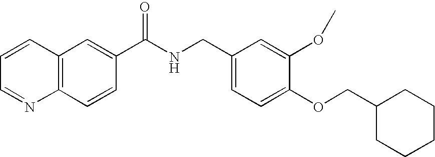 | 1H-NMR (CDCl3) δ: 0.98-1.08 (2H, m), 1.18-1.34 (3H, m), 1.67-1.78 (3H, m), 1.84-1.92 (3H, m), 3.80 (2H, d, J = 6.3 Hz), 3.86 (3H, s), 4.64 (2H, d, J = 5.6 Hz), 6.52 (1H, s), 6.84-6.92 (3H, m), 7.47 (1H, dd, J = 8.3, 4.1 Hz), 8.06 (1H, dd, J = 8.8, 2.2 Hz), 8.15 (1H, d, J = 8.8 Hz), 8.23 (1H, dd, J = 8.3, 1.0 Hz), 8.33 (1H, d, J = 2.0 Hz), 8.99 (1H, dd, J = 4.4, 1.7 Hz). |
| 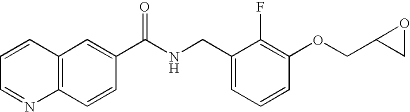 | 1H-NMR (CDCl3) δ: 2.78 (1H, dd, J = 4.9, 2.7 Hz), 2.92 (1H, dd, J = 4.8, 4.3 Hz), 3.37-3.41 (1H, m), 4.03 (1H, dd, J = 11.2, 5.6 Hz), 4.32 (1H, dd, J = 11.3, 3.0 Hz), 4.76 (2H, dd, J = 5.9, 1.0 Hz), 6.74 (1H, s), 6.94-6.98 (1H, m), 7.03-7.09 (2H, m), 7.47 (1H, dd, J = 8.3, 4.4 Hz), 8.06 (1H, dd, J = 8.9, 2.1 Hz), 8.15 (1H, d, J = 9.0 Hz), 8.23 (1H, dd, J = 8.4, 1.1 Hz), 8.31 (1H, d, J = 2.0 Hz), 8.98 (1H, dd, J = 4.1, 1.7 Hz). |
| 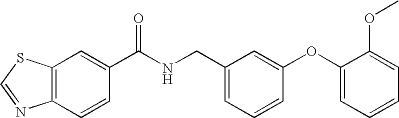 | 1H-NMR (CDCl3) δ: 9.12 (1H, s), 8.47 (1H, d, J = 1.7 Hz), 8.16 (1H, d, J = 8.7 Hz), 7.86 (1H, dd, J = 8.5, 1.7 Hz), 7.27 (1H, t, J = 7.8 Hz), 7.17-7.13 (1H, m), 7.05 (1H, d, J = 7.7 Hz), 7.00-6.98 (3H, m), 6.95- 6.91 (1H, m), 6.84 (1H, dd, J = 7.8, 2.3 Hz), 6.50 (1H, s), 4.65 (2H, d, J = 5.6 Hz), 3.82 (3H, s). |
| 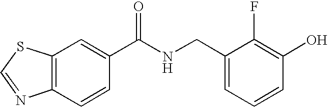 | 1H-NMR (DMSO-D6) δ: 9.78 (1H, s), 9.54 (1H, s), 9.13 (1H, t, J = 5.7 Hz), 8.70 (1H, d, J = 1.7 Hz), 8.16 (1H, d, J = 8.5 Hz), 8.05 (1H, dd, J = 8.5, 1.7 Hz), 7.95 (1H, s), 6.93 (1H, t, J = 7.8 Hz), 6.87-6.77 (2H, m), 4.53 (2H, d, J = 5.6 Hz). |
| 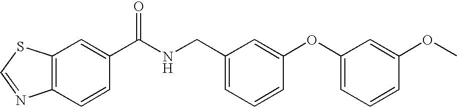 | 1H-NMR (CDCl3) δ: 9.11 (1H, s), 8.48 (1H, s), 8.15 (1H, d, J = 8.5 Hz), 7.87 (1H, dd, J = 8.6, 1.6 Hz), 7.32 (1H, t, J = 7.8 Hz), 7.22 (1H, t, J = 8.1 Hz), 7.11 (1H, d, J = 7.5 Hz), 7.03 (1H, s), 6.94 (1H, dd, J = 8.1, 2.3 Hz), 6.67-6.65 (1H, m), 6.60-6.57 (3H, m), 4.66 (2H, d, J = 5.8 Hz), 3.77 (3H, s). |
| 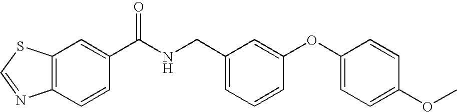 | 1H-NMR (CDCl3) δ: 9.11 (1H, s), 8.48 (1H, d, J = 1.7 Hz), 8.15 (1H, d, J = 8.5 Hz), 7.87 (1H, dd, J = 8.5, 1.7 Hz), 7.28 (1H, t, J = 7.8 Hz), 7.05 (1H, d, J = 7.7 Hz), 7.00-6.95 (3H, m), 6.90-6.84 (3H, m), 6.59 (1H, s), 4.64 (2H, d, J = 5.6 Hz), 3.79 (3H, s). |
| 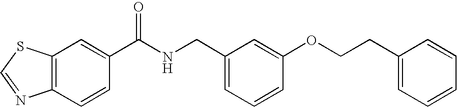 | 1H-NMR (CDCl3) δ: 9.09 (1H, s), 8.48 (1H, d, J = 1.2 Hz), 8.14 (1H, d, J = 8.7 Hz), 7.87 (1H, dd, J = 8.7, 1.7 Hz), 7.33-7.20 (6H, m), 6.94 (1H, d, J = 7.5 Hz), 6.91 (1H, s), 6.83 (1H, dd, J = 8.3, 2.1 Hz), 6.57 (1H, s), 4.63 (2H, d, J = 5.6 Hz), 4.17 (2H, t, J = 7.1 Hz), 3.09 (2H, t, J = 7.0 Hz). |
| 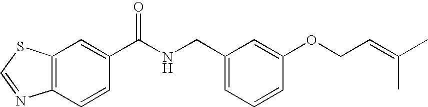 | 1H-NMR (CDCl3) δ: 9.12 (1H, s), 8.50 (1H, d, J = 1.7 Hz), 8.17 (1H, d, J = 8.5 Hz), 7.89 (1H, dd, J = 8.6, 1.8 Hz), 7.28 (1H, t, J = 7.8 Hz), 6.96-6.93 (2H, m), 6.87 (1H, dd, J = 8.0, 2.2 Hz), 6.49 (1H, s), 5.50- 5.46 (1H, m), 4.67 (2H, d, J = 5.8 Hz), 4.51 (2H, d, J = 7.0 Hz), 1.78 (3H, d, J = 0.7 Hz), 1.73 (3H, s). |
| 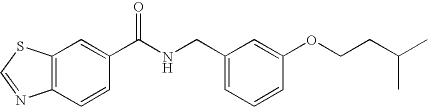 | 1H-NMR (CDCl3) δ: 9.11 (1H, s), 8.50 (1H, d, J = 1.7 Hz), 8.16 (1H, d, J = 8.5 Hz), 7.88 (1H, dd, J = 8.5, 1.7 Hz), 7.27 (1H, t, J = 7.9 Hz), 6.95-6.91 (2H, m), 6.84 (1H, dd, J = 7.8, 2.2 Hz), 6.50 (1H, s), 4.66 (2H, d, J = 5.4 Hz), 3.99 (2H, t, J = 6.7 Hz), 1.88- 1.78 (1H, m), 1.67 (2H, q, J = 6.7 Hz), 0.95 (6H, d, J = 6.6 Hz). |
| 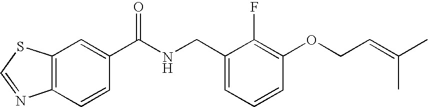 | 1H-NMR (CDCl3) δ: 9.11 (1H, s), 8.48 (1H, d, J = 7.4 Hz), 8.16 (1H, d, J = 8.5 Hz), 7.88 (1H, dd, J = 8.6, 1.8 Hz), 7.07-6.99 (2H, m), 6.94 (1H, td, J = 7.8, 2.1 Hz), 6.60 (1H, s), 5.53-5.48 (1H, m), 4.74 (2H, d, J = 5.3 Hz), 4.59 (2H, d, J = 6.8 Hz), 1.79 (3H, s), 7.75 (3H, s). |
| 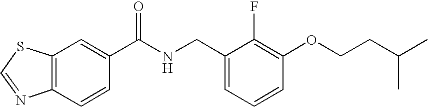 | 1H-NMR (CDCl3) δ: 9.11 (1H, s), 8.48 (1H, d, J = 1.4 Hz), 8.16 (1H, d, J = 8.7 Hz), 7.88 (1H, dd, J = 8.5, 1.7 Hz), 7.06-6.98 (2H, m), 6.93 (1H, td, J = 7.9, 1.8 Hz), 6.59 (1H, s), 4.74 (2H, d, J = 5.8 Hz), 4.06 (2H, t, J = 6.6 Hz), 1.91-1.81 (1H, m), 1.72 (2H, q, J = 6.8 Hz), 0.97 (6H, d, J = 6.8 Hz). |
| 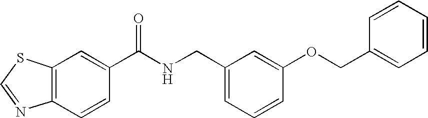 | 1H-NMR (CDCl3) δ: 9.11 (1H, s), 8.48 (1H, d, J = 1.7 Hz), 8.16 (1H, d, J = 8.5 Hz), 7.87 (1H, dd, J = 8.7, 1.7 Hz), 7.43-7.26 (6H, m), 7.00-6.96 (2H, m), 6.93 (1H, dd, J = 8.2, 2.2 Hz), 6.47 (1H, s), 5.07 (2H, s), 4.66 (2H, d, J = 5.6 Hz). |
| 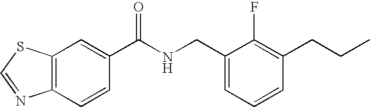 | 1H-NMR (CDCl3) δ: 9.10 (1H, s), 8.48 (1H, d, J = 1.7 Hz), 8.15 (1H, dd, J = 8.5, 0.5 Hz), 7.88 (1H, dd, J = 8.6, 1.8 Hz), 7.28-7.24 (1H, m), 7.14 (1H, td, J = 7.4, 1.7 Hz), 7.04 (1H, t, J = 7.6 Hz), 6.63 (1H, s), 4.73 (2H, d, J = 5.6 Hz), 2.63 (2H, t, J = 7.5 Hz), 1.69-1.60 (2H, m), 0.96 (3H, t, J = 7.2 Hz). |
| 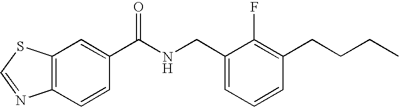 | 1H-NMR (CDCl3) δ: 9.10 (1H, s), 8.48 (1H, d, J = 1.7 Hz), 8.15 (1H, d, J = 8.5 Hz), 7.88 (1H, dd, J = 8.6, 1.8 Hz), 7.27-7.23 (1H, m), 7.14 (1H, td, J = 7.4, 1.8 Hz), 7.04 (1H, t, J = 7.6 Hz), 6.68 (1H, s), 4.72 (2H, d, J = 5.8 Hz), 2.65 (2H, t, J = 7.5 Hz), 1.63- 1.55 (2H, m), 1.37 (2H, td, J = 14.9, 7.4 Hz), 0.93 (3H, t, J = 7.4 Hz). |
| 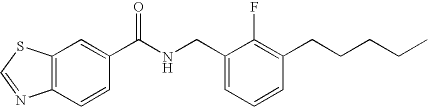 | 1H-NMR (CDCl3) δ: 9.11 (1H, s), 8.49 (1H, d, J = 1.4 Hz), 8.17 (1H, d, J = 8.5 Hz), 7.88 (1H, dd, J = 8.5, 1.7 Hz), 7.31-7.24 (1H, m), 7.15 (1H, td, J = 7.5, 1.4 Hz), 7.05 (1H, t, J = 7.5 Hz), 6.56 (1H, s), 4.73 (2H, d, J = 5.8 Hz), 2.65 (2H, t, J = 7.7 Hz), 1.65- 1.57 (2H, m), 1.36-1.32 (4H, m), 0.90 (3H, t, J = 6.9 Hz). |
| 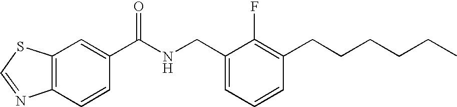 | 1H-NMR (CDCl3) δ: 9.11 (1H, s), 8.49 (1H, d, J = 1.7 Hz), 8.16 (1H, d, J = 8.5 Hz), 7.88 (1H, dd, J = 8.6, 1.6 Hz), 7.28-7.24 (1H, m), 7.15 (1H, t, J = 7.4 Hz), 7.05 (1H, t, J = 7.6 Hz), 6.57 (1H, s), 4.73 (2H, d, J = 5.8 Hz), 2.65 (2H, t, J = 7.6 Hz), 1.64-1.57 (2H, m), 1.37-1.29 (6H, m), 0.88 (3H, t, J = 6.9 Hz). |
| 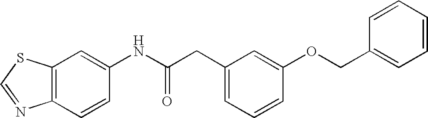 | 1H-NMR (DMSO-D6) δ: 3.66 (2H, s), 5.09 (2H, s), 6.89-6.95 (2H, m), 7.03 (1H, s), 7.25 (1H, t, J = 7.8 Hz), 7.30-7.33 (1H, m), 7.36-7.39 (2H, m), 7.44-7.46 (2H, m), 7.60 (1H, dd, J = 8.8, 2.0 Hz), 8.01 (1H, d, J = 8.8 Hz), 8.54 (1H, d, J = 2.0 Hz), 9.26 (1H, s), 10.43 (1H, s). |
| 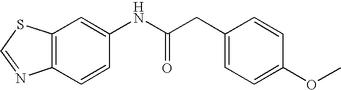 | 1H-NMR (CDCl3) δ: 8.90 (1H, s), 8.50 (1H, d, J = 1.9 Hz), 7.99 (1H, d, J = 8.9 Hz), 7.28 (2H, d, J = 8.7 Hz), 7.24 (1H, s), 7.18 (1H, dd, J = 8.7, 2.2 Hz), 6.96 (2H, d, J = 8.7 Hz), 3.84 (3H, s), 3.74 (2H, s). |
| 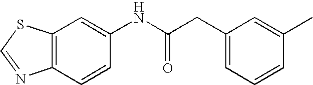 | 1H-NMR (CDCl3) δ: 8.90 (1H, s), 8.50 (1H, d, J = 1.9 Hz), 7.99 (1H, d, J = 8.7 Hz), 7.32 (1H, t, J = 7.7 Hz), 7.25 (1H, s), 7.19-7.15 (4H, m), 3.76 (2H, s), 2.39 (3H, s). |
| 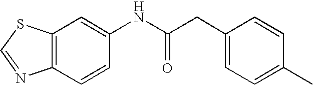 | 1H-NMR (CDCl3) δ: 8.90 (1H, s), 8.49 (1H, d, J = 1.9 Hz), 7.98 (1H, d, J = 8.9 Hz), 7.25-7.23 (5H, m), 7.17 (1H, dd, J = 8.8, 2.1 Hz), 3.76 (2H, s), 2.39 (3H, s). |
| 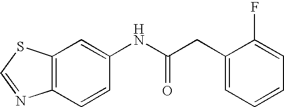 | 1H-NMR (CDCl3) δ: 8.91 (1H, s), 8.51 (1H, d, J = 1.9 Hz), 8.01 (1H, d, J = 8.7 Hz), 7.42-7.32 (3H, m), 7.26-7.24 (1H, m), 7.21-7.12 (2H, m), 3.80 (2H, s). |
| 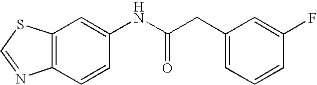 | 1H-NMR (CDCl3) δ: 8.91 (1H, s), 8.50 (1H, d, J = 1.7 Hz), 8.01 (1H, d, J = 8.7 Hz), 7.42-7.36 (1H, m), 7.29 (1H, s), 7.22 (1H, dd, J = 8.7, 1.9 Hz), 7.15 (1H, d, J = 7.5 Hz), 7.11-7.04 (2H, m), 3.78 (2H, s). |
| 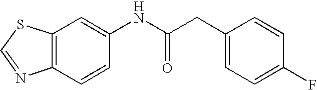 | 1H-NMR (CDCl3) δ: 4.63 (2H, s), 6.96-6.99 (2H, m), 7.04-7.08 (2H, m), 7.44 (1H, dd J = 8.8, 2.0 Hz), 8.09 (1H, d, J = 8.8 Hz), 8.43 (1H, s), 8.61 (1H, d, J = 2.2 Hz), 8.95 (1H, s). |
| 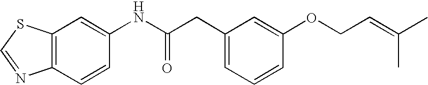 | 1H-NMR (CDCl3) δ: 8.90 (1H, s), 8.50 (1H, d, J = 1.9 Hz), 7.99 (1H, d, J = 8.9 Hz), 7.33 (1H, t, J = 7.6 Hz), 7.28 (1H, s), 7.18 (1H, dd, J = 8.8, 2.1 Hz), 6.94-6.90 (3H, m), 5.49 (1H, t, J = 6.8 Hz), 4.53 (2H, d, J = 6.8 Hz), 3.76 (2H, s), 1.79 (3H, s), 1.75 (3H, s). |
| 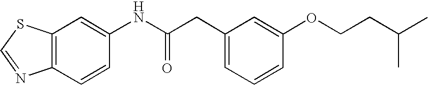 | 1H-NMR (CDCl3) δ: 8.90 (1H, s), 8.49 (1H, d, J = 1.9 Hz), 7.99 (1H, d, J = 8.9 Hz), 7.33 (1H, dd, J = 8.8, 7.6 Hz), 7.28 (1H, s), 7.18 (1H, dd, J = 8.8, 2.1 Hz), 6.93-6.89 (3H, m), 4.01 (2H, t, J = 6.8 Hz), 3.76 (2H, s), 1.88-1.79 (1H, m), 1.69 (2H, q, J = 6.8 Hz), 0.97 (6H, d, J = 6.5 Hz). |
| 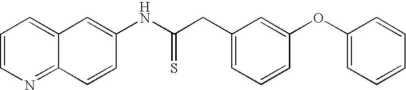 | 1H-NMR (CDCl3) δ: 4.30 (2H, s), 7.01-7.06 (4H, m), 7.12-7.17 (2H, m), 7.35-7.43 (4H, m), 7.59 (1H, dd, J = 9.0, 2.2 Hz), 8.08 (1H, d, J = 9.0 Hz), 8.15 (1H, d, J = 9.3 Hz), 8.52 (1H, d, J = 2.4 Hz), 8.65 (1H, s), 8.90 (1H, dd, J = 4.1, 1.7 Hz). |
| 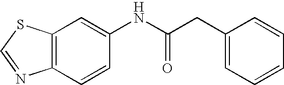 | 1H-NMR (DMSO-D6) δ: 3.69 (2H, s), 7.23-7.27 (1H, m), 7.31-7.37 (4H, m), 7.59-7.61 (1H, m), 8.01 (1H, d, J = 8.8 Hz), 8.54 (1H, d, J = 1.7 Hz), 9.25 (1H, s), 10.5 (1H, s). |
| 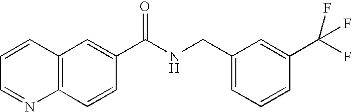 | 1H-NMR (CDCl3) δ: 4.77 (2H, d, J = 5.8 Hz), 6.81 (1H, br. s), 7.45-7.63 (5H, m), 8.07 (1H, dd, J = 7.2, 2.2 Hz), 8.15 (1H, d, J = 8.8 Hz), 8.21-8.23 (1H, m), 831-8.34 (1H, m), 8.98 (1H, dd, J = 4.4, 1.7 Hz). |
| 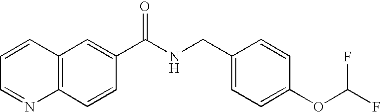 | 1H-NMR (CDCl3) δ: 4.70 (2H, d, J = 5.6 Hz), 6.51 (1H, t, J = 73.6 Hz), 6.59 (1H, br s), 7.13 (2H, d, J = 8.8 Hz), 7.40 (2H, d, J = 8.8 Hz), 7.47 (1H, dd, J = 8.3, 4.2 Hz), 8.06 (1H, dd, J = 8.8, 2.0 Hz), 8.16 (1H, d, J = 8.8 Hz), 8.24 (1H, d, J = 8.3 Hz), 8.33 (1H, d, J = 1.5 Hz), 8.88 (1H, dd, J = 4.2, 1.5 Hz). |
| 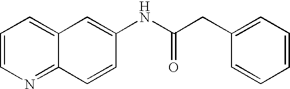 | 1H-NMR (CDCl3) δ: 3.80 (2H, s), 7.34-7.43 (7H, m), 7.63 (1H, br.s), 7.97 (1H, d, J = 9.0 Hz), 8.08 (1H, d, J = 8.3 Hz), 8.33 (1H, s), 8.79-8.80 (1H, m). |
| 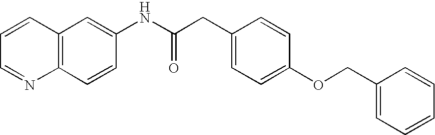 | 1H-NMR (CDCl3) δ: 3.75 (2H, s), 5.10 (2H, s), 7.03 (2H, d, J = 8.8 Hz), 7.26-7.46 (10H, m), 7.99 (1H, d, J = 9.0 Hz), 8.10 (1H, d, J = 8.3 Hz), 8.30 (1H, d, J = 2.2 Hz), 8.81 (1H, dd, J = 4.2, 1.7 Hz). |
| 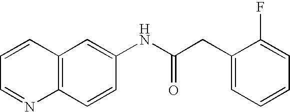 | 1H-NMR (CDCl3) δ: 3.82 (2H, s), 7.12-7.59 (7H, m), 8.01 (1H, d, J = 9.0 Hz), 8.09 (1H, d, J = 7.8 Hz), 8.34 (1H, s), 8.81 (1H, d, J = 2.4 Hz). |
| 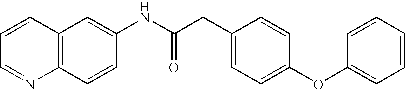 | 1H-NMR (CDCl3) δ: 3.78 (2H, s), 7.03-7.05 (4H, m), 7.12-7.16 (1H, m), 7.31-7.47 (7H, m), 8.01 (1H, d, J = 9.0 Hz), 8.10 (1H, d, J = 8.3 Hz), 8.34 (1H, d, J = 1.9 Hz), 8.82 (1H, dd, J = 4.2, 1.5 Hz). |
| 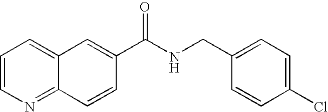 | 1H-NMR (CDCl3) δ: 4.69 (2H, d, J = 5.8 Hz), 6.58 (1H, br s), 7.34 (4H, s), 7.48-7.50 (1H, m), 8.06 (1H, dd, J = 8.8, 2.0 Hz), 8.16 (1H, d, J = 8.8 Hz), 8.23- 8.25 (1H, m), 8.33 (1H, d, J = 1.9 Hz), 8.99 (1H, dd, J = 4.2, 1.7 Hz). |
| 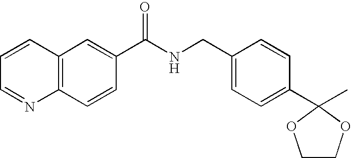 | 1H-NMR (CDCl3) δ: 1.66 (3H, s), 3.74-3.82 (2H, m), 4.00-4.09 (2H, m), 4.71 (2H, d, J = 5.6 Hz), 6.55 (1H, br s), 7.38 (2H, d, J = 8.1 Hz), 7.46-7.51 (3H, m), 8.06 (1H, dd, J = 8.8, 1.9 Hz), 8.16 (1H, d, J = 8.8 Hz), 8.24 (1H, d, J = 8.0 Hz), 8.34 (1H, d, J = 1.7 Hz), 8.99 (1H, dd, J = 4.2, 1.7 Hz). |
| 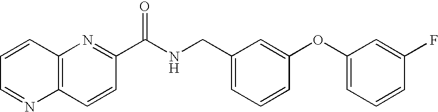 | 1H-NMR (CDCl3) δ: 4.74 (2H, d, J = 6.6 Hz), 6.70 (1H, dt, J = 10.2, 2.6 Hz), 6.76-6.81 (2H, m), 6.96 (1H, dd, J = 7.8, 2.7 Hz), 7.09 (1H, t, J = 2.0 Hz), 7.18-7.25 (2H, m), 7.36 (1H, t, J = 8.3 Hz), 7.71 (1H, dd, J = 8.9, 4.3 Hz), 8.40 (1H, dd, J = 8.8, 2.0 Hz), 8.53 (1H, br s), 8.57 (2H, s), 9.06 (1H, dd, J = 4.3, 1.8 Hz). |
| 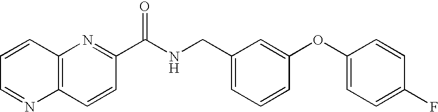 | 1H-NMR (CDCl3) δ: 4.72 (2H, d, J = 5.9 Hz), 6.86 (1H, dd, J = 8.5, 2.9 Hz), 6.95-7.04 (5H, m), 7.13 (1H, d, J = 6.6 Hz), 7.27-7.33 (1H, m), 7.40 (1H, dt, J = 31.0, 7.9 Hz), 8.39 (1H, dd, J = 8.4, 1.6 Hz), 8.55 (1H, br s), 8.57 (2H, s), 9.05 (1H, dd, J = 4.3, 1.6 Hz). |
| 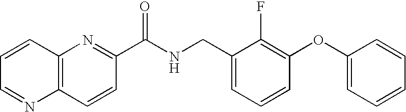 | 1H-NMR (CDCl3) δ: 4.84 (2H, d, J = 5.6 Hz), 6.97- 7.02 (3H, m), 7.06-7.13 (2H, m), 7.23-7.29 (1H, m), 7.31-7.36 (2H, m), 7.71 (1H, dd, J = 8.8, 4.1 Hz), 8.42 (1H, dd, J = 8.5, 1.7 Hz), 8.55-8.59 (3H, m), 9.06 (1H, dd, J = 4.1, 1.7 Hz). |
| 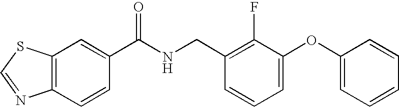 | 1H-NMR (CDCl3) δ: 4.78 (2H, d, J = 5.6 Hz), 6.75 (1H, br s), 6.96-7.01 (3H, m), 7.05-7.13 (2H, m), 7.20-7.24 (1H, m), 7.28-7.36 (2H, m), 7.89 (1H, dd, J = 8.5, 1.7 Hz), 8.16 (1H, d, J = 8.5 Hz), 8.49 (1H, d, J = 1.7 Hz), 9.11 (1H, s). |
| 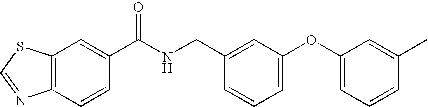 | 1H-NMR (CDCl3) δ: 2.32 (3H, s), 4.66 (2H, d, J = 5.6 Hz), 6.57 (1H, br s), 6.79-6.85 (2H, m), 6.91-7.16 (4H, m), 7.19-7.24 (1H, m), 7.28-7.33 (1H, m), 7.87 (1H, dd, J = 8.6, 1.6 Hz), 8.15 (1H, d, J = 8.5 Hz), 8.46-8.48 (1H, m), 9.10-9.12 (1H, m). |
| 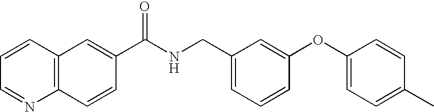 | 1H-NMR (CDCl3) δ: 2.33 (3H, s), 4.68 (2H, d, J = 5.8 Hz), 6.56 (1H, br s), 6.89-6.94 (3H, m), 7.01 (1H, s), 7.09-7.15 (3H, m), 7.28-7.33 (1H, m), 7.48 (1H, dd, J = 8.4, 4.2 Hz), 8.05 (1H, dd, J = 8.8, 2.1 Hz), 8.16 (1H, d, J = 8.8 Hz), 8.24 (1H, d, J = 8.4 Hz), 8.32 (1H, d, J = 1.8 Hz), 8.99 (1H, dd, J = 4.2, 1.8 Hz). |
| 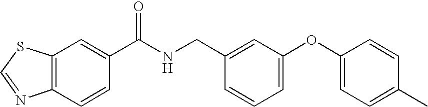 | 1H-NMR (CDCl3) δ: 2.33 (3H, s), 4.65 (2H, d, J = 5.6 Hz), 6.52 (1H, br s), 6.88-6.94 (3H, m), 6.98- 7.00 (1H, m), 7.06-7.16 (3H, m), 7.27-7.32 (1H, m), 7.86 (1H, dd, J = 8.5, 1.7 Hz), 8.16 (1H, d, J = 8.7 Hz), 8.47 (1H, d, J = 1.4 Hz), 9.11 (1H, s). |
| 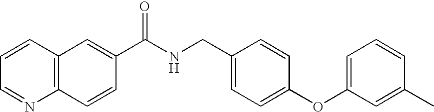 | 1H-NMR (CDCl3) δ: 2.33 (3H, s), 4.69 (2H, d, J = 5.8 Hz), 6.56 (1H, br s), 6.79-6.85 (2H, m), 6.93 (1H, d, J = 7.5 Hz), 6.98-7.03 (2H, m), 7.19-7.24 (1H, m), 7.36 (2H, d, J = 8.5 Hz), 7.48 (1H, dd, J = 8.3, 4.2 Hz), 8.07 (1H, dd, J = 8.7, 1.9 Hz), 8.16 (1H, d, J = 8.7 Hz), 8.22-8.26 (1H, m), 8.34 (1H, d, J = 1.7 Hz), 8.99 (1H, dd, J = 4.1, 1.7 Hz). |
| 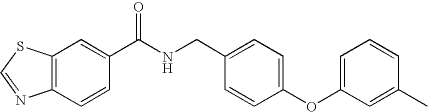 | 1H-NMR (CDCl3) δ: 2.33 (3H, s), 4.66 (2H, d, J = 5.6 Hz), 6.58 (1H, br s), 6.78-6.84 (2H, m), 6.93 (1H, dd, J = 7.5, 0.7 Hz), 6.99 (2H, d, J = 8.5 Hz), 7.19- 7.23 (1H, m), 7.33 (2H, d, J = 8.5 Hz), 7.89 (1H, dd, J = 8.3, 1.6 Hz), 8.15 (1H, d, J = 8.5 Hz), 8.50 (1H, d, J = 1.7 Hz), 9.10 (1H, s). |
| 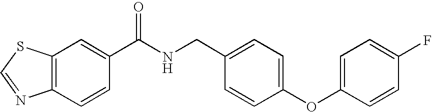 | 1H-NMR (CDCl3) δ: 4.66 (2H, d, J = 5.6 Hz), 6.50 (1H, br s), 6.94-7.06 (6H, m), 7.34 (2H, d, J = 8.7 Hz), 7.88 (1H, dd, J = 8.5, 1.7 Hz), 8.17 (1H, d, J = 8.5 Hz), 8.50 (1H, d, J = 1.7 Hz), 9.12 (1H, s). |
| 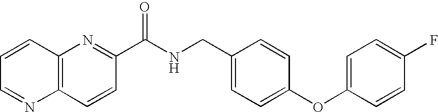 | 1H-NMR (CDCl3) δ: 4.72 (2H, d, J = 6.3 Hz), 6.95- 7.05 (6H, m), 7.38 (2H, d, J = 8.7 Hz), 7.70 (1H, dd, J = 8.6, 4.2 Hz), 8.37-8.41 (1H, m), 8.50 (1H, br s), 8.56 (1H, d, J = 8.7 Hz), 8.59 (1H, d, J = 8.7 Hz), 9.06 (1H, dd, J = 4.2, 1.4 Hz). |
| 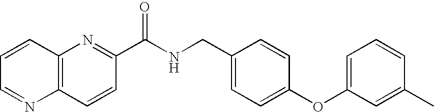 | 1H-NMR (CDCl3) δ: 2.33 (3H, s), 4.72 (2H, d, J = 6.0 Hz), 6.79-6.85 (2H, m), 6.90-6.94 (1H, m), 6.98- 7.03 (2H, m), 7.19-7.23 (1H, m), 7.38 (2H, d, J = 8.5 Hz), 7.69 (1H, dd, J = 8.7, 4.1 Hz), 8.38-8.41 (1H, m), 8.50 (1H, br s), 8.56 (1H, d, J = 8.7 Hz), 8.59 (1H, d, J = 8.7 Hz), 9.06 (1H, dd, J = 4.2, 1.6 Hz). |
| 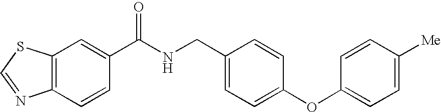 | 1H-NMR (CDCl3) δ: 2.34 (3H, s), 4.65 (2H, d, J = 5.6 Hz), 6.49 (1H, br s), 6.89-6.93 (2H, m), 6.95-6.99 (2H, m), 7.14 (2H, d, J = 8.5 Hz), 7.31-7.34 (2H, m), 7.88 (1H, dd, J = 8.5, 1.7 Hz), 8.16 (1H, d, J = 8.5 Hz), 8.50 (1H, d, J = 1.7 Hz), 9.11 (1H, s). |
| 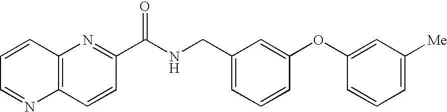 | 1H-NMR (CDCl3) δ: 2.31 (3H, s), 4.73 (2H, d, J = 6.0 Hz), 6.79-6.86 (2H, m), 6.89-6.95 (2H, m), 7.05- 7.08 (1H, m), 7.13-7.23 (2H, m), 7.30-7.35 (1H, m), 7.70 (1H, dd, J = 8.5, 4.1 Hz), 8.39 (1H, dd, J = 8.7, 1.7 Hz), 8.51 (1H, br s), 8.55-8.61 (2H, m), 9.06 (1H, dd, J = 4.1, 1.7 Hz). |
| 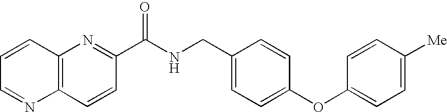 | 1H-NMR (CDCl3) δ: 2.33 (3H, s), 4.71 (2H, d, J = 6.0 Hz), 6.90-6.93 (2H, m), 6.96-7.00 (2H, m), 7.14 (2H, d, J = 8.0 Hz), 7.34-7.38 (2H, m), 7.69 (1H, dd, J = 8.7, 4.1 Hz), 8.37-8.41 (1H, m), 8.48 (1H, br s), 8.55-8.60 (2H, m), 9.05 (1H, dd, J = 4.1, 1.7 Hz). |
| 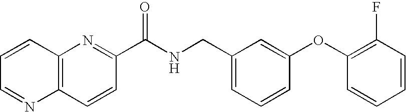 | 1H-NMR (CDCl3) δ: 4.73 (2H, d, J = 6.0 Hz), 6.88 (1H, dd, J = 8.2, 2.2 Hz), 7.05-7.21 (5H, m), 7.23- 7.35 (2H, m), 7.70 (1H, dd, J = 8.6, 4.2 Hz), 8.40 (1H, dd, J = 8.7, 1.4 Hz), 8.51 (1H, br s), 8.56-8.59 (2H, m), 9.06 (1H, dd, J = 4.1, 1.4 Hz). |
| 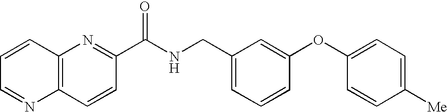 | 1H-NMR (CDCl3) δ: 2.32 (3H, s), 4.71 (2H, d, J = 6.0 Hz), 6.87-6.94 (3H, m), 7.03-7.05 (1H, m), 7.10- 7.14 (3H, m), 7.28-7.32 (1H, m), 7.70 (1H, dd, J = 8.6, 4.2 Hz), 8.39 (1H, dd, J = 8.6, 1.7 Hz), 8.50 (1H, br s), 8.56 (1H, d, J = 9.4 Hz), 8.58 (1H, d, J = 8.7 Hz), 9.06 (1H, dd, J = 4.1, 1.7 Hz). |

Next, formulation examples will be given below. It is to be noted that the term “part” indicates “part by weight.”

### Formulation Example 1

50 parts of the present compound (1), 3 parts of calcium lignosulfonate, 2 parts of magnesium lauryl sulfate, and 45 parts of synthetic hydrous silicon oxide were fully crushed and mixed, so as to obtain wettable powders. The same above operations were carried out with the exception that the present compound (1) was substituted with any one of the present compounds (2) to (231), so as to obtain various types of wettable powders.

### Formulation Example 2

20 parts of the present compound (1) and 1.5 parts of sorbitan trioleate were mixed into 28.5 parts of an aqueous solution that contained 2 parts of polyvinyl alcohol, and the mixed solution was then subjected to fine grinding according to a wet grinding method. Thereafter, 40 parts of an aqueous solution that contained 0.05 parts of xanthan gum and 0.1 part of aluminum magnesium silicate was added to the resultant, and 10 parts of propylene glycol was further added thereto. The obtained mixture was blended by stirring, so as to obtain a flowable formulation. The same above operations were carried out with the exception that the present compound (1) was substituted with any one of the present compounds (2) to (231), so as to obtain various types of flowable formulations.

### Formulation Example 3

2 parts of the present compound (1), 88 parts of kaoline clay, and 10 parts of talc were fully crushed and mixed, so as to obtain a powder product. The same above operations were carried out with the exception that the present compound (1) was substituted with any one of the present compounds (2) to (231), so as to obtain various types of powder products.

### Formulation Example 4

5 parts of the present compound (1), 14 parts of polyoxyethylene styryl phenyl ether, 6 parts of calcium dodecylbenzenesulfonate, and 75 parts of xylene were fully mixed, so as to obtain an emulsion. The same above operations were carried out with the exception that the present compound (1) was substituted with any one of the present compounds (2) to (231), so as to obtain various types of emulsions.

### Formulation Example 5

2 parts of the present compound (1), 1 part of synthetic hydrous silicon oxide, 2 parts of calcium lignosulfonate, 30 parts of bentonite, and 65 parts of kaoline clay were fully crushed and mixed. Thereafter, water was added to the mixture, and they were fully blended, followed by granulation and drying, so as to obtain a granule. The same above operations were carried out with the exception that the present compound (1) was substituted with any one of the present compounds (2) to (231), so as to obtain various types of granules.

### Formulation Example 6

10 parts of the present compound (1), 35 parts of white carbon that contained 50 parts of polyoxyethylene alkyl ether sulfate ammonium salts, and 55 parts of water were mixed, and the obtained mixture was then subjected to fine grinding according to a wet grinding method, so as to obtain a flowable formulation. The same above operations were carried out with the exception that the present compound (1) was substituted with any one of the present compounds (2) to (231), so as to obtain various types of flowable formulations.

Next, test examples will be given to demonstrate that the present invention is useful for the control of plant diseases.

### Test Example 1

### Test Regarding Effects of Preventing Infection by Cucumber Gray Mold (Botrytis cinerea)

A plastic pot was filled with sandy soil, and cucumber (variety: Sagami-Hanjiro) was then disseminated. The cucumber was allowed to grow in a green house for 12 days. The aforementioned flowable formulations comprising compounds 1, 2, 9, 12, 16, 18, 23, 24, 25, 26, 29, 30, 32, 36, 38, 39, 42, 43, 46, 47, 54, 60, 62, 64, 76, 77, 81, 90, 91, 95, 103, 105, 110, 116, 117, 118, 121, 123, 129, 132, 134, 135, 137, 145, 147, 148, 152, 155, 159, 185, 163, 164, 166, 171, 202, 204, 207, 208, 211, 219 were set at predetermine concentrations (500 ppm) by diluting with water, and they were then subjected to foliage application such that they could be sufficiently attached to the leaves of the aforementioned cucumber plants. After completion of the foliage application, the plants were air-dried. Thereafter, Botrytis cinerea spore-containing PDA medium was placed on the cotyledon surface. After such inoculation, the plants were left at 12° C. at a high humidity for 5 days, and the infected areas thereof were then examined. As a result, it was found that the infected areas of plants which had been treated with compounds 1, 2, 9, 12, 16, 18, 23, 24, 25, 26, 29, 30, 32, 36, 38, 39, 42, 43, 46, 47, 54, 60, 62, 64, 76, 77, 81, 90, 91, 95, 103, 105, 110, 116, 117, 118, 121, 123, 129, 132, 134, 135, 137, 145, 147, 148, 152, 155, 159, 185, 163, 164, 166, 171, 202, 204, 207, 208, 211, 219 were 10% or less of the infected areas of untreated plants.

### Test Example 2

### Test Regarding Effects of Preventing Infection by Cucumber Sclerotinia Rot (Sclerotinia sclerotiorum)

A plastic pot was filled with sandy soil, and cucumber (variety: Sagami-Hanjiro) was then disseminated. The cucumber was allowed to grow in a green house for 12 days. The aforementioned flowable formulations comprising compounds 1, 2, 3, 8, 9, 10, 11, 12, 13, 14, 15, 16, 17, 18, 21, 22, 23, 24, 25, 26, 27, 29, 30, 33, 34, 35, 36, 38, 39, 40, 41, 42, 43, 44, 45, 46, 47, 48, 49, 50, 51, 52, 53, 54, 55, 57, 59, 60, 62, 63, 64, 65, 66, 67, 68, 69, 70, 71, 72, 73, 74, 76, 77, 78, 79, 80, 81, 82, 83, 84, 85, 86, 87, 88, 89, 90, 91, 92, 93, 94, 95, 96, 97, 98, 99, 100, 102, 103, 104, 105, 106, 107, 108, 109, 110, 111, 112, 113, 114, 115, 116, 117, 118, 119, 120, 121, 123, 124, 125, 126, 127, 128, 129, 130, 131, 132, 133, 134, 135, 136, 137, 138, 139, 140, 141, 143, 144, 145, 146, 147, 148, 149, 151, 152, 153, 155, 156, 157, 159, 160, 158, 162, 163, 164, 165, 166, 167, 168, 169, 170, 171, 172, 173, 174, 175, 176, 177, 178, 179, 180, 181, 183, 185, 188, 190, 199, 200, 201, 202, 204, 205, 206, 207, 208, 209, 210, 211, 212, 213, 214, 215, 216, 217, 218, 219, 220, 221, 222, 223, 224, 225, 226, 227, 228, 229, 230, 231 were set at predetermine concentrations (500 ppm) by diluting with water, and they were then subjected to foliage application such that they could be sufficiently attached to the leaves of the aforementioned cucumber plants. After completion of the foliage application, the plants were air-dried. Thereafter, Sclerotinia sclerotiorum hypha-containing PDA medium was placed on the cucumber leaf surface. After such inoculation, the plants were left at 18° C. at a high humidity for 4 days, and the infected areas thereof were then examined. As a result, it was found that the infected areas of plants which had been treated with compounds 1, 2, 3, 8, 9, 10, 11, 12, 13, 14, 15, 16, 17, 18, 21, 22, 23, 24, 25, 26, 27, 29, 30, 33, 34, 35, 36, 38, 39, 40, 41, 42, 43, 44, 45, 46, 47, 48, 50, 52, 53, 54, 55, 57, 59, 60, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 93, 94, 95, 96, 97, 98, 99, 100, 102, 103, 104, 105, 106, 107, 108, 109, 110, 111, 112, 113, 115, 116, 117, 118, 119, 120, 121, 123, 124, 125, 127, 128, 129, 130, 131, 132, 133, 134, 135, 136, 137, 138, 139, 140, 141, 143, 144, 145, 146, 147, 148, 149, 150, 151, 152, 153, 155, 156, 157, 159, 160, 158, 162, 163, 164, 165, 166, 167, 168, 169, 170, 171, 172, 173, 174, 175, 176, 177, 178, 179, 180, 181, 183, 185, 190, 199, 200, 201, 202, 204, 205, 206, 207, 208, 209, 210, 211, 212, 213, 214, 215, 216, 217, 218, 219, 220, 221, 222, 223, 224, 225, 226, 227, 228, 229, 230, 231 were 10% or less of the infected areas of untreated plants. The infected areas of plants which had been treated with compounds 49, 51, 92, 114, 126 and 188 were 30% or less of the infected areas of untreated plants.

### Test Example 3

### Test Regarding Effects of Preventing Infection by Tomato Late Blight (Phytophthora infestans)

A plastic pot was filled with sandy soil, and tomato (variety: Patio) was then disseminated. The tomato was allowed to grow in a green house for 20 days. The aforementioned flowable formulations comprising compounds 176 and 177 were set at predetermine concentrations (500 ppm) by diluting with water, and they were then subjected to foliage application such that they could be sufficiently attached to the leaves of the aforementioned nursery tomato plants. The plants were subjected to air-drying to such an extent that the diluted solution on the leave could be dried. Thereafter, Phytophthora infestans spore-containing water suspension was inoculated into the nursery tomato plants by spraying it. After completion of the inoculation, the plants were first left at 23° C. at a high humidity for 1 day, and they were then allowed to grow in a green house for 4 days. Thereafter, the infected areas thereof were examined. As a result, it was found that the infected areas of plants which had been treated with compounds 176 and 177 were 10% or less of the infected areas of untreated plants.

### Test Example 4

### Test Regarding Effects of Preventing Infection by Wheat Fusarium Head Blight (Fusarium culmorum)

A plastic pot was filled with sandy soil, and wheat (variety: Shirogane) was then disseminated. The wheat was allowed to grow in a green house for 10 days. The aforementioned flowable formulation comprising compound (4) was set at a predetermine concentration (500 ppm) by diluting with water, and it was then subjected to foliage application such that it could be sufficiently attached to the leaves of the aforementioned wheat plants. After completion of the foliage application, the plants were subjected to air-drying. Thereafter, Fusarium culmorum spore-containing water suspension was inoculated into the wheat plants by spraying it. After completion of the inoculation, the plants were first left at 23° C. at a high humidity in a dark place for 4 days, and they were then left under illumination for 3 days. Thereafter, the infected areas thereof were examined. As a result, it was found that the infected areas of plants which had been treated with compound (4) were 30% or less of the infected areas of untreated plants.

### Test Example 5

### Test Regarding Effects of Preventing Infection by Rice Blast Disease (Magnaporthe grisea)

A plastic pot was filled with bed soil, and rice (variety: Nihonbare) was then disseminated. The rice plants were allowed to grow in a green house for 12 days. The aforementioned flowable formulations comprising compounds 56, 122, and 161 were set at predetermine concentrations (500 ppm) by diluting with water, and they were then subjected to foliage application such that they could be sufficiently attached to the leaves of the aforementioned rice plants. After completion of the foliage application, the plants were subjected to air-drying. Thereafter, pots containing leaves infected with rice blast disease were left at rest around the rice plants. All the rice plants were left at a high humidity only during the night. Five days after the inoculation, the infected areas thereof were examined. As a result, it was found that the infected areas of plants which had been treated with compounds 121 and 161 were 10% or less of the infected areas of untreated plants and the affected areas of plants which had been treated with compound 56 were 30% or less of the infected areas of untreated plants.

### Test Example 6

### Test Regarding Effects of Preventing Infection by Cucumber Sclerotical Disease by Soil Treatment (Irrigation) (Sclerotinia sclerotiorum)

A plastic pot was filled with sandy soil, and cucumber (variety: Sagami-Hanjiro) was then disseminated. The cucumber plants were allowed to grow in a green house for 12 days. The aforementioned flowable formulations comprising compounds 1, 33, 42, 48, 52, 60, 62, 65, 70, 74, 83, 84, 94, 105, 117, 118, 145, 159, 163, 170, 175, 176, 177, 185, and 207 were diluted with water, and 1 mg of such a compound was applied to the aforementioned pot by a soil irrigation treatment. After completion of the treatment, the plants were allowed to grow in a green house for 1 week. Thereafter, Sclerotinia sclerotiorum hypha-containing PDA medium was placed on the leaves of the cucumber plants. After such inoculation, the plants were left at 18° C. at a high humidity for 4 days, and the infected areas thereof were then examined. As a result, it was found that the infected areas of plants which had been treated with compounds 1, 33, 42, 48, 52, 60, 62, 65, 70, 74, 83, 84, 94, 105, 117, 118, 145, 159, 163, 170, 175, 176, 177, 185, and 207 were 10% or less of the infected areas of untreated plants.

### Test Example 7

### Test Regarding Effects of Treating Grape Downy Mildew (Plasmopara viticola)

A plastic pot was filled with sandy soil, and grape (variety: Berry-A) was then disseminated. The grape plants were allowed to grow in a green house for 40 days. A water suspension of zoosporangium of Plasmopara viticola was inoculated to the aforementioned pot by spaying it. Thereafter, the grape plants were left at 23° C. at a high humidity for 1 day, and they were then air-dried, so as to obtain grape downy mildew-infected nursery plants. The aforementioned flowable formulation comprising compound 189 was set at a predetermine concentration (500 ppm) by diluting with water, and it was then subjected to foliage application such that it could be sufficiently attached to the leaves of the aforementioned nursery grape plants. After completion of the foliage application, the plants were subjected to air-drying, and they were then left at 23° C. in a green house for 5 days. Thereafter, the plants were further left at 23° C. at a high humidity for 1 day. Thereafter, the infected areas thereof were examined. As a result, it was found that the infected areas of plants which had been treated with compound 189 were 10% or less of the infected areas of untreated plants.

### Test Example 8

### Test Regarding Effects of Preventing Infection by Tomato Wilt by Soil Treatment (Irrigation)

A plastic pot is filled with soil contaminated with tomato wilt (Fusarium oxysporum), and tomato (variety: Patio) is then disseminated. The aforementioned flowable formulations comprising compounds 1 to 231 are diluted with water, and 10 mg of such a compound is applied to the aforementioned pot by a soil irrigation treatment. The tomato plants are then allowed to grow in a green house for 1 month. Thereafter, the resultant plants are compared with untreated plants. As a result, it is found that no blotches are found in the plants treated with compounds 1 to 231, and that the treated plants favorably grow at a level equivalent to that of plants that grow have in noncontaminated soil.

### Test Example 9

A plastic pot is filled with soil contaminated with potato blue stem (Verticillium albo-atrum, V. dahliae, V. nigrescens), and potato (variety: Danshaku) is then planted. The aforementioned flowable formulations comprising compounds 1 to 231 are diluted with water, and 10 mg of such a compound is applied to the aforementioned pot by a soil irrigation treatment. The potato plants are then allowed to grow in a green house for 2 months. Thereafter, the resultant plants are compared with untreated plants. As a result, it is found that no blotches are found in the plants treated with compounds 1 to 231, and that the treated plants favorably grow at a level equivalent to that of plants that grow in noncontaminated soil.

### Test Example 10

The aforementioned flowable formulations comprising compounds 1 to 231 are applied to unhulled rice contaminated with rice bakanae disease (Gibberella fujikuroi) by a seed treatment at a degree of 200 g of each compound to 100 kg of seeds. Thereafter, the rice plants are then allowed to grow in a green house for 1 month. Thereafter, the resultant plants are compared with untreated plants. As a result, it is found that the plants treated with compounds 1 to 231 favorably grow at a level equivalent to that of plants that have grown from noncontaminated unhulled rice.

### Test Example 11

The aforementioned flowable formulations comprising compounds 1 to 231 are applied to malt seeds contaminated with malt damping off (Gaeumannomyces graminis) by a seed treatment at a degree of 200 g of each compound to 100 kg of seeds. Thereafter, the malt plants are then allowed to grow in a green house for 1 month. Thereafter, the resultant plants are compared with untreated plants. As a result, it is found that the plants treated with compounds 1 to 231 favorably grow at a level equivalent to that of plants that grow from noncontaminated seeds.

### INDUSTRIAL APPLICABILITY

According to the present invention, plant diseases caused by plant pathogenic microbes except for Aspergillus can be controlled or prevented.

1. An agricultural composition for controlling plant diseases caused by plant pathogenic microbes except for Aspergillus, comprising a compound represented by formula (I), a salt thereof, or a hydrate thereof: 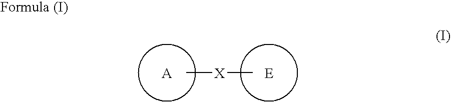
wherein A represents a 6-quinolyl group, a benzothiazol-6-yl group, or a [1.5]naphthylidin-2-yl group; X represents a group represented by formula —NH—C(═Y)—CH₂— or a group represented by formula —C(═Y)—NH—CH₂—; Y represents an oxygen atom, a sulfur atom, or NR^{Y} (wherein R^{Y} represents a C1-6 alkoxy group or a cyano group); and E represents a furyl group, a thienyl group, a pyrrolyl group, a tetrazolyl group, a thiazolyl group, a pyrazolyl group, or a phenyl group;
wherein A may optionally have one to three substituents selected from the following substituent group a-1 and substituent group a-2, and E may optionally have one or two substituents selected from the following substituent group a-1 and substituent group a-2:
[Substituent group a-1]
halogen atom, hydroxyl group, mercapto group, cyano group, carboxyl group, C1-6 alkyl group, C2-6 alkenyl group, C2-6 alkynyl group, C3-8 cycloalkyl group, C6-10 aryl group, 5- to 10-membered heterocyclic group, C3-8 cycloalkyl C1-6 alkyl group, C3-8 cycloalkylidene C1-6 alkyl group, C6-10 aryl C1-6 alkyl group, C6-10 aryl C2-6 alkenyl group, 5- to 10-membered heterocyclic C1-6 alkyl group, C1-6 alkoxy group, C2-6 alkenyloxy group, C2-6 alkynyloxy group, C3-8 cycloalkoxy group, C6-10 aryloxy group, C3-8 cycloalkyl C1-6 alkoxy group, C6-10 aryl C1-6 alkoxy group, 5- to 10-membered heterocyclic C1-6 alkoxy group, C1-6 alkylthio group, C2-6 alkenylthio group, C2-6 alkynylthio group, C3-8 cycloalkylthio group, C6-10 arylthio group, C3-8 cycloalkyl C1-6 alkylthio group, C6-10 aryl C1-6 alkylthio group, 5- to 10-membered heterocyclic C1-6 alkylthio group, mono-C1-6 alkylamino group, mono-C2-6 alkenylamino group, mono-C2-6 alkynylamino group, mono-C3-8 cycloalkylamino group, mono-C6-10 arylamino group, mono-C3-8 cycloalkyl C1-6 alkylamino group, mono-C6-10 aryl C1-6 alkylamino group, mono-5- to 10-membered heterocyclic C1-6 alkylamino group, di-C1-6 alkylamino group, N—C2-6 alkenyl-N—C1-6 alkylamino group, N—C2-6 alkynyl-N—C1-6 alkylamino group, N—C3-8 cycloalkyl-N—C1-6 alkylamino group, N—C6-10 aryl-N—C1-6 alkylamino group, N—C3-8 cycloalkyl C1-6 alkyl-N—C1-6 alkylamino group, N—C6-10 aryl C1-6 alkyl-N—C1-6 alkylamino group, N-5- to 10-membered heterocyclic C1-6 alkyl-N—C1-6 alkylamino group, C1-6 alkylcarbonyl group, C1-6 alkoxycarbonyl group, C1-6 alkylsulfonyl group, a group represented by formula —C(═N—R^{a1})R^{a2} (wherein R^{a1} represents a hydroxyl group or a C1-6 alkoxy group; and R^{a2} represents a hydrogen atom or a C1-6 alkyl group), C6-10 aryloxy C1-6 alkyl group, and 5- to 10-membered heterocyclic oxy C1-6 alkyl group; and
[Substituent group a-2]
C1-6 alkyl group, C2-6 alkenyl group, C2-6 alkynyl group, C3-8 cycloalkyl group, C6-10 aryl group, 5- to 10-membered heterocyclic group, C3-8 cycloalkyl C1-6 alkyl group, C6-10 aryl C1-6 alkyl group, 5- to 10-membered heterocyclic C1-6 alkyl group, C1-6 alkoxy group, C2-6 alkenyloxy group, C2-6 alkynyloxy group, C3-8 cycloalkoxy group, C6-10 aryloxy group, C3-8 cycloalkyl C1-6 alkoxy group, C6-10 aryl C1-6 alkoxy group, 5- to 10-membered heterocyclic C1-6 alkoxy group, C1-6 alkylthio group, C2-6 alkenylthio group, C2-6 alkynylthio group, C3-8 cycloalkylthio group, C6-10 arylthio group, C3-8 cycloalkyl C1-6 alkylthio group, C6-10 aryl C1-6 alkylthio group, 5- to 10-membered heterocyclic C1-6 alkylthio group, mono-C1-6 alkylamino group, mono-C2-6 alkenylamino group, mono-C2-6 alkynylamino group, mono-C3-8 cycloalkylamino group, mono-C6-10 arylamino group, mono-C3-8 cycloalkyl C1-6 alkylamino group, mono-C6-10 aryl C1-6 alkylamino group, mono-5- to 10-membered heterocyclic C1-6 alkylamino group, di-C1-6 alkylamino group, N—C2-6 alkenyl-N—C1-6 alkylamino group, N—C2-6 alkynyl-N—C1-6 alkylamino group, N—C3-8 cycloalkyl-N—C1-6 alkylamino group, N—C6-10 aryl-N—C1-6 alkylamino group, N—C3-8 cycloalkyl C1-6 alkyl-N—C1-6 alkylamino group, N—C6-10 aryl C1-6 alkyl-N—C1-6 alkylamino group, N-5- to 10-membered heterocyclic C1-6 alkyl-N—C1-6 alkylamino group, C6-10 aryloxy C1-6 alkyl group, and 5- to 10-membered heterocyclic oxy C1-6 alkyl group; wherein each group described in the substituent group a-2 has one to three substituents selected from the following substituent group b:
[Substituent group b]
halogen atom, hydroxyl group, mercapto group, cyano group, carboxyl group, amino group, carbamoyl group, nitro group, C1-6 alkyl group, C3-8 cycloalkyl group, C6-10 aryl group, 5- to 10-membered heterocyclic group, C1-6 alkoxy group, C6-10 aryloxy group, 5- to 10-membered heterocyclic oxy group, C1-6 alkoxycarbonyl group, C1-6 alkylsulfonyl group, trifluoromethyl group, trifluoromethoxy group, mono-C1-6 alkylamino group, di-C1-6 alkylamino group, mono-C6-10 arylamino group optionally having one amino group or one aminosulfonyl group, and N—C6-10 aryl C1-6 alkyl-N—C1-6 alkylamino group optionally having one amino group.

2. The agricultural composition according to claim 1, wherein, in formula (I), A represents a 6-quinolyl group, a [1.5]naphthylidin-2-yl group, or a benzothiazol-6-yl group (wherein A may optionally have one to three substituents selected from the following substituent group c-1 and substituent group c-2):
[Substituent group c-1]
halogen atom, C1-6 alkyl group, C2-6 alkenyl group, C2-6 alkynyl group, C3-8 cycloalkyl group, C6-10 aryl group, 5- to 10-membered heterocyclic group, C3-8 cycloalkyl C1-6 alkyl group, C6-10 aryl C1-6 alkyl group, C6-10 aryl C2-6 alkenyl group, 5- to 10-membered heterocyclic C1-6 alkyl group, C1-6 alkoxy group, C2-6 alkenyloxy group, C2-6 alkynyloxy group, C3-8 cycloalkyl C1-6 alkoxy group, C6-10 aryl C1-6 alkoxy group, 5- to 10-membered heterocyclic C1-6 alkoxy group, mono-C2-6 alkylamino group, mono-C3-8 alkenylamino group, mono-C6-10 alkynylamino group, mono-C3-8 cycloalkylamino group, mono-C1-6 arylamino group, mono-C6-10 cycloalkyl C1-6 alkylamino group, mono-C1-6 aryl C1-6 alkylamino group, mono-5- to 10-membered heterocyclic C1-6 alkylamino group, C2-6 alkylcarbonyl group, and a group represented by formula —C(═N—OH)R^{a2} (wherein R^{a2} has the same meanings as defined above); and
[Substituent group c-2]
C1-6 alkyl group, C2-6 alkenyl group, C2-6 alkynyl group, C3-8 cycloalkyl group, C6-10 aryl group, 5- to 10-membered heterocyclic group, C3-8 cycloalkyl C1-6 alkyl group, C6-10 aryl C1-6 alkyl group, 5- to 10-membered heterocyclic C1-6 alkyl group, C1-6 alkoxy group, C2-6 alkenyloxy group, C2-6 alkynyloxy group, C3-8 cycloalkyl C1-6 alkoxy group, C6-10 aryl C1-6 alkoxy group, 5- to 10-membered heterocyclic C1-6 alkoxy group, mono-C1-6 alkylamino group, mono-C2-6 alkenylamino group, mono-C2-6 alkynylamino group, mono-C3-8 cycloalkylamino group, mono-C6-10 arylamino group, mono-C3-8 cycloalkyl C1-6 alkylamino group, mono-C6-10 aryl C1-6 alkylamino group, and mono-5- to 10-membered heterocyclic C1-6 alkylamino group (wherein each group described in the substituent group c-2 has one to three substituents selected from the following substituent group d):
[Substituent group d]
halogen atom, hydroxyl group, carboxyl group, amino group, carbamoyl group, C1-6 alkoxy group, mono-C1-6 alkylamino group, di-C1-6 alkylamino group, mono-C6-10 arylamino group optionally having one amino group or one aminosulfonyl group, N—C6-10 aryl C1-6 alkyl-N—C1-6 alkylamino group optionally having one amino group, cyano group, C6-10 aryl group, 5- to 10-membered heterocyclic group, and C1-6 alkoxycarbonyl group.

3. The agricultural composition according to claim 1, wherein, in formula (I), A represents a 6-quinolyl group.

4. The agricultural composition according to claim 1, wherein, in formula (I), A represents a [1.5]naphthylidin-2-yl group.

5. The agricultural composition according to claim 1, wherein, in formula (I), A represents a benzothiazol-6-yl group.

6. The agricultural composition according to claim 1,
wherein, in formula (I), X represents a group represented by formula [—C(═O)—NH—CH₂—].

7. The agricultural composition according to claim 1,
wherein, in formula (I), E represents a furyl group, a thienyl group, a pyrrolyl group, or a phenyl group (wherein E may optionally have one or two substituents selected from the substituent group a-1 and the substituent group a-2).

8. The agricultural composition according to claim 1,
wherein, in formula (I), E represents a furyl group, a thienyl group, a pyrrolyl group, or a phenyl group (wherein E may optionally have one or two substituents selected from the following substituent group e-1 and substituent group e-2):
[Substituent group e-1]
halogen atom, C1-6 alkyl group, C2-6 alkenyl group, C2-6 alkynyl group, C6-10 aryl group, C3-8 cycloalkyl C1-6 alkyl group, C3-8 cycloalkylidene C1-6 alkyl group, C6-10 aryl C1-6 alkyl group, C6-10 aryl C2-6 alkenyl group, 5- to 10-membered heterocyclic C1-6 alkyl group, C1-6 alkoxy group, C2-6 alkenyloxy group, C2-6 alkynyloxy group, C6-10 aryloxy group, C3-8 cycloalkyl C1-6 alkoxy group, C6-10 aryl C1-6 alkoxy group, 5- to 10-membered heterocyclic C1-6 alkoxy group, C6-10 arylthio group, C6-10 aryl C1-6 alkylthio group, mono-C6-10 arylamino group, mono-C6-10 aryl C1-6 alkylamino group, N—C6-10 aryl-N—C1-6 alkylamino group, N—C6-10 aryl C1-6 alkyl-N—C1-6 alkylamino group, C6-10 aryloxy C1-6 alkyl group, and 5- to 10-membered heterocyclic oxy C1-6 alkyl group; and
[Substituent group e-2]
C1-6 alkyl group, C2-6 alkenyl group, C2-6 alkynyl group, C6-10 aryl group, C3-8 cycloalkyl C1-6 alkyl group, C6-10 aryl C1-6 alkyl group, 5- to 10-membered heterocyclic C1-6 alkyl group, C1-6 alkoxy group, C2-6 alkenyloxy group, C2-6 alkynyloxy group, C6-10 aryloxy group, C3-8 cycloalkyl C1-6 alkoxy group, C6-10 aryl C1-6 alkoxy group, 5- to 10-membered heterocyclic C1-6 alkoxy group, C6-10 arylthio group, C6-10 aryl C1-6 alkylthio group, mono-C6-10 arylamino group, mono-C6-10 aryl C1-6 alkylamino group, N—C6-10 aryl-N—C1-6 alkylamino group, N—C6-10 aryl C1-6 alkyl-N—C1-6 alkylamino group, C6-10 aryloxy C1-6 alkyl group, and 5- to 10-membered heterocyclic oxy C1-6 alkyl group;
wherein each group described in the substituent group e-2 has one to three substituents selected from the following substituent group f:
[Substituent group f]
halogen atom, hydroxyl group, cyano group, amino group, nitro group, C3-8 cycloalkyl group, C1-6 alkoxy group, C6-10 aryloxy group, 5- to 10-membered heterocyclic oxy group, C1-6 alkoxycarbonyl group, C1-6 alkylsulfonyl group, mono-C6-10 arylamino group, trifluoromethyl group, trifluoromethoxy group, and C1-6 alkyl group.

9. The agricultural composition according to claim 1,
wherein, in formula (I), E represents a furyl group, a thienyl group, a pyrrolyl group, or a phenyl group (wherein E may optionally have one substituent selected from the following substituent group g-1 and substituent group g-2):
[Substituent group g-1]
C3-8 cycloalkyl C1-6 alkyl group, phenyl C1-6 alkyl group, furyl C1-6 alkyl group, thienyl C1-6 alkyl group, benzofuryl C1-6 alkyl group, benzothienyl C1-6 alkyl group, C1-6 alkoxy group, phenoxy group, C3-8 cycloalkyl C1-6 alkoxy group, phenyl C1-6 alkoxy group, furyl C1-6 alkoxy group, thienyl C1-6 alkoxy group, pyridyl C1-6 alkoxy group, phenoxy C1-6 alkyl group, and pyridyloxy C1-6 alkyl group; and
[Substituent group g-2]
C3-8 cycloalkyl C1-6 alkyl group, phenyl C1-6 alkyl group, furyl C1-6 alkyl group, thienyl C1-6 alkyl group, benzofuryl C1-6 alkyl group, benzothienyl C1-6 alkyl group, C1-6 alkoxy group, phenoxy group, C3-8 cycloalkyl C1-6 alkoxy group, phenyl C1-6 alkoxy group, furyl C1-6 alkoxy group, thienyl C1-6 alkoxy group, pyridyl C1-6 alkoxy group, phenoxy C1-6 alkyl group, and pyridyloxy C1-6 alkyl group;
wherein each group described in the substituent group g-2 has one to three substituents selected from the following substituent group h:
[Substituent group h]
halogen atom, hydroxyl group, cyano group, and C1-6 alkyl group.

10. The agricultural composition according to claim 1, wherein, in formula (I), E represents a 2-furyl group, a 2-thienyl group, a 3-pyrrolyl group, or a phenyl group (wherein E may optionally have one substituent selected from the substituent group g-1 and the substituent group g-2).

11. The agricultural composition according to claim 1, wherein, in formula (I), X represents a group represented by formula [—C(═O)—NH—CH₂—], wherein A represents a 6-quinolyl group, a [1.5]naphthylidin-2-yl group, or a benzothiazol-6-yl group, and E represents a 2-furyl group, a 2-thienyl group, a 3-pyrrolyl group, or a phenyl group (wherein E may optionally have one substituent selected from the substituent group g-1 or the substituent group g-2).

12. The agricultural composition according to claim 1, wherein the plant pathogenic microbes are plant pathogenic filamentous fungi except for Aspergillus.

13. A method for controlling or preventing plant diseases caused by plant pathogenic microbes except for Aspergillus, which comprises applying an effective amount of the agricultural composition according to claim 1 to useful crops.

14. The method according to claim 13, wherein the composition is applied by foliage application, soil treatment, or seed disinfection.

15. Use of the compound represented by formula (I), a salt thereof, or a hydrate thereof, according to claim 1, in the manufacture of an agricultural composition.
